(19) United States
(12) Patent Application Publication
(10) Pub. No.: US 2013/0011411 A1
(43) Pub. Date: Jan. 10, 2013

(54) METHODS AND COMPOSITIONS FOR THE DIAGNOSIS, PROGNOSIS, AND TREATMENT OF CANCER

(76) Inventors: Richard G. Pestell, Philadelphia, PA (US); Kongming Wu, Wynnewood, PA (US); Xuanmao Jiao, Rockville, MD (US); Sanjay Katiyar, Vienna, VA (US); Vladimir M. Popov, Philadelphia, PA (US); Mathew Casimiro, Cherry Hill, NJ (US)

(21) Appl. No.: 13/520,759

(22) PCT Filed: Jan. 6, 2011
(86) PCT No.: PCT/US11/20423

§ 371 Date: Jul. 5, 2012

Related U.S. Application Data
(60) Provisional application No. 61/292,749, filed on Jan. 6, 2010.

(51) Int. Cl. A61K 31/706; A61P 35/00; A61K 48/00; A61K 38/02; A61K 31/7068; A61K 39/395

(52) U.S. Cl. 424/158.1; 514/42; 514/49; 514/44 R; 514/19.3

(57) ABSTRACT

Methods and compositions for diagnosing and treating cancer, such as breast cancer are provided. In particular, methods and compositions relating to nucleic acids encoding DACH1 and DACH1 proteins are provided.

### RELATED APPLICATIONS

This application claims priority to U.S. Provisional Application No. 61/292,749 filed Jan. 6, 2010, the contents of which is incorporated by reference in its entirety.

### STATEMENT REGARDING FEDERALLY SPONSORED R&D

This invention was made with government support under Grant Nos. R01CA70896, R01CA75503, R01CA86072, P30CA56036, and R01CA132115-02 awarded by National Institutes of Health (NIH). The government has certain rights in the invention.

### REFERENCE TO SEQUENCE LISTING

The present application is being filed along with a Sequence Listing in electronic format. The Sequence Listing is provided as a file entitled GTWN017SEQ.TXT, created Jan. 5, 2011 which is about 3 KB in size. The information in the electronic format of the Sequence Listing is incorporated herein by reference in its entirety.

### FIELD OF THE INVENTION

Methods and compositions for diagnosing and treating cancer, such as breast cancer, are provided. In particular, methods and compositions relating to nucleic acids encoding DACH1 and DACH1 proteins are provided.

### BACKGROUND OF THE INVENTION

Cancer is a significant health problem throughout the world. Although advances have been made in detection and therapy of cancer, no vaccine or other universally successful method for prevention and/or treatment is currently available. Current therapies, which are generally based on a combination of chemotherapy or surgery and radiation, continue to prove inadequate in many patients.

Breast cancer, for example, is a significant health problem for women in the United States and throughout the world. Although advances have been made in detection and treatment of the disease, breast cancer remains the second leading cause of cancer-related deaths in women, affecting more than 180,000 women in the United States each year. For women in North America, the life-time odds of getting breast cancer are now one in eight.

No vaccine or other universally successful method for the prevention or treatment of breast cancer is currently available. Management of the disease currently relies on a combination of early diagnosis (through routine breast screening procedures) and aggressive treatment, which may include one or more of a variety of treatments such as surgery, radiotherapy, chemotherapy and hormone therapy. The course of treatment for a particular breast cancer is often selected based on a variety of prognostic parameters, including an analysis of specific tumor markers. However, the use of established markers often leads to a result that is difficult to interpret, and the high mortality observed in breast cancer patients indicates that improvements are needed in the treatment, diagnosis and prevention of the disease.

### SUMMARY OF THE INVENTION

In spite of considerable research into therapies for cancers, certain cancers, such as breast cancer, remain difficult to diagnose and treat effectively. Accordingly, there is a need in the art for improved methods for detecting and treating such cancers.

Methods and compositions for diagnosing and treating cancer, such as breast cancer, are provided. In particular, methods and compositions relating to DACH1 proteins and nucleic acids encoding DACH1 are provided.

Some embodiments include methods for selecting a treatment for a subject with cancer comprising: determining an expression level of a nucleic acid encoding DACH1 or DACH1 protein in a sample obtained from a subject in need of treatment for cancer; and selecting a treatment for the subject based on the determined expression level. Some embodiments also include measuring an expression level of one or more nucleic acids encoding a gene selected from the group consisting of Sox2, Klf4, and Nanog or one or more proteins selected from the group consisting of SOX2, KLF4, and NANOG in a sample obtained from the subject.

Some embodiments also include comparing the expression level of the nucleic acid encoding DACH1 or DACH1 protein and the expression level of the one or more nucleic acids encoding a gene selected from the group consisting of Sox2, Klf4, and Nanog or the one or more proteins selected from the group consisting of SOX2, KLF4, and NANOG in the sample to an expression level of a nucleic acid encoding DACH1 or DACH1 protein and an expression level of the one or more nucleic acids encoding a gene selected from the group consisting of Sox2, Klf4, and Nanog or the one or more proteins selected from the group consisting of SOX2, KLF4, and NANOG in normal tissue or cancerous tissue with a known metastatic potential. In some embodiments, the comparing expression levels comprises a decreased expression level of a nucleic acid encoding DACH1 or DACH1 protein and an increased expression level of the nucleic acid encoding a gene selected from the group consisting of Sox2, Klf4, and Nanog or one or more proteins selected from the group consisting of SOX2, KLF4, and NANOG, and said selecting comprises selecting a treatment to increase the expression level of DACH1 in a tumor cell of the subject. In some embodiments, the decrease in expression level is statistically significant, and wherein the increase in expression level is statistically significant. In some embodiments, the decreased expression level comprises a decrease of at least about 50%. In some embodiments, the increased expression level comprises an increase of at least about 2-fold.

In some embodiments, the treatment is selected from the group consisting of administering a DNA-methyltransferase inhibitor to the subject, and administering to the subject an anti-IL8 therapy. In some embodiments, the treatment comprises administering a DNA-methyltransferase inhibitor to the subject. In some embodiments, the DNA-methyltransferase inhibitor comprises a compound selected from the group consisting of 5-azacytidine and 5-aza-2′-deoxycytidine. In some embodiments, the treatment comprises administering to the subject an anti-IL8 therapy. In some embodiments, the anti-IL8 therapy comprises an immunoneutralizing antibody. In some embodiments, the sample comprises ex vivo tissue. In some embodiments, the sample comprises cells of a solid tumor. In some embodiments, the sample comprises the phenotype estrogen receptor negative, progesterone receptor negative and ERB2 negative. In some embodiments, the normal tissue or cancerous tissue is tissue from the subject. In some embodiments, the normal tissue or cancerous tissue comprises ex vivo tissue. In some embodiments, determining the expression level a nucleic acid encoding DACH1 comprises measuring the level of DACH1 mRNA in a cell of said sample. In some embodiments, determining an expression level of DACH1 protein in said sample comprises an immunoblot analysis. In some embodiments, the subject is human. In some embodiments, the cancer comprises the phenotype estrogen receptor negative, progesterone receptor negative and ERB2 negative. In some embodiments, the cancer is selected from breast cancer, kidney cancer, lung cancer, brain cancer, endometrial cancer, ovarian cancer, pancreatic cancer, and prostate cancer.

Some embodiments include methods for treating cancer comprising administering an isolated nucleic acid encoding DACH1 or a DACH1 protein to a subject in need thereof.

In some embodiments, the cancer is a solid tumor. In some embodiments, a reduction in a size of the solid tumor is obtained. In some embodiments, a reduction in a size of the solid tumor by at least about 10% is obtained. In some embodiments, a reduction in a size of the solid tumor by at least about 80% is obtained. In some embodiments, a reduction in a proportion of CD24^{-/low} cells in the solid tumor is obtained. In some embodiments, a reduction in a proportion of CD24^{-/low} cells in the solid tumor by at least about 10% is obtained. In some embodiments, a reduction in a proportion of CD24^{-/low} cells in the solid tumor by at least about 50% is obtained. In some embodiments, the cancer is selected from breast cancer, kidney cancer, lung cancer, brain cancer, endometrial cancer, ovarian cancer, pancreatic cancer, and prostate cancer. In some embodiments, the cancer comprises the phenotype estrogen receptor negative, progesterone receptor negative and ERB2 negative. In some embodiments, a reduction in an expression level of one or more genes selected from the group consisting of Sox2, Nanog, and Klf4 is obtained. In some embodiments, a reduction is obtained in an expression level of one or more genes associated with a signaling pathway selected from the group consisting of hematopoietic cell lineage, cellular communication, blood vessel development, and multicellular organismal development. In some embodiments, a decrease is obtained in an expression level of one or more genes associated with an acute inflammation response and cytokine-cytokine receptor interactions. In some embodiments, the isolated nucleic acid further comprises a tissue-specific promoter. In some embodiments, the subject is a mammal. In some embodiments, the mammal is a human.

Some embodiments include methods for reducing a metastatic potential of a tumor comprising contacting the tumor with an isolated nucleic acid encoding DACH1 or DACH1 protein. In some embodiments, the proportion of invasive cells of the tumor is reduced by at least about 10%. In some embodiments, the proportion of invasive cells is reduced by at least about 90%. In some embodiments, the isolated nucleic acid further comprises a tissue-specific promoter. In some embodiments, the tissue-specific promoter comprises a mammary-specific promoter. In some embodiments, the mammary-specific promoter is selected from the group consisting of whey acidic protein promoter, β casein promoter, lactalbumin promoter and β-lactoglobulin promoter. In some embodiments, the cancer comprises the phenotype estrogen receptor negative, progesterone receptor negative and ERB2 negative. In some embodiments, the cancer is selected from breast cancer, kidney cancer, lung cancer, brain cancer, endometrial cancer, ovarian cancer, pancreatic cancer, and prostate cancer

Some embodiments include methods for evaluating a metastatic potential of a tumor in a subject comprising measuring an expression level of a nucleic acid encoding DACH1 or DACH 1 protein in a sample obtained from the subject. Some methods also include comparing the expression level of the nucleic acid encoding DACH1 or DACH1 protein in the sample to an expression level of a nucleic acid encoding DACH1 or DACH1 protein in normal tissue or in cancerous tissue with a known metastatic potential. In some embodiments, a decreased expression level of a nucleic acid encoding DACH1 or DACH1 protein is indicative of the metastatic potential of the tumor. Some methods also include measuring an expression level of one or more nucleic acids encoding a gene selected from the group consisting of Sox2, Klf4, and Nanog or one or more proteins selected from the group consisting of SOX2, KLF4, and NANOG in a sample obtained from the subject. Some embodiments also include comparing the expression level of the nucleic acid encoding DACH1 or DACH1 protein and the expression level of the one or more nucleic acids encoding a gene selected from the group consisting of Sox2, Klf4, and Nanog or the one or more proteins selected from the group consisting of SOX2, KLF4, and NANOG in the sample to an expression level of a nucleic acid encoding DACH1 or DACH1 protein and an expression level of one or more nucleic acids encoding a gene selected from the group consisting of Sox2, Klf4, and Nanog or one or more proteins selected from the group consisting of SOX2, KLF4, and NANOG in normal tissue or cancerous tissue with a known metastatic potential. In some embodiments, a decreased expression level of a nucleic acid encoding DACH1 or DACH1 protein and an increased expression level of the nucleic acid encoding one or more nucleic acids encoding a gene selected from the group consisting of Sox2, Klf4, and Nanog or the one or more proteins selected from the group consisting of SOX2, KLF4, and NANOG is indicative of the metastatic potential of the tumor. In some embodiments, the tumor comprises the phenotype estrogen receptor negative, progesterone receptor negative and ERB2 negative.

Some embodiments include methods for identifying an agent to treat a disorder related to decreased expression of DACH1 comprising: contacting a cell with a test compound; and determining a change in the level of DACH1 protein or the level of a nucleic acid encoding DACH1 in the cell, wherein an increase in the level of DACH1 protein or the level of a nucleic acid encoding DACH1 in the cell is indicative of a compound useful to treat a disorder related to decreased expression of DACH1. Some embodiments also include determining the change in the level of a protein selected from the group consisting of Sox2, Nanog, and Klf4, or the level of a nucleic acid encoding a protein selected from the group consisting of Sox2, Nanog, and Klf4. In some embodiments, a reduction in the level of expression of a protein selected from the group consisting of Sox2, Nanog, and Klf4, or the level of a nucleic acid encoding a protein selected from the group consisting of Sox2, Nanog, and Klf4 is indicative of a compound useful to treat a disorder related to decreased expression of DACH1. Some methods also include determining the proportion of CD44⁺/CD24^{-low} cells in a population of the cells. In some embodiments, a reduction in the proportion of CD44⁺/CD24^{-low} cells in the population is indicative of a compound useful to treat a disorder related to decreased expression of DACH1.

Some embodiments also include methods of inhibiting growth of a cell comprising contacting said cell with an isolated nucleic acid encoding DACH1 or a DACH1 protein. In some embodiments, the cell comprises a cancer stem cell. In some embodiments, the cell comprises the phenotype estrogen receptor negative, progesterone receptor negative and ERB2 negative. In some embodiments, the cell comprises a cell selected from a breast cancer cell, kidney cancer cell, lung cancer cell, brain cancer cell, endometrial cancer cell, ovarian cancer cell, pancreatic cancer cell, and prostate cancer cell. In some embodiments, a reduction in an expression level of one or more genes selected from the group consisting of Sox2, Nanog, and Klf4 is obtained. In some embodiments, a reduction is obtained in an expression level of one or more genes associated with a signaling pathway selected from the group consisting of hematopoietic cell lineage, cellular communication, blood vessel development, and multicellular organismal development. In some embodiments, a decrease is obtained in an expression level of one or more genes associated with an acute inflammation response and cytokine-cytokine receptor interactions. In some embodiments, the isolated nucleic acid further comprises a tissue-specific promoter. In some embodiments, the cell comprises a mammalian cell. In some embodiments, the cell comprises a human cell.

Some embodiments include methods of increasing proliferation of a stem cell comprising contacting said cell with an isolated nucleic acid encoding a portion of an antisense DACH1 gene. In some embodiments, the cell comprises a stem cell selected form the group consisting of neural stem cell, hematopoietic stem cell, muscle stem cell, and germ cell. In some embodiments, an increase in an expression level of one or more genes selected from the group consisting of Sox2, Nanog, and Klf4 is obtained. In some embodiments, an increase is obtained in an expression level of one or more genes associated with a signaling pathway selected from the group consisting of hematopoietic cell lineage, cellular communication, blood vessel development, and multicellular organismal development. In some embodiments, an increase is obtained in an expression level of one or more genes associated with an acute inflammation response and cytokine-cytokine receptor interactions. In some embodiments, the isolated nucleic acid further comprises a tissue-specific promoter. In some embodiments, the cell comprises a mammalian cell. In some embodiments, the cell comprises a human cell.

### BRIEF DESCRIPTION OF THE DRAWINGS

FIG. 1A shows a Western blot for DACH1 abundance of breast cancer cell lines, β-actin was used as a loading control (Cell lines include 1: MCF7; 2; SKBR3: 3: MDA-MB-453; 4: T47D; 5: MDA-MB-231; 6: Hs578T).

FIG. 1B shows a graph of normalized mRNA expression of DACH1 in breast cancer cell lines. (Cell lines include 1: MCF7; 2; SKBR3: 3: MDA-MB-453; 4: T47D; 5: MDA-MB-231; 6: Hs578T).

FIG. 1C shows a FACS display of CD44 and CD24 for various cells lines.

FIG. 1D Left and middle panels show photomicrographs of DACH1 expression in normal breast epithelium and triple negative invasive human breast cancer samples, respectively. FIG. 1D right panel shows a graph of relative DACH1 expression in normal breast epithelium and triple negative invasive human breast cancer samples.

FIG. 2A shows a photograph of a nude mouse injected with equal numbers of Met-1 cells co-transduced with either control vector or DACH1 expression plasmid.

FIG. 2B shows a graph of tumor volume of Met-1 cells implanted in nude mice over time. Analysis was conducted of N=5, data are mean±SEM.

FIG. 2C shows a graph of tumor weight of Met-1 cells co-transduced with either control vector or DACH1 expression plasmid after 35 days implantation. N=5 separate mice.

FIG. 2D shows a photograph of tumors of Met-1 cells co-transduced with either control vector or DACH1 expression plasmid after 35 days implantation.

FIG. 3 shows a graph of tumor formation rate in Met-1 cells transduced with vector or expression plasmid in a serial transplantation study.

FIG. 4A shows FACS displays of CD24/CD29 double staining of cells isolated from Met-1-GFP or Met-1-DACH1 tumors (Data are mean±SEM, N=5, P<0.001).

FIG. 4B shows a graph of percentage of CD24 cells.

FIG. 5 Left panel shows a FACS display for aldefluor staining of Met-1 cells transduced with either DACH1 or a mutant DACH1 defective in DNA binding (DACH ΔDS). FIG. 5 Right panel shows a graph of relative aldefluor staining in cells transduced with either DACH1 or DACH ΔDS.

FIG. 6A shows graphs of mean diameter of mamospheres in mammosphere assays of Met-1 cells transduced with a retroviral expression vector encoding DACH1 or control vector.

FIG. 6B shows a graph of relative mRNA expression of Sox2, Nanog, Klf-4, Oct4, and Myc in Met-1 cells transfected with vector encoding DACH1 or control vector (Data are mean±SEM, N=3).

FIG. 6C shows diagrams depicting the structures of DACH1 wild type or ΔDS mutant.

FIG. 6D shows graphs of relative activity for luciferase reporter gene assays of Sox-2 and Nanog promoters in cells transfected with DACH1 or ΔDS mutant constructs. The data are mean±SEM of N>5 separate transfections (P<0.001).

FIG. 7A shows FACS displays for CD24/CD44 double staining of Met-1 cells in vitro expressing DACH1 or control vector.

FIG. 7B shows a graph of the percentage population comprising CD24⁻/CD44⁺ cells in cells transfected with DACH1 or vector only.

FIG. 7C shows a schematic representation of methods for analysis of a Met-1 population.

FIG. 7D shows FACS displays for CD24^{low}/CD44^{high} and CD24^{high}/CD44^{high}.

FIG. 7E shows a graph of mammosphere formation for CD24^{low} and CD24^{high} cells.

FIG. 7F shows a graph of tumor volume in nude mice for CD24^{low} and CD24^{high} cells.

FIG. 8A and FIG. 8B depict a collagen gel invasion assay of control and DACH1 transduced Met-1 cells, respectively.

FIG. 8C is a graph of cell numbers/field for CD24^{low} and CD24^{high} Met-1 cells in a transwell migration assay.

FIG. 8D shows FACS displays for Met-1 cells co-cultured with conditioned medium from GFP or DACH1 expressing Met-1 cells.

FIG. 8E shows a graph of percentage of CD24^{low} Met-1 cells expressing either GFP or DACH1 co-cultured with conditioned medium from either GFP or DACH1 expressing Met-1 cells for 48 hours.

FIG. 9A (Left panel) shows photomicrographs of Met-1 cells transfected with lentivirus shRNA vector to Dach1 (Met-1shDACH1) or control (Met-1shCTL) under phase contrast or fluorescence fields of view. FIG. 9A (Right panel) shows a Western blot of DACH1 expression in Met-1shDACH1 or Met-1shCTL.

FIG. 9B (Left panel) shows a FACS display CD44 and CD24 expression for Met-1shDACH1 or Met-1shCTL. FIG. 9B (Right panel) shows relative CD24/CD44 expression in Met-1shDACH1 or Met-1shCTL.

FIG. 9C shows graphs for mean diameter of mammospheres, mammosphere volume, and percentage mammosphere in Met-1shDACH1 or Met-1shCTL populations of cells. Data are mean±SEM of 5 separate experiments.

FIG. 9D depicts a FACS analysis of population of cells including MCF104A, MCF104A-Myc, and MCF104A-Myc-DACH1, and a Western blot analysis.

FIG. 10A shows a treeview display of microarray analysis of Met-1 cells in culture. DACH1 stable cell lines (N=3) vs. vector control (N=3).

FIG. 10B shows a diagram of a pathway analysis of microarray data from Met-1 cells expressing DACH1 or control vector using DAVID and Gene Ontology and KEGG data sets (N=6). Pathways are represented by enrichment score. NOS (Nanog, Oct, Sox) target pathways are repressed by DACH1 expression. The Myc target pathway was not affected by DACH1 (data not shown).

FIG. 10C shows a diagram of a gene set enrichment analysis from micro array analysis data of Met-1 cells using gene targets enriched in ES cells, such as Sox-2, Oct-4 or NOS.

FIG. 11A shows a graph of CD24low/CD44high staining of multiplicate transductions.

FIG. 11B depicts FACS analysis of Met-1 cells transduced with viral vectors encoding KLF4/c-Myc or Oct4/Sox2.

FIG. 12A depicts DACH1-dependent tag density at selected gene promoters. Arrow indicates the start site and direction of transcription.

FIG. 12B depicts chromatin immunoprecipitation assays of the Sox2 gene.

FIG. 12C depicts chromatin immunoprecipitation assays of the Nanog gene.

FIG. 13 depicts expression levels of DACH in distinct genetic subtypes of human breast cancer. Wisker plots indicate significant difference in abundance of DACH1 mRNA in the basal genotype of breast cancer.

FIG. 14 shows a table of results of a molecular pathway analysis was conducted with DAVID using Gene Ontology and KEGG pathway sets

### DETAILED DESCRIPTION OF THE PREFERRED EMBODIMENTS

The following description and examples illustrate a preferred embodiment of the present invention in detail. Those of skill in the art will recognize that there are numerous variations and modifications of this invention that are encompassed by its scope. Accordingly, the description of a preferred embodiment should not be deemed to limit the scope of the present invention.

The preferred embodiments relates to methods and compositions for diagnosing and treating cancer, such as breast cancer. In particular, methods relating to nucleic acids encoding DACH1 and DACH1 proteins are provided.

Self-renewing stem-like cells known as tumor initiating cells (TICs) have been described in the hematopoietic system, the breast, colon, brain, prostate tumors (see e.g., Al-Hajj M. Cancer stem cells and oncology therapeutics. Curr Opin Oncol 2007; 19: 61-4). Mouse mammary stem cells express specific cell surface markers and show self-renewing properties (Visvader J E, Lindeman G J. Mammary stem cells and mammopoiesis. Cancer Res 2006; 66: 9798-801). Only a small number of primary breast cancer cells form secondary tumors. These TICs form mammospheres similar to normal mammary gland stem cells (Dontu G, et al. In vitro propagation and transcriptional profiling of human mammary stem/progenitor cells. Genes Dev 2003; 17: 1253-70). When cultured under specific conditions, TICs or cancer stem cells can be enriched by fluorescence activated cell sorting for CD44⁺/CD24^{-low} cells (Al-Hajj M, et al. Prospective identification of tumorigenic breast cancer cells. PNAS USA 2003; 100: 3983-8).

The molecular circuitry controlling embryonic stem cells may also be active in certain tumors. Several key regulators of embryonic stem cell identity, such as Oct4 and Sox2, Eklf, and Nanog, are expressed in a subset of specific tumors (Boyer L A, et al. Core transcriptional regulatory circuitry in human embryonic stem cells. Cell 2005; 122: 947-56). Genes known to regulate features of mammary stem cells include expression of twist (Mani S A, Guo W, Liao M J, et al. The epithelial-mesenchymal transition generates cells with properties of stem cells. Cell 2008; 133: 704-15). Although, self-renewal of primitive hematopoietic stem cells requires p21^{CIP1} (Cheng T, et al. Hematopoietic stem cell quiescence maintained by p21cip1/waf1. Science 2000; 287: 1804-8), the role of tumor suppressors in regulating TICs particularly in breast cancer are poorly understood.

Some studies have demonstrated a correlation between poor prognosis breast cancer and reduced expression of the cell-fate determination factor DACH1 (Wu K, et al. DACH1 is a cell fate determination factor that inhibits Cyclin D1 and breast tumor growth. Mol Cell Biol 2006; 26: 7116-29). Several lines of evidence suggest Dachshund may function as a tumor suppressor. Initially cloned as a dominant inhibitor of Ellipse in Drosophila, the mammalian DACH1 gene inhibits breast cancer cellular DNA synthesis and proliferation in cultured cells. DACH1 inhibits contact-independent growth induced by c-jun in part by forming a physical interaction with c-Jun in the context of local chromatin at target AP-1 sites. The drosophila dac gene is a key member of the retinal determination gene network (RDGN) that specifies eye tissue identity. In Drosophila, a coordinated system of genes, including dachshund (dac), eyes absent (eya), ey, twin of eyeless (toy), teashirt (tsh) and sin oculis (so), Dac is expressed in progenitor cells and neurons of the mushroom body, a brain structure present in most arthropods, and Dac expression can induce ectopic eye formation in Drosophila (Silver S J, Rebay 1. Signaling circuitries in development: insights from the retinal determination gene network. Development 2005; 132: 3-13). The mammalian homologue of so is known as Six and altered expression of the Six family and DACH1 occurs in a variety of human tumors.

The cell-fate determination factor Dachshund was cloned as a dominant inhibitor of the hyperactive epidermal growth factor (EGFR) ellipse. The expression of Dachshund is lost in human breast cancer associated with poor prognosis. Breast tumor initiating cells (TIC) may contribute to tumor progression and therapy resistance. Described herein re-expression of Dachshund blocked breast tumor cell growth in vivo. TIC form non adherent mammospheres and can be enriched by cell sorting for CD44^{high}/CD24^{low} cells. DACH1 expression reduced mammosphere formation and the proportion of CD44^{high}/CD24^{low} breast tumor cells. Genome wide expression studies of mammary tumors expressing DACH1 demonstrated DACH1 repressed a molecular signature associated with stem cells. Mechanistic studies demonstrated DACH1 expression in breast tumors and DACH1 directly repressed the Nanog and Sox2 promoters via a conserved domain. The cell-fate determination factor Dachshund inhibits breast tumor growth and breast tumor initiating cells.

Methods provided herein include treating cancer by administering a DACH1 protein or an isolated nucleic acid encoding a DACH1 protein to a subject, e.g., a mammal, a human, in need thereof. The cancer can include a solid tumor, e.g., breast cancer. More methods include reducing the metastatic potential of a tumor. Such methods include contacting the tumor with a DACH1 protein or an isolated nucleic acid encoding a DACH1 protein. More methods include evaluating the metastatic potential of a tumor in a subject. Such methods can include measuring the expression level of a nucleic acid encoding DACH1 or the level of the DACH 1 protein in a sample obtained from the subject. More methods for evaluating the metastatic potential of a tumor can also include measuring the expression level of one or more nucleic acids encoding a gene that include Sox2, Klf4, and Nanog or one or more proteins that include SOX2, KLF4, and NANOG in a sample obtained from the subject.

### Methods of Diagnosis

Some embodiments relate to methods for diagnosis and prognosis of particular types of cancer, such as breast cancer. In some embodiments, the metastatic potential of a cancer is assessed by measuring the amount of a nucleic acid encoding DACH1 or the amount of DACH 1 protein in a biological sample. In some aspects of such embodiments, the amount of nucleic acid encoding DACH1 or DACH1 protein in a biological sample is compared to that of a control sample indicative of non-cancerous tissues, a particular stage of cancer, or cancer with metastatic potential.

In some embodiments, the level of a nucleic acid encoding a marker in addition to a nucleic acid encoding DACH1 or the level of a protein marker in addition to DACH1 is measured. For example, the additional marker may be Sox2, Klf4, or Nanog. A biological sample can be any sample suitable for measuring the level of a nucleic acid encoding DACH1, or for measuring DACH1 protein. For example, the biological sample can include blood, sera, sputum urine and tumor biopsies, including epithelial cells and breast cancer cells obtained from a patient.

Expression levels can be measured by various methods, such as levels of mRNA, levels of protein, and levels of biological activity of a protein or mRNA. Typically, the increase or decrease in expression of a marker is relative to a non-cancerous control.

Polynucleotide primers and probes may be used to detect the level of mRNA encoding DACH1 or an additional marker mRNA or protein, which is also indicative of the presence or absence of a cancer. In general, a marker sequence may be present at a level that is increased or decreased at least two-fold, preferably three-fold, and more in tumor tissue than in normal tissue of the same type from which the tumor arose. Expression levels of a particular marker sequence in tissue types different from that in which the tumor arose are irrelevant in certain diagnostic embodiments since the presence of tumor cells can be confirmed by observation of predetermined differential expression levels, e.g., about 2-fold, 5-fold, etc, in tumor tissue to expression levels in normal tissue of the same type.

In some embodiments, a decrease in the level of expression of a marker or nucleic acid encoding a marker, such as DACH1, in a sample relative to expression levels in normal tissue, or cancer with metastatic potential, can indicate the metastatic potential of a cancer. In such embodiments, the decrease can be about 2-fold, 5-fold, 10-fold, 100-fold, or more. In some embodiments, an increase in the level of expression in a sample of a marker such as Sox2, Klf4, or Nanog, relative to expression levels in normal tissue, or cancer with metastatic potential, can indicate the stage or metastatic potential of a cancer. In such embodiments, the increase can be about 2-fold, 5-fold, 10-fold, 100-fold, or more.

In certain embodiments, the presence, or metastatic potential of cancer can be assessed by comparing the level of expression of at least one marker in a biological sample and non-cancerous control sample. Such embodiments include measuring the level of expression of DACH1 protein or nucleic acid encoding DACH1. More embodiments can include measuring the level of expression of DACH1 and the level of expression of an additional marker.

Differential expression patterns can be utilized advantageously for diagnostic purposes. For example, in one aspect described herein, altered expression levels of DACH1 and, in some embodiments, an additional marker in tumor tissue relative to normal tissue of the same type, but not in other normal tissue types can be exploited diagnostically. For example, the presence of metastatic tumor cells, such as in a sample taken from the circulation or some other tissue site different from that in which the tumor arose, can be identified and/or confirmed by detecting altered expression of DACH1, and in some embodiments, an additional marker in the sample, for example using RT-PCR analysis or other methodologies for measuring nucleic acid levels. In many instances, it will be desired to enrich for tumor cells in the sample of interest using cell capture or other like techniques.

There are a variety of assay formats known to those of ordinary skill in the art for using a binding agent to detect polypeptide markers in a sample. See, e.g., Harlow and Lane, Antibodies: A Laboratory Manual, Cold Spring Harbor Laboratory, 1988. In general, the presence or absence of a cancer in a subject may be determined by (a) contacting a biological sample obtained from a subject with a binding agent; (b) detecting in the sample a level of polypeptide that binds to the binding agent; and (c) comparing the level of polypeptide with a predetermined cut-off value.

In a preferred embodiment, the assay involves the use of binding agent immobilized on a solid support to bind to and remove the polypeptide from the remainder of the sample. The bound polypeptide may then be detected using a detection reagent that contains a reporter group and specifically binds to the binding agent/polypeptide complex. Such detection reagents may comprise, for example, a binding agent that specifically binds to the polypeptide or an antibody or other agent that specifically binds to the binding agent, such as an anti-immunoglobulin, protein G, protein A or a lectin. In such embodiments, the binding agent can comprise an antibody or fragment thereof specific to DACH1. Alternatively, a competitive assay may be utilized, in which a polypeptide is labeled with a reporter group and allowed to bind to the immobilized binding agent after incubation of the binding agent with the sample. The extent to which components of the sample inhibit the binding of the labeled polypeptide to the binding agent is indicative of the reactivity of the sample with the immobilized binding agent. Suitable polypeptides for use within such assays include full length breast tumor proteins and polypeptide portions thereof to which the binding agent binds, for example the DACH1 protein or additional markers described herein.

The solid support may be any material known to those of ordinary skill in the art to which the binding agent may be attached. For example, the solid support may be a test well in a microtiter plate or a nitrocellulose or other suitable membrane. Alternatively, the support may be a bead or disc, such as glass, fiberglass, latex or a plastic material such as polystyrene or polyvinylchloride. The support may also be a magnetic particle or a fiber optic sensor, such as those disclosed, for example, in U.S. Pat. No. 5,359,681. The binding agent may be immobilized on the solid support using a variety of techniques known to those of skill in the art, which are amply described in the patent and scientific literature. The term “immobilization” as used herein is a broad term, and is to be given its ordinary and customary meaning to a person of ordinary skill in the art (and is not to be limited to a special or customized meaning), and refers without limitation to both noncovalent association, such as adsorption, and covalent attachment (which may be a direct linkage between the agent and functional groups on the support or may be a linkage by way of a cross-linking agent). Immobilization by adsorption to a well in a microtiter plate or to a membrane is preferred. In such cases, adsorption may be achieved by contacting the binding agent, in a suitable buffer, with the solid support for a suitable amount of time. The contact time varies with temperature, but is typically from about 1 hour to about 1 day. In general, contacting a well of a plastic microtiter plate (such as polystyrene or polyvinylchloride) with an amount of binding agent of from about 10 ng to about 10 μg, and preferably from about 100 ng to about 1 μg, is sufficient to immobilize an adequate amount of binding agent.

Covalent attachment of binding agent to a solid support may generally be achieved by first reacting the support with a bifunctional reagent that will react with both the support and a functional group, such as a hydroxyl or amino group, on the binding agent. For example, the binding agent may be covalently attached to supports having an appropriate polymer coating using benzoquinone or by condensation of an aldehyde group on the support with an amine and an active hydrogen on the binding partner (see, e.g., Pierce Immunotechnology Catalog and Handbook, 1991, at A12-A13).

In certain embodiments, the assay is a two-antibody sandwich assay. This assay may be performed by first contacting an antibody that has been immobilized on a solid support, commonly the well of a microtiter plate, with the sample, such that polypeptides within the sample are allowed to bind to the immobilized antibody. Unbound sample is then removed from the immobilized polypeptide-antibody complexes and a detection reagent (preferably a second antibody capable of binding to a different site on the polypeptide) containing a reporter group is added. The amount of detection reagent that remains bound to the solid support is then determined using a method appropriate for the specific reporter group.

More specifically, once the antibody is immobilized on the support as described above, the remaining protein binding sites on the support are typically blocked. Any suitable blocking agent known to those of ordinary skill in the art may be used, such as bovine serum albumin or TWEEN® 20 (a PEG(20) sorbitan monolaurate available from Sigma Chemical Co., St. Louis, Mo.). The immobilized antibody is then incubated with the sample, and polypeptide is allowed to bind to the antibody. The sample may be diluted with a suitable diluent, such as phosphate-buffered saline (PBS) prior to incubation. In general, an appropriate contact time (e.g., incubation time) is a period of time that is sufficient to detect the presence of polypeptide within a sample obtained from an individual with breast cancer. Preferably, the contact time is sufficient to achieve a level of binding that is at least about 95% of that achieved at equilibrium between bound and unbound polypeptide. Those of ordinary skill in the art will recognize that the time necessary to achieve equilibrium may be readily determined by assaying the level of binding that occurs over a period of time. At room temperature, an incubation time of about 30 minutes is generally sufficient.

Unbound sample may then be removed by washing the solid support with an appropriate buffer, such as PBS containing 0.1% TWEEN® 20. The second antibody, which contains a reporter group, may then be added to the solid support. Reporter groups are well known in the art.

The detection reagent is then incubated with the immobilized antibody-polypeptide complex for an amount of time sufficient to detect the bound detection reagent. An appropriate amount of time may generally be determined by assaying the level of binding that occurs over a period of time. Unbound detection reagent is then removed and bound detection reagent is detected using the reporter group. The method employed for detecting the reporter group depends upon the nature of the reporter group. For radioactive groups, scintillation counting or autoradiographic methods are generally appropriate. Spectroscopic methods may be used to detect dyes, luminescent groups and fluorescent groups. Biotin may be detected using avidin, coupled to a different reporter group (commonly a radioactive or fluorescent group or an enzyme). Enzyme reporter groups may generally be detected by the addition of substrate (generally for a specific period of time), followed by spectroscopic or other analysis of the reaction products.

To determine the presence or absence of a cancer, such as breast cancer, the signal detected from the reporter group that remains bound to the solid support is generally compared to a signal that corresponds to a predetermined cut-off value. In one embodiment, the cut-off value for the detection of a cancer is the average mean signal obtained when the immobilized antibody is incubated with samples from patients without the cancer. In general, a sample generating a signal that is three standard deviations above or below the predetermined cut-off value is considered positive for the cancer. For example, a reduced level of DACH1 protein or an additional marker downregulated by DACH1 may be indicative of the presence of cancer, or the metastatic potential of cancer. Similarly, a reduced level of DACH1 protein or an additional marker upregulated by DACH1 may be indicative of the presence of cancer, the stage of cancer, or the metastatic potential of cancer. In an alternate preferred embodiment, the cut-off value is determined using a Receiver Operator Curve, according to the method of Sackett et al., Clinical Epidemiology: A Basic Science for Clinical Medicine, Little Brown and Co., 1985, p. 106-7. Briefly, in this embodiment, the cut-off value may be determined from a plot of pairs of true positive rates (i.e., sensitivity) and false positive rates (100%-specificity) that correspond to each possible cut-off value for the diagnostic test result. The cut-off value on the plot that is the closest to the upper left-hand corner (i.e., the value that encloses the largest area) is the most accurate cut-off value, and a sample generating a signal that is higher than the cut-off value determined by this method may be considered positive. Alternatively, the cut-off value may be shifted to the left along the plot, to minimize the false positive rate, or to the right, to minimize the false negative rate. In general, a sample generating a signal that is higher than the cut-off value determined by this method is considered positive for a cancer. It will be understood that such embodiments can be applied where a decrease in the level of expression of a marker is used to detect cancer, or indicate progression of cancer.

In a related embodiment, the assay is performed in a flow-through or strip test format, wherein the binding agent is immobilized on a membrane, such as nitrocellulose. In the flow-through test, polypeptides within the sample bind to the immobilized binding agent as the sample passes through the membrane. A second, labeled binding agent then binds to the binding agent-polypeptide complex as a solution containing the second binding agent flows through the membrane. The detection of bound second binding agent may then be performed as described herein. In the strip test format, one end of the membrane to which binding agent is bound is immersed in a solution containing the sample. The sample migrates along the membrane through a region containing second binding agent and to the area of immobilized binding agent. The amount of immobilized antibody indicates the presence, stage, or metastatic potential of a cancer. Typically, the concentration of second binding agent at that site generates a pattern, such as a line, that can be read visually. In general, the amount of binding agent immobilized on the membrane is selected to generate a visually discernible pattern when the biological sample contains a level of polypeptide that would be sufficient to generate a positive signal in the two-antibody sandwich assay, in the format discussed above. Preferred binding agents for use in such assays are antibodies and antigen-binding fragments thereof Preferably, the amount of antibody immobilized on the membrane is from about 25 ng to about 1 μg, and more preferably from about 50 ng to about 500 ng. Such tests can typically be performed with a very small amount of biological sample.

Of course, numerous other assay protocols exist that are suitable for use with the markers described herein. The above descriptions are intended to be examples only. It will be apparent to those of ordinary skill in the art that the above protocols may be readily modified to use marker polypeptides to detect antibodies that bind to such polypeptides in a biological sample. The detection of such marker-specific antibodies may correlate with the presence of a cancer.

As noted herein, a cancer, or metastatic potential of cancer, may also, or alternatively, be detected based on the level of mRNA encoding DACH1 and, in some embodiments, an additional marker in a biological sample. For example, at least two oligonucleotide primers may be employed in a polymerase chain reaction (PCR) based assay to amplify a portion of a marker cDNA derived from a biological sample, wherein at least one of the oligonucleotide primers is specific for a polynucleotide encoding the marker. The amplified cDNA is then separated and detected using techniques well known in the art, such as gel electrophoresis. Similarly, oligonucleotide probes that specifically hybridize to a polynucleotide encoding a tumor protein may be used in a hybridization assay to detect the presence of polynucleotide encoding the tumor protein in a biological sample.

To permit hybridization under assay conditions, oligonucleotide primers and probes should comprise an oligonucleotide sequence that has at least about 60%, preferably at least about 75% and more preferably at least about 90% identity to a portion of a polynucleotide encoding a marker described herein that is at least 10 nucleotides, and preferably at least 20 nucleotides, in length. Preferably, oligonucleotide primers and/or probes hybridize to a polynucleotide encoding a polypeptide described herein under moderately stringent conditions, as defined above. Oligonucleotide primers and/or probes which may be usefully employed in the diagnostic methods described herein preferably are at least 10-40 nucleotides in length. In a preferred embodiment, the oligonucleotide primers comprise at least 10 contiguous nucleotides, more preferably at least 15 contiguous nucleotides, of a DNA molecule having a sequence as disclosed herein. Techniques for both PCR based assays and hybridization assays are well known in the art (see, for example, Mullis et al., Cold Spring Harbor Symp. Quant. Biol., 51:263, 1987; Erlich ed., PCR Technology, Stockton Press, NY, 1989). In some embodiments, the primers and probes may hybridize to DACH1 nucleic acids.

One embodiment employs RT-PCR, in which PCR is applied in conjunction with reverse transcription. Typically, RNA is extracted from a biological sample, such as biopsy tissue, and is reverse transcribed to produce cDNA molecules. PCR amplification using at least one specific primer generates a cDNA molecule, which may be separated and visualized using, for example, gel electrophoresis. Amplification may be performed on biological samples taken from a test patient and from an individual who is not afflicted with a cancer. The amplification reaction may be performed on several dilutions of cDNA spanning two orders of magnitude. A two-fold or greater change in expression in several dilutions of the test patient sample as compared to the same dilutions of the non-cancerous sample may typically considered positive.

In some embodiments, the methods and compositions described herein may be used to identify the progression of cancer. In such embodiments, assays as described herein for the diagnosis of a cancer may be performed over time, and the change in the level of reactive polypeptide(s) or polynucleotide(s) evaluated. For example, the assays may be performed every month for a period of from 6 months to 1 year, and thereafter performed as needed. In general, a cancer is progressing in those patients in whom the level of polypeptide or polynucleotide detected changes over time. For example, a cancer, such as breast cancer may be progressing where levels of expression of a marker such as DACH1 are decreasing, and/or levels of expression of markers such as Sox2, Klf4, and Nanog are increasing. In some embodiments, the level of expression of a marker can be used to determine the progression of a cancer.

Certain in vivo diagnostic assays may be performed directly on a tumor. One such assay involves contacting tumor cells with a binding agent, for example, an isolated antibody or fragment thereof, specific for DACH1. The bound binding agent may then be detected directly or indirectly via a reporter group. Such binding agents may also be used in histological applications. Alternatively, polynucleotide probes may be used within such applications.

As noted above, to improve sensitivity, multiple markers may be assayed within a given sample. Binding agents specific for different markers provided herein may be combined within a single assay. Further, multiple primers or probes may be used concurrently. The selection of markers may be based on routine experiments to determine combinations that results in optimal sensitivity. In addition, or alternatively, assays for tumor proteins provided herein may be combined with assays for other known tumor antigens.

In other aspects, cell capture technologies may be used prior to detection to improve the sensitivity of the various detection methodologies disclosed herein. Example cell enrichment methodologies employ immunomagnetic beads that are coated with specific monoclonal antibodies to surface cell markers, or tetrameric antibody complexes, may be used to first enrich or positively select cancer cells in a sample. Various commercially available kits may be used, including DYNABEADS® Epithelial Enrich (available from Life Technologies Corp., Carlsbad, Calif.), STEMSEP® (available from StemCell Technologies, Inc., Vancouver, BC), and ROSETTESEP (available from StemCell Technologies, Inc., Vancouver, BC). The skilled artisan will recognize that other readily available methodologies and kits may also be suitably employed to enrich or positively select desired cell populations.

DYNABEADS® Epithelial Enrich contains magnetic beads coated with monoclonal antibodies specific for two glycoprotein membrane antigens expressed on normal and neoplastic epithelial tissues. The coated beads may be added to a sample and the sample then applied to a magnet, thereby capturing the cells bound to the beads. The unwanted cells are washed away and the magnetically isolated cells eluted from the beads and used in further analyses. ROSETTESEP® can be used to enrich cells directly from a blood sample and consists of a cocktail of tetrameric antibodies that target a variety of unwanted cells and crosslinks them to glycophorin A on red blood cells (RBC) present in the sample, forming rosettes. When centrifuged over Ficoll, targeted cells pellet along with the free RBC.

Once a sample is enriched or positively selected, cells may be further analyzed. For example, the cells may be lysed and RNA isolated. RNA may then be subjected to RT-PCR analysis using breast tumor-specific primers in a Real-time PCR assay as described herein.

In some embodiments, cell capture technologies may be used in conjunction with real-time PCR to provide a more sensitive tool for measuring the levels of expression of markers in cancer cells. Detection of breast cancer cells in bone marrow samples, peripheral blood, biopsies, and other samples is desirable for diagnosis and prognosis in breast cancer patients.

Some embodiments include making and using antibodies and fragments thereof specific to DACH1 protein. Methods of making polyclonal and monoclonal antibodies are well known. For example, monoclonal antibodies to epitopes of DACH1 can be prepared from murine hybridomas according to the classical method of Kohler, G. and Milstein, C., Nature 256:495 (1975) or any of the well-known derivative methods thereof.

In addition, antibody fragment preparations prepared from the produced antibodies are contemplated. “Antibody fragments” comprise a portion of an intact antibody, preferably the antigen binding or variable region of the intact antibody. Examples of antibody fragments include Fab, Fab′, F(ab′)₂, and Fv fragments; diabodies; linear antibodies (Zapata et al., Protein Eng. 8(10): 1057-1062 [1995]); single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)₂ fragment that has two antigen-combining sites and is still capable of cross-linking antigen.

“Fv” is the minimum antibody fragment which contains a complete antigen-recognition and -binding site. This region consists of a dimer of one heavy- and one light-chain variable domain in tight, non-covalent association. It is in this configuration that the three Complementarity Determining Regions (CDRs) of each variable domain interact to define an antigen-binding site on the surface of the V_{H-VL} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment also contains the constant domain of the light chain and the first constant domain (CH1) of the heavy chain. Fab fragments differ from Fab′ fragments by the addition of a few residues at the carboxy terminus of the heavy chain CH1 domain including one or more cysteines from the antibody hinge region.

### Pharmaceutical Compositions

Some embodiments relate to compositions capable of treating or ameliorating a cancer, such as breast cancer, or other disorders relating to neuron or hematological diseases. In some embodiments, treating or ameliorating cancer can include increasing the levels of markers such as DACH1 in the cell of a subject. In some embodiments, a composition can include a nucleic acid encoding at least a portion of the DACH1 polypeptide. At least a portion as used herein can refer to at least about 5%, 10%, 20%, 50%, 70%, 80%, 90%, 95%, 99%, or 100%. In some embodiments, a composition can comprise a polypeptide comprising the sequence of DACH1 or fragment thereof. A “therapeutically effective amount” is a quantity of a chemical composition (such as a nucleic acid construct, vector, or polypeptide) used to achieve a desired effect in a subject being treated.

In some embodiments, a pharmaceutical composition can include a nucleic acid encoding at least a portion of DACH1 operably linked to a regulatory sequence. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990), the disclosure of which is incorporated herein by reference in its entirety. Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like.

In some circumstances, it may be desirable to regulate expression of a transgene in a gene therapy vector. For example, different viral promoters with varying strengths of activity may be utilized depending on the level of expression desired. In mammalian cells, the CMV immediate early promoter is often used to provide strong transcriptional activation. Modified versions of the CMV promoter that are less potent have also been used when reduced levels of expression of the transgene are desired. When expression of a transgene in hematopoetic cells is desired, retroviral promoters such as the LTRs from MLV or are often used. Other viral promoters that may be used depending on the desired effect include SV40, RSV LTR, HIV-1 and HfV-2 LTR, adenovirus promoters such as from the E1A, E2A, or MLP region, AAV LTR, cauliflower mosaic virus, HSV-TK, and avian sarcoma virus.

Similarly tissue specific promoters may be used to effect transcription in specific tissues or cells so as to reduce potential toxicity or undesirable effects to non-targeted tissues. In some embodiments, a tissue-specific promoter can include a mammary-specific promoter. Examples of mammary-specific promoters include adipose differentiation related protein promoter, whey acidic protein promoter, β casein promoter, lactalbumin promoter and β-lactoglobulin promoter (Li et al., 1998, Oncogene 16:997-1007; Oh et al., 1999, Transgenic Res 8:307-11; Brandt et al., 2000, Oncogene 19:2129-37, incorporated by reference in their entireties). More examples of tissue-specific promoters include promoters such as the PSA, probasin, prostatic acid phosphatase or prostate-specific glandular kallikrein (hK2) may be used to target gene expression in the prostate. Similarly, promoters as follows may be used to target gene expression in other tissues.

More tissue specific promoters include in (a) pancreas: insulin, elastin, amylase, pdr-I, pdx-I, glucokinase; (b) liver: albumin PEPCK, HBV enhancer, alpha fetoprotein, apolipoprotein C, alpha-I antitrypsin, vitellogenin, NF-AB, Transthyretin; (c) skeletal muscle: myosin H chain, muscle creatine kinase, dystrophin, calpain p94, skeletal alpha-actin, fast troponin 1; (d) skin: keratin K6, keratin KI; (e) lung: CFTR, human cytokeratin IS (K 18), pulmonary surfactant proteins A, B and C, CC-10, Pi; (f) smooth muscle: sm22 alpha, SM-alpha-actin; (g) endothelium: endothelin- I, E-selectin, von Willebrand factor, TIE (Korhonen et al., 1995), KDR/flk-I; (h) melanocytes: tyrosinase; (i) adipose tissue: lipoprotein lipase (Zechner et al., 1988), adipsin (Spiegelman et al., 1989), acetyl-CoA carboxylase (Pape and Kim, 1989), glycerophosphate dehydrogenase (Dani et al., 1989), adipocyte P2 (Hunt et al., 1986); and (j) blood: P-globin.

In certain embodiments, it may be desirable to activate transcription at specific times after administration of the gene therapy vector. This may be done with such promoters as those that are hormone or cytokine regulatable. For example in gene therapy applications where the indication is in a gonadal tissue where specific steroids are produced or routed to, use of androgen or estrogen regulated promoters may be advantageous. Such promoters that are hormone regulatable include MMTV, MT-1, ecdysone and RuBisco. Other hormone regulated promoters such as those responsive to thyroid, pituitary and adrenal hormones are expected to be useful with the nucleic acids described herein. Cytokine and inflammatory protein responsive promoters that could be used include K and T Kininogen (Kageyama et al., 1987), c-fos, TNF-alpha, C-reactive protein (Arcone et al., 1988), haptoglobin (Oliviero et al., 1987), serum amyloid A2, C/EBP alpha, IL-1, IL-6 (Poli and Cortese, 1989), Complement C3 (Wilson et al., 1990), IL-8, alpha-1 acid glycoprotein (Prowse and Baumann, 1988), alpha-1 antitypsin, lipoprotein lipase (Zechner et al., 1988), angiotensinogen (Ron et al., 1991), fibrinogen, c-jun (inducible by phorbol esters, TNF alpha, UV radiation, retinoic acid, and hydrogen peroxide), collagenase (induced by phorbol esters and retinoic acid), metallothionein (heavy metal and glucocorticoid inducible), Stromelysin (inducible by phorbol ester, interleukin-1 and EGF), alpha-2 macroglobulin and alpha-I antichymotrypsin.

In some embodiments, it is envisioned that cell cycle regulatable promoters may be useful. For example, in a bi-cistronic gene therapy vector, use of a strong CMV promoter to drive expression of a first gene such as p16 that arrests cells in the G1 phase could be followed by expression of a second gene such as p53 under the control of a promoter that is active in the G1 phase of the cell cycle, thus providing a “second hit” that would push the cell into apoptosis. Other promoters such as those of various cyclins, PCNA, galectin-3, E2FI, p53 and BRCAI could be used.

It is envisioned that any of the promoters described herein, alone or in combination with another, may be useful depending on the action desired.

In addition, the promoters described herein should not be considered to be exhaustive or limiting, those of skill in the art will know of other promoters that may be used in conjunction with the nucleic acids and methods disclosed herein.

In some embodiments, the nucleic acids for producing or administering any of the DACH1 polypeptides described herein may contain one or more enhancers. Enhancers are genetic elements that increase transcription from a promoter located at a distant position on the same molecule of DNA. Enhancers are organized much like promoters. That is, they are composed of many individual elements, each of which binds to one or more transcriptional proteins. The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Nucleic acid constructs encoding any DACH1 polypeptide described herein can be introduced in vivo as naked DNA plasmids. DNA vectors can be introduced into the desired host cells by methods known in the art, including but not limited to transfection, electroporation (e.g., transcutaneous electroporation), microinjection, transduction, cell fusion, DEAE dextran, calcium phosphate precipitation, use of a gene gun, or use of a DNA vector transporter (See e.g., Wu et al. J. Biol. Chem., 267:963-967, 1992; Wu and Wu J. Biol. Chem., 263:14621-14624, 1988; and Williams et al. Proc. Natl. Acad. Sci. USA 88:2726-2730, 1991). A needleless delivery device, such as a BIOJECTOR® needleless injection device can be utilized to introduce the therapeutic nucleic acid constructs in vivo. Receptor-mediated DNA delivery approaches can also be used (Curiel et al. Hum. Gene Ther., 3:147-154, 1992; and Wu and Wu, J. Biol. Chem., 262:4429-4432, 1987). Methods for formulating and administering naked DNA to mammalian muscle tissue are disclosed in U.S. Pat. Nos. 5,580,859 and 5,589,466, both of which are herein incorporated by reference in their entireties. Other molecules are also useful for facilitating transfection of a nucleic acid in vivo, such as a cationic oligopeptide (e.g., WO95/21931), peptides derived from DNA binding proteins (e.g., WO96/25508), or a cationic polymer (e.g., WO95/21931), the disclosures of which are incorporated herein by reference in their entireties.

Alternatively, electroporation can be utilized conveniently to introduce nucleic acid constructs encoding any DACH1 polypeptide described herein into cells. Electroporation is well known by those of ordinary skill in the art (see, for example: Lohr et al. Cancer Res. 61:3281-3284, 2001; Nakano et al. Hum Gene Ther. 12:1289-1297, 2001; Kim et al. Gene Ther. 10:1216-1224, 2003; Dean et al. Gene Ther. 10:1608-1615, 2003; and Young et al. Gene Ther 10:1465-1470, 2003). For example, in electroporation, a high concentration of vector DNA is added to a suspension of host cell (such as isolated autologous peripheral blood or bone marrow cells) and the mixture shocked with an electrical field. Transcutaneous electroporation can be utilized in animals and humans to introduce heterologous nucleic acids into cells of solid tissues (such as muscle) in vivo. Typically, the nucleic acid constructs are introduced into tissues in vivo by introducing a solution containing the DNA into a target tissue, for example, using a needle or trochar in conjunction with electrodes for delivering one or more electrical pulses. For example, a series of electrical pulses can be utilized to optimize transfection, for example, between 3 and ten pulses of 100 V and 50 msec. In some cases, multiple sessions or administrations are performed.

Another well known method that can be used to introduce nucleic acid constructs encoding any DACH1 polypeptide described herein into host cells is particle bombardment (also know as biolistic transformation). Biolistic transformation is commonly accomplished in one of several ways. One common method involves propelling inert or biologically active particles at cells. This technique is disclosed in, e.g., U.S. Pat. Nos. 4,945,050, 5,036,006; and 5,100,792, all to Sanford et al., the disclosures of which are hereby incorporated by reference in their entireties. Generally, this procedure involves propelling inert or biologically active particles at the cells under conditions effective to penetrate the outer surface of the cell and to be incorporated within the interior thereof. When inert particles are utilized, the plasmid can be introduced into the cell by coating the particles with the plasmid containing the exogenous DNA. Alternatively, the target cell can be surrounded by the plasmid so that the plasmid is carried into the cell by the wake of the particle.

Alternatively, the vector can be introduced in vivo by lipofection. For the past decade, there has been increasing use of liposomes for encapsulation and transfection of nucleic acids in vitro. Synthetic cationic lipids designed to limit the difficulties and dangers encountered with liposome mediated transfection can be used to prepare liposomes for in vivo transfection of a gene encoding a marker (Felgner et. al. Proc. Natl. Acad. Sci. USA 84:7413-7417, 1987; Mackey, et al. Proc. Natl. Acad. Sci. USA 85:8027-8031, 1988; Ulmer et al. Science 259:1745-1748, 1993, the disclosures of which are incorporated herein by reference in their entireties). The use of cationic lipids can promote encapsulation of negatively charged nucleic acids, and also promote fusion with negatively charged cell membranes (Felgner and Ringold Science 337:387-388, 1989, the disclosure of which is incorporated by reference herein in its entirety). Particularly useful lipid compounds and compositions for transfer of nucleic acids are described in WO95/18863 and WO96/17823, and in U.S. Pat. No. 5,459,127, the disclosures of which are incorporated herein by reference in their entireties.

In some embodiments, the nucleic acid constructs encoding any DACH1 polypeptide described herein are viral vectors. Methods for constructing and using viral vectors are known in the art (See e.g., Miller and Rosman, BioTech., 7:980-990, 1992). Preferably, the viral vectors are replication defective, that is, they are unable to replicate autonomously in the target cell. In general, the genome of the replication defective viral vectors that are used within the scope of the present disclosure lack at least one region that is necessary for the replication of the virus in the infected cell. These regions can either be eliminated (in whole or in part), or be rendered non-functional by any technique known to a person skilled in the art. These techniques include the total removal, substitution (by other sequences, in particular by the inserted nucleic acid), partial deletion or addition of one or more bases to an essential (for replication) region. Such techniques can be performed in vitro (for example, on the isolated DNA).

In some cases, the replication defective virus retains the sequences of its genome that are necessary for encapsidating the viral particles. DNA viral vectors commonly include attenuated or defective DNA viruses, including, but not limited to, herpes simplex virus (HSV), papillomavirus, Epstein Barr virus (EBV), adenovirus, adeno-associated virus (AAV), Moloney leukemia virus (MLV) and human immunodeficiency virus (HIV) and the like. Defective viruses, that entirely or almost entirely lack viral genes, are preferred, as defective virus is not infective after introduction into a cell. Use of defective viral vectors allows for administration to cells in a specific, localized area, without concern that the vector can infect other cells. Thus, a specific tissue can be specifically targeted. Examples of particular vectors include, but are not limited to, a defective herpes virus 1 (HSV1) vector (Kaplitt et al. Mol. Cell. Neurosci., 2:320-330, 1991, the disclosure of which is incorporated herein by reference in its entirety), defective herpes virus vector lacking a glycoprotein L gene (See for example, Patent Publication RD 371005 A, the disclosure of which is incorporated herein by reference in its entirety), or other defective herpes virus vectors (See e.g., WO 94/21807; and WO 92/05263, the disclosures of which are incorporated herein by reference in their entireties); an attenuated adenovirus vector, such as the vector described by Stratford-Perricaudet et al. (J. Clin. Invest., 90:626-630 1992; La Salle et al., Science 259:988-990, 1993, the disclosure of which is incorporated herein by reference in its entirety); and a defective adeno-associated virus vector (Samulski et al., J. Virol., 61:3096-3101, 1987; Samulski et al., J. Virol., 63:3822-3828, 1989; and Lebkowski et al., Mol. Cell. Biol., 8:3988-3996, 1988, the disclosures of which are incorporated herein by reference in their entireties).

In some embodiments, the vectors encoding any DACH1 polypeptide described herein may be adenovirus vectors. Adenoviruses are eukaryotic DNA viruses that can be modified to efficiently deliver a nucleic acid of the disclosure to a variety of cell types. Various serotypes of adenovirus exist. Of these serotypes, preference is given, within the scope of the present disclosure, to type 2, type 5 or type 26 human adenoviruses (Ad 2 or Ad 5), or adenoviruses of animal origin (See e.g., WO94/26914 and WO2006/020071, the disclosures of which are incorporated herein by reference in their entireties). Those adenoviruses of animal origin that can be used within the scope of the present disclosure include adenoviruses of canine, bovine, murine (e.g., Mav1, Beard et al. Virol., 75-81, 1990, the disclosure of which is incorporated herein by reference in its entirety), ovine, porcine, avian, and simian (e.g., SAV) origin. In some embodiments, the adenovirus of animal origin is a canine adenovirus, such as a CAV2 adenovirus (e.g., Manhattan or A26/61 strain (ATCC VR-800)).

The replication defective adenoviral vectors may include the ITRs, an encapsidation sequence and the polynucleotide sequence of interest. In some embodiments, at least the E1 region of the adenoviral vector is non-functional. The deletion in the E1 region preferably extends from nucleotides 455 to 3329 in the sequence of the Ad5 adenovirus (PvuII-BglII fragment) or 382 to 3446 (HinfII-Sau3A fragment). Other regions can also be modified, in particular the E3 region (e.g., WO95/02697, the disclosure of which is incorporated herein by reference in its entirety), the E2 region (e.g., WO94/28938, the disclosure of which is incorporated herein by reference in its entirety), the E4 region (e.g., WO94/28152, WO94/12649 and WO95/02697, the disclosures of which are incorporated herein by reference in their entireties), or in any of the late genes L1-L5.

In other embodiments, the adenoviral vector has a deletion in the E1 region (Ad 1.0). Examples of E1-deleted adenoviruses are disclosed in EP 185,573, the contents of which are incorporated herein by reference. In another embodiment, the adenoviral vector has a deletion in the E1 and E4 regions (Ad 3.0). Examples of E1/E4-deleted adenoviruses are disclosed in WO95/02697 and WO96/22378, the disclosures of which are incorporated herein by reference in their entireties.

The replication defective recombinant adenoviruses can be prepared by any technique known to the person skilled in the art (See e.g., Levrero et al. Gene 101:195, 1991; EP 185 573; and Graham EMBO J., 3:2917, 1984, the disclosures of which are incorporated herein by reference in their entireties). In particular, they can be prepared by homologous recombination between an adenovirus and a plasmid, which includes, inter alia, the DNA sequence of interest. The homologous recombination is accomplished following co-transfection of the adenovirus and plasmid into an appropriate cell line. The cell line that is employed should preferably (i) be transformable by the elements to be used, and (ii) contain the sequences that are able to complement the part of the genome of the replication defective adenovirus, preferably in integrated form in order to avoid the risks of recombination. Examples of cell lines that can be used are the human embryonic kidney cell line 293 (Graham et al. J. Gen. Virol. 36:59, 1977, the disclosure of which is incorporated herein by reference in its entirety), which contains the left-hand portion of the genome of an Ad5 adenovirus (12%) integrated into its genome, and cell lines that are able to complement the E1 and E4 functions, as described in applications WO94/26914 and WO95/02697, the disclosures of which are incorporated herein by reference in their entireties. Recombinant adenoviruses are recovered and purified using standard molecular biological techniques that are well known to one of ordinary skill in the art.

In some embodiments, pharmaceutical compositions described herein comprise at least a portion of the DACH1 protein. In some embodiments, the DACH1 protein can be administered to a subject. In some embodiments, a portion of the DACH1 protein can be administered to a subject, where the portion contains biological activity useful to treat or ameliorate cancer. Methods to map regions in the DACH1 polypeptide with specific activity are well known and include techniques such as deletion analysis and mutagenesis analysis.

Some embodiments include pharmaceutical compositions comprising suitable carriers. While any suitable carrier known to those of ordinary skill in the art may be employed in the pharmaceutical compositions described herein, the type of carrier will typically vary depending on the mode of administration. Compositions described herein may be formulated for any appropriate manner of administration, including for example, topical, oral, nasal, mucosal, intravenous, intracranial, intraperitoneal, subcutaneous and intramuscular administration.

Carriers for use within such pharmaceutical compositions are biocompatible, and may also be biodegradable. In certain embodiments, the formulation preferably provides a relatively constant level of active component release. In other embodiments, however, a more rapid rate of release immediately upon administration may be desired. The formulation of such compositions is well within the level of ordinary skill in the art using known techniques. Illustrative carriers useful in this regard include microparticles of poly(lactide-co-glycolide), polyacrylate, latex, starch, cellulose, dextran and the like. Other illustrative delayed-release carriers include supramolecular biovectors, which comprise a non-liquid hydrophilic core (e.g., a cross-linked polysaccharide or oligosaccharide) and, optionally, an external layer comprising an amphiphilic compound, such as a phospholipid (see e.g., U.S. Pat. No. 5,151,254 and PCT Publication Nos. WO94/20078, WO/94/23701 and WO96/06638). The amount of active compound contained within a sustained release formulation depends upon the site of implantation, the rate and expected duration of release and the nature of the condition to be treated or prevented.

In another illustrative embodiment, biodegradable microspheres (e.g., poly(actate polyglycolate) are employed as carriers for the compositions described herein. Suitable biodegradable microspheres are disclosed, for example, in U.S. Pat. Nos. 4,897,268; 5,075,109; 5,928,647; 5,811,128; 5,820,883; 5,853,763; 5,814,344, 5,407,609 and 5,942,252. Modified hepatitis B core protein carrier systems such as described in PCT Publication No. WO/9940934, and references cited therein, will also be useful for many applications. Another illustrative carrier/delivery system employs a carrier comprising particulate-protein complexes, such as those described in U.S. Pat. No. 5,928,647, which are capable of inducing a class I-restricted cytotoxic T lymphocyte responses in a host.

The pharmaceutical compositions described herein will often further comprise one or more buffers (e.g., neutral buffered saline or phosphate buffered saline), carbohydrates (e.g., glucose, mannose, sucrose or dextrans), mannitol, proteins, polypeptides or amino acids such as glycine, antioxidants, bacteriostats, chelating agents such as EDTA or glutathione, adjuvants (e.g., aluminum hydroxide), solutes that render the formulation isotonic, hypotonic or weakly hypertonic with the blood of a recipient, suspending agents, thickening agents and/or preservatives. Alternatively, compositions described herein may be formulated as a lyophilizate.

The pharmaceutical compositions described herein may be presented in unit-dose or multi-dose containers, such as sealed ampoules or vials. Such containers are typically sealed in such a way to preserve the sterility and stability of the formulation until use. In general, formulations may be stored as suspensions, solutions or emulsions in oily or aqueous vehicles. Alternatively, a pharmaceutical composition may be stored in a freeze-dried condition requiring only the addition of a sterile liquid carrier immediately prior to use.

The development of suitable dosing and treatment regimens for using the particular compositions described herein in a variety of treatment regimens, including e.g., oral, parenteral, intravenous, intranasal, and intramuscular administration and formulation, is well known in the art, some of which are briefly discussed below for general purposes of illustration.

In certain applications, the pharmaceutical compositions described herein may be delivered via oral administration to an animal. As such, these compositions may be formulated with an inert diluent or with an assimilable edible carrier, or they may be enclosed in hard- or soft-shell gelatin capsule, or they may be compressed into tablets, or they may be incorporated directly with the food of the diet. The active compounds may even be incorporated with excipients and used in the form of ingestible tablets, buccal tables, troches, capsules, elixirs, suspensions, syrups, wafers, and the like (see, for example, Mathiowitz et al., Nature 1997 Mar. 27; 386(6623):410-4; Hwang et al., Crit. Rev Ther Drug Carrier Syst 1998; 15(3):243-84; U.S. Pat. No. 5,641,515; U.S. Pat. No. 5,580,579 and U.S. Pat. No. 5,792,451). Tablets, troches, pills, capsules and the like may also contain any of a variety of additional components, for example, a binder, such as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. Of course, any material used in preparing any dosage unit form should be pharmaceutically pure and substantially non-toxic in the amounts employed. In addition, the active compounds may be incorporated into sustained-release preparation and formulations.

Typically, these formulations will contain at least about 0.1% of the active compound or more, although the percentage of the active ingredient(s) may, of course, be varied and may conveniently be between about 1 or 2% and about 60% or 70% or more of the weight or volume of the total formulation. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

For oral administration the compositions of the preferred embodiments may alternatively be incorporated with one or more excipients in the form of a mouthwash, dentifrice, buccal tablet, oral spray, or sublingual orally-administered formulation. Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

In certain circumstances it will be desirable to deliver the pharmaceutical compositions disclosed herein parenterally, intravenously, intramuscularly, or even intraperitoneally. Such approaches are well known to the skilled artisan, some of which are further described, for example, in U.S. Pat. No. 5,543,158; U.S. Pat. No. 5,641,515 and U.S. Pat. No. 5,399,363. In certain embodiments, solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations generally will contain a preservative to prevent the growth of microorganisms.

Illustrative pharmaceutical forms suitable for injectable use include sterile aqueous solutions or dispersions and sterile powders for the extemporaneous preparation of sterile injectable solutions or dispersions (for example, see U.S. Pat. No. 5,466,468). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and/or by the use of surfactants. The prevention of the action of microorganisms can be facilitated by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

In one embodiment, for parenteral administration in an aqueous solution, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. Moreover, for human administration, preparations will of course preferably meet sterility, pyrogenicity, and the general safety and purity standards as required by FDA Office of Biologics standards.

In another embodiment, the compositions disclosed herein may be formulated in a neutral or salt form. Illustrative pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective.

The carriers can further comprise any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions. The phrase “pharmaceutically-acceptable” refers to molecular entities and compositions that do not produce an allergic or similar untoward reaction when administered to a human.

In certain embodiments, the pharmaceutical compositions may be delivered by intranasal sprays, inhalation, and/or other aerosol delivery vehicles. Methods for delivering genes, nucleic acids, and peptide compositions directly to the lungs via nasal aerosol sprays has been described, e.g., in U.S. Pat. No. 5,756,353 and U.S. Pat. No. 5,804,212. Likewise, the delivery of drugs using intranasal microparticle resins (Takenaga et al., J Controlled Release 1998 Mar. 2; 52(1-2):81-7) and lysophosphatidyl-glycerol compounds (U.S. Pat. No. 5,725,871) are also well-known in the pharmaceutical arts. Likewise, illustrative transmucosal drug delivery in the form of a polytetrafluoroetheylene support matrix is described in U.S. Pat. No. 5,780,045.

In certain embodiments, liposomes, nanocapsules, microparticles, lipid particles, vesicles, and the like, are used for the introduction of the compositions of the preferred embodiments into suitable host cells/organisms. In particular, the compositions may be formulated for delivery either encapsulated in a lipid particle, a liposome, a vesicle, a nanosphere, or a nanoparticle or the like. Alternatively, compositions can be bound, either covalently or non-covalently, to the surface of such carrier vehicles.

The formation and use of liposome and liposome-like preparations as potential drug carriers is generally known to those of skill in the art (see for example, Lasic, Trends Biotechnol 1998 July; 16(7):307-21; Takakura, Nippon Rinsho 1998 March; 56(3):691-5; Chandran et al., Indian J Exp Biol. 1997 August; 35(8):801-9; Margalit, Crit Rev Ther Drug Carrier Syst. 1995; 12(2-3):233-61; U.S. Pat. No. 5,567,434; U.S. Pat. No. 5,552,157; U.S. Pat. No. 5,565,213; U.S. Pat. No. 5,738,868 and U.S. Pat. No. 5,795,587, each specifically incorporated herein by reference in its entirety).

Liposomes have been used successfully with a number of cell types that are normally difficult to transfect by other procedures, including T cell suspensions, primary hepatocyte cultures and PC 12 cells (Renneisen et al., J Biol Chem. 1990 Sep. 25; 265(27):16337-42; Muller et al., DNA Cell Biol. 1990 April; 9(3):221-9). In addition, liposomes are free of the DNA length constraints that are typical of viral-based delivery systems. Liposomes have been used effectively to introduce genes, various drugs, radiotherapeutic agents, enzymes, viruses, transcription factors, allosteric effectors and the like, into a variety of cultured cell lines and animals. Furthermore, the use of liposomes does not appear to be associated with autoimmune responses or unacceptable toxicity after systemic delivery. In certain embodiments, liposomes are formed from phospholipids that are dispersed in an aqueous medium and spontaneously form multilamellar concentric bilayer vesicles (also termed multilamellar vesicles (MLVs).

Alternatively, in other embodiments, pharmaceutically-acceptable nanocapsule formulations of the compositions are provided. Nanocapsules can generally entrap compounds in a stable and reproducible way (see, for example, Quintanar-Guerrero et al., Drug Dev Ind Pharm. 1998 December; 24(12):1113-28). To avoid side effects due to intracellular polymeric overloading, such ultrafine particles (sized around 0.1 μm) may be designed using polymers able to be degraded in vivo. Such particles can be made as described, for example, by Couvreur et al., Crit Rev Ther Drug Carrier Syst. 1988; 5(1):1-20; zur Muhlen et al., Eur J Pharm Biopharm. 1998 March; 45(2):149-55; Zambaux et al. J Controlled Release. 1998 Jan. 2; 50(1-3):31-40; and U.S. Pat. No. 5,145,684.

In further aspects, the pharmaceutical compositions described herein may be used for the treatment of cancer, particularly for the treatment of breast cancer. Within such methods, the pharmaceutical compositions described herein are administered to a patient, typically a warm-blooded animal, preferably a human. A patient may or may not be afflicted with cancer. Accordingly, the pharmaceutical compositions described herein may be used to prevent the development of a cancer or to treat a patient afflicted with a cancer. Pharmaceutical compositions may be administered either prior to or following surgical removal of primary tumors and/or treatment such as administration of radiotherapy or conventional chemotherapeutic drugs. As discussed herein, administration of the pharmaceutical compositions may be by any suitable method, including administration by intravenous, intraperitoneal, intramuscular, subcutaneous, intranasal, intradermal, anal, vaginal, topical and oral routes.

### Kits

Some embodiments relate to kits for use with any of the diagnostic methods described herein. Such kits typically comprise two or more components necessary for performing a diagnostic assay. Components may be compounds, reagents, containers and/or equipment. For example, one container within a kit may contain an antibody or fragment thereof that specifically binds to DACH1. Such antibodies or fragments may be provided attached to a support material, as described herein. One or more additional containers may enclose elements, such as reagents or buffers, to be used in the assay. Such kits may also, or alternatively, contain a detection reagent as described above that contains a reporter group suitable for direct or indirect detection of antibody binding.

Alternatively, a kit may be designed to detect the level of mRNA encoding a tumor protein in a biological sample. Such kits generally comprise at least one oligonucleotide probe or primer, as described above, that hybridizes to a polynucleotide encoding a tumor protein. Such an oligonucleotide may be used, for example, within a PCR or hybridization assay. Additional components that may be present within such kits include a second oligonucleotide and/or a diagnostic reagent or container to facilitate the detection of a polynucleotide encoding a marker.

In some embodiments kits can be used to diagnose the presence of a cancer, the stage of progression of a cancer, or the metastatic potential of a cancer. In some embodiments, the cancer can comprise breast cancer, skin cancer, ovarian cancer, kidney cancer, lung cancer, brain cancer, endometrial cancer, pancreatic cancer or prostate cancer.

### Certain Methods for Identifying Agents

More embodiments include methods of identifying compounds and agents useful for the methods and compositions described herein. Some such methods can be useful to evaluate test compounds useful to treat disorders related to decreased expression of DACH1.

In some embodiments, a test compound is evaluated by contacting the cell with the test compound. A test compound that increases the level of DACH1 protein or the level of a nucleic acid encoding DACH1 may be useful to treat certain cancers and/or tumors, e.g. breast cancer, and cancer stem cells. More methods include comparing the level of a nucleic acid encoding DACH1 or the level of DACH1 protein in a target cell to the level of a nucleic acid encoding DACH1 or the level of DACH1 protein in a target cell contacted with the test compound. More methods can also include selecting a test compound that reduces the level of a nucleic acid encoding Sox2, Nanog, and/or Klf4, or reduces the level of Sox2, Nanog, and/or Klf4 protein in a target cell.

Some embodiments include screening for compounds that increase expression levels of DACH1. Some such compounds and agents may be useful to increase growth and proliferation of certain stem cells. Some methods include contacting a cell with a test agent. An increase in DACH1 expression in response to contacting the cell with the compound or agent can be indicative that the compound or agent may be useful to increase the growth and/or proliferation of stem cells.

### Certain Embodiments for Diagnosis and Prognosis of Disorders

Some embodiments include diagnosis and/or prognosis of particular disorders, e.g., cancers such as breast cancer. In some such embodiments, the expression of genes associated with the retinal determination gene network (RDGN) e.g. DACH1, Six1 and Eya1, and the RDGN can indicate a disorder and/or the progression of a disorder.

The RDGN is important for the development of many organs or tissues and is involved in human cancer. The expression of DACH1 is lost in breast cancer; ectopic expression of DACH1 inhibited breast cancer cellular proliferation in vitro and tumor generation in vivo. Reduced DACH1 expression can predict 40-month worse survival in human breast cancer. Reduced DACH1 expression is associated with decreased 5-year survival in endometrial cancer. Over-expression of Six1 and Eya1 can be found in many kinds of human cancer.

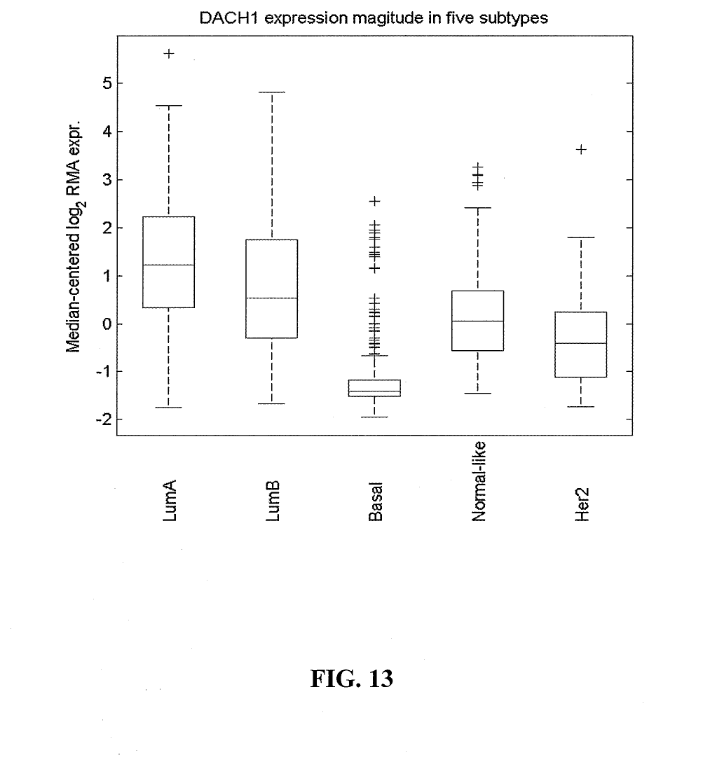

Data provided herein demonstrates that DACH1 mRNA expression was selectively decreased in basal-like breast cancer, correspondingly protein abundance of DACH1 was also reduced in basal-like breast cancer. Human breast cancer can be classified into five distinct genotypes based on molecular genetic profiling. Dach1 mRNA abundance is selectively reduced in the basal phenotype (FIG. 13). The basal or triple negative phenotype does not respond to current estrogen inhibitors or ErbB2 inhibitors (herceptin). Moreover, treatment response and prognosis of basal-like cancer is different from other breast cancer subtypes. Basal-like breast cancer has cancer stem cell properties and is a more aggressive type of cancer. New therapies are required for basal/triple negative breast cancer.

Certain embodiments include screening for new therapies of basal/triple negative breast cancer using expression levels of DACH1 in such tumors as a marker for the efficacy of certain putative therapies. Certain embodiments include selecting a therapy to treat a cancer, such as breast cancer by determining the level of DACH1 gene expression or protein expression in a patient.

### Certain Embodiments Include Use of DNA-Methyltransferase Inhibitors

As described herein, the DACH1 promoter is methylated in breast cancer cell lines and treatment of different breast cancer cell lines with 5-aza-2′-deoxycytidine induced DACH1 mRNA expression. Reactivation of silenced tumor suppressor genes by DNA-methyltransferase inhibitors (DMTIs) such as 5-azacytidine (Vidaza) and its congener 5-aza-2′-deoxycytidine (Decitabine), can provide an alternate approach to cancer therapy. Both drugs have been approved by FDA for clinical treatment of conditions such as myelodysplastic syndrome. As DACH1 promoter methylation correlated with transcriptional silencing, which was reversible with the methylation inhibitor 5-aza-2′-deoxycytidine, DNA-methyltransferase inhibitors for DACH1 negative tumors provide an alternate treatment for patients with tumors that have reduced or no DACH1 expression

### Certain Embodiments Include Treatment of Cancer Stem Cells

Cancer stem cells contribute to tumor initiation, progression and therapy resistance. DACH1 inhibits breast cancer stem cells. DACH1 may regulate cancer stem cells in other tumor types (prostate, kidney, lung and other tumors). Certain embodiments provided herein include the treatment of cancer stem cells. Cancer stem cells may be present in a variety of tumors, e.g., tumors derived from breast, prostate, kidney, and lung, etc. Some embodiments provided herein include the use of compositions provided herein to treat such cancer stem cells.

### Certain Embodiments Include Use of Anti-IL8 Therapies

Loss of DACH in tumors increases IL8 expression and secretion. IL8 is repressed by DACH and IL8 antibodies block breast cancer metastasis. Certain embodiments include use of therapies to reduce and/or block IL8 expression in tumors with reduced DACH1 expression. Such anti-IL8 therapies can include, for example, anti-IL8 drugs, immunoneutralizing antibodies or other anti IL8 treatment.

### Certain Embodiments Include Treating Disorders Associated With the Forkhead Family of Proteins

DACH1 has DNA sequence specific binding ability and identified that DACH1 competed with forkhead proteins and their activity and function (Zhou J, et al. (2010) PNAS 107: 6864-6869). Forkhead proteins are well known in diverse biological processes and regulate stem cells, cancer and human diseases. FOX proteins include Forkhead box-containing proteins. The FOX proteins are a family of evolutionarily conserved transcription regulators involved in diverse biological processes (Myatt S S, Lam E W(2007) The emerging roles of forkhead box (Fox) proteins in cancer. Nat Rev Cancer 7:847-859). FOX protein function can either promote or inhibit tumorigenesis, and deregulation of FOX protein function in human tumorigenesis may occur by alteration in upstream regulators or genetic events such as mutations of the DNA binding domain, or translocations, which often disrupt the DNA binding domain (Arden K C (2007) FoxOs in tumor suppression and stem cell maintenance. Cell 128:235-237) FOXM1 is overexpressed in human tumors (Kalinichenko V V, et al. (2004) Foxm1b transcription factor is essential for development of hepatocellular carcinomas and is negatively regulated by the p19ARF tumor suppressor. Genes Dev 18:830-850) and promotes tumor growth (Kim I M, et al. (2006) The Forkhead Box m1 transcription factor stimulates the proliferation of tumor cells during development of lung cancer. Cancer Res 66:2153-2161). Inhibition of FOXM1 expression reduces growth of murine tumors in response to carcinogens, and diminishes DNA replication and mitosis of tumor cells (Wang I C, et al. (2005) Forkhead box M1 regulates the transcriptional network of genes essential for mitotic progression and genes encoding the SCF (Skp2-Cksl) ubiquitin ligase). FOXC2, associated with aggressive basal-like breast cancer, enhances tumor metastasis and invasion (Mani S A, et al. (2007) Mesenchyme Forkhead 1 (FOXC2) plays a key role in metastasis and is associated with aggressive basal-like breast cancers. Proc Natl Acad Sci USA 104:10069-10074). Certain embodiments include selecting treatment for disorders associated with abnormal expression and or function of Forkhead proteins by determining expression levels of DACH1.

Certain Embodiments Include Methods Associated with Hormone Ablation Therapy

DACH1 regulates Estrogen and androgen signaling. Estrogen and androgen activity is targeted in therapies of diverse human diseases, e.g., cancer, including breast and prostate cancer, bone disease including osteoporosis, and other diseases. DACH1 repressed estrogen signaling (Popov V M, Cancer Res. (2009) Cancer Research 69:5752-60) and androgen signal transduction in prostate cancer (Wu K, et al., Cancer Research 2009 69(14):5752-60). DACH1 regulated the response of the androgen receptor to androgen antagonists (ie flutamide). Hormone receptor detection is routine for therapeutics selection, such as hormone ablation therapy on estrogen positive breast cancer and androgen positive prostate cancer. DACH1 may be a regulator of androgen therapy resistance. Certain embodiments include selecting treatment for disorders such as estrogen positive breast cancer and androgen positive prostate cancer by determining expression levels of DACH1. Treatments may include radiation, chemotherapy or hormone therapy.

### Certain Embodiments Include Use of DACH1 to Regulate Cell Stem Populations

Pluripotent stem cells have potential for organ regeneration, transplantation and for tissue repair. There are several key transcriptional factors in governing stem self-renewal and expansion, e.g., Sox2, Oct4, Nanog and KLF4. As described herein, DACH1 regulates such stem cell factors and their target genes expression. Regulation of DACH1 expression may be used to regulate stem cells expansion or contraction for patient therapy. Certain embodiments include use of DACH1 to regulate cell stem populations

### Certain Embodiments Include Selecting a Therapy to Treat a Disorder

Certain embodiments include selecting treatment for a subject with a disorder. In some such embodiments, the disorder can include cancer, e.g., breast cancer, lung cancer, kidney cancer, prostate cancer, etc. In some embodiments, the subject is human.

Some such embodiments include determining an expression level of a nucleic acid encoding DACH1 or DACH 1 protein in a sample obtained from a subject in need of treatment for cancer and/or additional markers such as an expression level of one or more nucleic acids encoding a gene selected from the group consisting of Sox2, Klf4, and Nanog or one or more proteins selected from the group consisting of SOX2, KLF4, and NANOG in the sample. Methods to determine expression levels of nucleic acids or proteins in a cell are well known in the art. Examples of such methods are described herein and include measuring the level of DACH1 mRNA in a cell of said sample, and determining an expression level of DACH1 protein in said sample comprises an immunoblot analysis.

Some embodiments include comparing the expression levels of certain nucleic acids and/or proteins in a sample with the expression levels of certain nucleic acids and/or proteins in normal tissue or a tissue with known metastatic potential. Some such embodiments include comparing the expression level of the nucleic acid encoding DACH1 or DACH1 protein and the expression level of the one or more nucleic acids encoding a gene selected from the group consisting of Sox2, Klf4, and Nanog or the one or more proteins selected from the group consisting of SOX2, KLF4, and NANOG in the sample to an expression level of a nucleic acid encoding DACH1 or DACH1 protein and an expression level of one or more nucleic acids encoding a gene selected from the group consisting of Sox2, Klf4, and Nanog or one or more proteins selected from the group consisting of SOX2, KLF4, and NANOG in normal tissue or cancerous tissue with a known metastatic potential.

More embodiments include selecting a treatment for the subject based on the determined expression level. For example, treatment can be selected for a subject with a decreased expression level of a nucleic acid encoding DACH1 or DACH1 protein in the sample. In some embodiments, the decreased expression level can comprise a decrease of at least about 5%, about 10%, about 20%, about 30%, about 40%, about 50%, about 60%, about 70%, about 80%, about 90%, and about 100%. In more embodiments, treatment can be selected for a subject with an increased expression level of the nucleic acid encoding one or more nucleic acids encoding a gene selected from the group consisting of Sox2, Klf4, and Nanog or the one or more proteins selected from the group consisting of SOX2, KLF4, and NANOG in the sample. In some embodiments, the increased expression level can comprise an increase of at least about at least about 5%, about 10%, about 20%, about 30%, about 40%, about 50%, about 60%, about 70%, about 80%, about 90%, and about 100%. In some embodiments, the increased expression level can comprise an increase of at least about at least about 2-fold, about 5-fold, about 10-fold.

In some embodiments, a treatment is selected in response to a determination of a reduced expression level of a nucleic acid encoding DACH1 or DACH1 protein in a sample. For example, a reduced expression level comprising at least about 50% can determine a selection for an aggressive treatment protocol. In some embodiments, a greater reduction in expression level can determine a selection of a more aggressive treatment protocol.

In some embodiments, a treatment is selected in response to an increased expression level of the nucleic acid encoding one or more nucleic acids encoding a gene selected from the group consisting of Sox2, Klf4, and Nanog or the one or more proteins selected from the group consisting of SOX2, KLF4, and NANOG in the sample. For example, an increased expression level comprising at least about 5-fold can determine a selection for an aggressive treatment protocol. In some embodiments, a greater increase in expression level can determine a selection of a more aggressive treatment protocol.

In some embodiments, the treatment can include administering a DNA-methyltransferase inhibitor to the subject, and administering to the subject an anti-IL8 therapy. In some embodiments, a treatment can include surgery, radiation therapy, proton therapy, chemotherapy, cryosurgery, and high intensity focused ultrasound.

### Certain Embodiments Include Inhibiting Expression of DACH1

Certain embodiments include reducing and/or inhibiting expression levels of DACH1 in a cell. In some embodiments, reduction of DACH1 expression can increase the stem cell-like phenotype of a cell. In some embodiments, reducing and/or inhibiting expression levels of DACH1 in a cell can increase proliferation of a cell, such as a stem cell. Examples of stem cells include neural stem cells, hematopoietic stem cells, muscle stem cells, and germ cells. Such cell populations can be useful in certain therapies e.g., tissue regeneration, such as neural tissue, muscle tissue, germ cells, and hematopoietic cells, e.g., in the treatment of cancers, e.g., leukemias.

Methods to reduce expression of a gene such as DACH1 are well known in the art. Some such methods are described herein. For example, some methods include contacting a cell with a nucleic acid encoding a portion of an antisense DACH1 gene. As used herein, “a portion of” refers to at least about 5%, 10%, 15%, 20%, 25%, 30%, 35%, 40%, 45%, 50%, 55%, 60%, 65%, 70%, 75%, 80%, 85%, 90%, 95%, and 100%.

An example of a human DACH1 mRNA is shown below (Accession number: NM_{—}080760; Version NM_{—}080760.4; GI259490229):

| | |
|---|---|
| . . . 1 | atctttgatc aatgtacttg ccagggagag cccaagtcct tcaaacctcc tccttttcac |
| 61 | cttcatcctt aactttgtgc tagagcgaga cccacacaac aacagccgac cctccccgcc |
| 121 | ccacccccac ccccaaacca gccctcgatc ccagcccccg gagaggactc gcatttcgac |
| 181 | ttgcgggaca cttttgtgcg ttcctctcca gagcgcctct cgtgctcgcc cctcttgcgc |
| 241 | tcgctcttta ttaccttcac ctccttttct cccccttctc tccctttctc cttctcgttc |
| 301 | tctcccggag ttgttgttgc ccccctcgct ccttctcccc ccttttttcc ccttcccctc |
| 361 | ccgggggtgt gtggcaactt ttcctctcgc ttctcctccg tctgtttccc cttatatgtg |
| 421 | accatggcag tgccggcggc tttgatccct ccgacccagc tggtcccccc tcaaccccca |
| 481 | atctccacgt ctgcttcctc ctctggcacc accacctcca cctcttcggc gacttcgtct |
| 541 | ccggctcctt ccatcggacc cccggcgtcc tctgggccaa ctctgttccg cccggagccc |
| 601 | atcgcttcgg cggcggcggc ggcggccaca gtcacctcta ccggcggcgg cggcggcggc |
| 661 | ggcggcggcg gcagcggagg cggcggcggc agcagcggca acggaggcgg cggtggcggc |
| 721 | ggcggcggtg gcagcaactg caaccccaac ctggcggccg cgagcaacgg cagcggcggc |
| 781 | ggcggcggcg gcatcagcgc tggcggcggc gtcgcttcca gcacccccat caacgccagc |
| 841 | accggcagca gcagcagcag cagtagcagc agcagcagca gcagcagtag tagcagcagc |
| 901 | agcagtagca gcagcagctg cggccccctc cccgggaaac ccgtgtactc aaccccgtcc |
| 961 | ccagtggaaa acacccctca gaataatgag tgcaaaatgg tggatctgag gggggccaaa |
| 1021 | gtggcttcct tcacggtgga gggctgcgag ctgatctgcc tgccccaggc tttcgacctg |
| 1081 | ttcctgaagc acttggtggg gggcttgcat acggtctaca ccaagctgaa gcggctggag |
| 1141 | atcacgccgg tggtgtgcaa tgtggaacaa gttcgcatcc tgaggggact gggcgccatc |
| 1201 | cagccaggag tgaaccgctg caaactcatc tccaggaagg acttcgagac cctctacaat |
| 1261 | gactgcacca acgcaagttc tagacctgga aggcctccta agaggactca aagtgtcacc |
| 1321 | tccccagaga actctcacat catgccgcat tctgtccctg gtctcatgtc tcctgggata |
| 1381 | attccaccaa caggtctgac agcagccgct gcagcagctg ctgctgctac caatgcagct |
| 1441 | attgctgaag caatgaaggt gaaaaaaatc aaattagaag ccatgagcaa ctatcatgcc |
| 1501 | agtaataacc aacatggagc agactctgaa aacggggaca tgaattcaag tgtcgatgag |
| 1561 | accccgcttt ctacaccaac cgcaagagac agccttgaca aactctctct aactgggcat |
| 1621 | ggacaaccac tgcctccagg ttttccatct ccttttctgt ttcctgatgg actgtcttcc |
| 1681 | atcgagactc ttctgactaa catacagggg ctgttgaaag ttgccataga taatgccaga |
| 1741 | gctcaagaga aacaggtcca actggaaaaa actgagctga agatggattt tttaagggaa |
| 1801 | agagaactaa gggaaacact tgagaagcag ttggctatgg aacaaaagaa tagagccata |
| 1861 | gttcaaaaga ggctaaagaa ggagaagaag gcaaagagaa aattgcagga agcacttgag |
| 1921 | tttgagacga aacggcgtga acaagcagaa cagacgctaa aacaggcagc ttcaacagat |
| 1981 | agtctcaggg tcttaaatga ctctctgacc ccagagatag aggctgaccg cagtggcggc |
| 2041 | agaacagatg ctgaaaggac aatacaagat ggaagactgt atttgaaaac tactgtcatg |
| 2101 | tactgaatct ttcctgttga agaaatccat gttatagaaa agaactttgc agtcagacat |
| 2161 | tcgtcatggg aaagttcaga aaaaaataaa gtccttttaa gggaacttcc tgaattttgt |
| 2221 | gtattaatgt tctttaaaag tttaagtatt ctacaaaaaa aaaaaaagtt ttctccattg |
| 2281 | attttcacct gtggttcata ccagagacct gagaatgttt gtaaatgtac aagtatcaaa |
| 2341 | gttcttacag ttaattactg caacttgctg ctggacaatt gtatacagag ttaaaggcag |
| 2401 | gtctgaataa gacctagctt tgtttttttc taatggaatg aaccattttc ctcttctgaa |
| 2461 | aattctgtat ctgagcacat caagagactc ttgtagcagt ggttacccag acttacagaa |
| 2521 | ttatgtcctc cagaaaccag caagaacact tggaatgaac gaatgaactt gtagggggca |
| 2581 | tagaggattc ttgaaaaaaa aaaatgcaag agtgattttc tgttacattc aatttcaaac |
| 2641 | tctctaattg tgggttttct cctgaagaat tttttttcac atactttcca aaagaccaac |
| 2701 | aaatggatgt tgacaacaac ccaatgaaat aacattttgc atatctgaaa agaagcattg |
| 2761 | aatataagcc aaaagctttc actgaaggtt tttttttctt aaaaataaaa aaaaatatat |
| 2821 | aagtgtaaca tgttttcatt ccaaactggt agtggtatat agaattaaag ataataatgt |
| 2881 | tgcttcttat tcaaactgtt ggtcatatgt acagtatata aacataaaac acacaaggaa |
| 2941 | ggtattatgt atgcagtagt atactagagt ttaggaaaat gaaaatttta gaaaatatgt |
| 3001 | tttgtcaccc tgttggtcag aaagatgtct ttctggtttt aacgcatgca ggcatgtaaa |
| 3061 | tatttgtctg gagtcacagt attaatgaat gagatcttaa gcatctggtg acatcagaac |
| 3121 | tctgtgtcag ccacttttat ttgtatattg aaccctagct agtgccccaa gctgcactat |
| 3181 | tgggaatgga ttgtggctga acagcaaatc aaaacaccag aaatattttt atatgttaac |
| 3241 | gtcatattat gttaatgttg ctgaaaacaa aacctaacaa accttgatgt accagtccaa |
| 3301 | taccatgtag cgctgagtga taaagttaaa atgtgctgtg cttcccaccc ttgtcagagg |
| 3361 | gaagggtggc tatgtgttat tttcactgtc tttttgaaag ttacagtatg tgttttcact |
| 3421 | ttcgtgcaga taactggaag taaagcggca aacagtgctt attacatgct aaagttacct |
| 3481 | tctctttgtt ttttgcatat ctggaattac acctttaaag actgatatga atcagtacgg |
| 3541 | tcactataca ttttatgatt tttctgtcat cttaaaattg tatgatcgta acattattta |
| 3601 | ttaccacaaa acagcaaaat cttcaatgtc taagaaaact agcttaaaat gtttaaatat |
| 3661 | agttctgatt gggtattaat tacttgatta agaaaaaatt aacattatag atactctggc |
| 3721 | attacgcttc tatacctttt aggtcttcct tgcaatactg gaacataatt cttttgtgta |
| 3781 | gctcactatt agccagctaa gttcatcttt ttaataccat aaaaaggtta tatgtacagt |
| 3841 | tcctatttta gcttgcttac aaagggagca ttatttttat ttaaagtatt gctagtaaat |
| 3901 | gatttgtaga aacttggttt tctaagcata gttcttccat aaccaccttt tgttgtttga |
| 3961 | gcacaaggga ttcttttcct agttctatgt gtttgtttcc ctatatgcag tctttaaagg |
| 4021 | attacaacac ttaaaattga atggacttgt gtcaagcttt ttgcatcata cattttttga |
| 4081 | aagattttta aaaaagccta caacttacat atgtagtaga atcagccatt gctctgctcc |
| 4141 | tggcatagag tcacctgttt gttatgtgga ttaaatagtt ttaaaataca tatttgaaga |
| 4201 | cctttgagaa tgctttagtg tttgatttga aataaaagga aattttagca aggattaaag |
| 4261 | aaaaaagcta tcagctgtat gttaagagag actcttacta acatgttgta aatattacaa |
| 4321 | ttcatgaaat gttattgtaa gtctgtaact taattttttc cctgttttag ttatacaggt |
| 4381 | tggtttggaa atttgtgttt tggcataaac aagtaaaatg tgcccatttt atggtttcca |
| 4441 | tgcttttgta atcctaaaaa tattaatgtc tagttgttct atattataac cacatttgcg |
| 4501 | ctctatgcaa gcccttggaa cagaacatac tcatcttcat gtaggaccta tgaaaattgt |
| 4561 | ctatttttat ctatatattt aaagttttct aaaaatgata aaaggttatt acgaattttg |
| 4621 | ttgtacaaaa tctgtacaaa aatctgtttt tacatcataa tgcaagaatt ggaaattttt |
| 4681 | ctatggtagc ctagttattt gagcctggtt tcaatgtgag aaccacgttt actgttattg |
| 4741 | tatttaattt tcttttcctt ttcaacaatc tcctaataaa actgtctgaa atctcaaaaa |
| 4801 | aa |

### EXAMPLES

### Mammosphere Formation and FACS Analysis of Stem Cell Surface Markers

Mammosphere formation assays were conducted as described in Lindsay J, et al. “ErbB2 induces Notch1 activity and function in breast cancer cells.” Clinical and Translational Science 2008; 1(2): 107-15. Immunostaining of cell surface markers by FACS analysis for breast cancer stem cells was based on Shackleton M, et al. “Generation of a functional mammary gland from a single stem cell.” Nature 2006; 439: 84-8. Before labeling, the cells were blocked with normal mouse IgG in 1/100 dilution for 30 min and then incubated with PE labeled mouse anti-human CD24 (⅕) (clone ML5, BD Pharmingen, San Diego, Calif.) and/or PE/Cy5 labeled rat anti human/mouse CD44 ( 1/200) (clone IM7, BioLegend, San Diego, Calif.) for 1 hour. All experiments were conducted at 4° C. Cell sorting was performed on a FACSCalibur cell sorter (BD Bioscience, San Jose, Calif.). The data were analyzed with FlowJo software (Tree Star, Inc., Ashland, Oreg.).

### Cell Culture, Plasmid Construction, Reporter Genes, Expression Vectors, DNA Transfection, and Luciferase Assays

Cell culture, DNA transfection, and luciferase assays using the Sox-2-Luc and Nanog-Luc reporter genes were performed as described in Fu et al., “Acetylation of the androgen receptor enhances coactivator binding and promotes prostate cancer cell growth.” Mol Cell Biol 2003; 23: 8563-75; Fu M, et al. Androgen receptor acetylation governs trans activation and MEKK1-induced apoptosis without affecting in vitro sumoylation and trans-repression function. Mol Cell Biol 2002; 22: 3373-88; and Fu M, et al. p300 and p300/cAMP-response element-binding protein-associated factor acetylate the androgen receptor at sites governing hormone-dependent transactivation. J Biol Chem 2000; 275: 20853-60. Expression vectors encoding KLF4/c-Myc and Oct4/Sox 2 were from Addegene. The Met-1 cells were cultured in DMEM supplemented with 10% fetal calf serum, 1% penicillin, and 1% streptomycin. The MCF10A and MCF10A-Myc lines were previously described (Wu, K. et al. (2006) Mol Cell Biol 26, 7116-7129. The expression plasmids encoding an N-terminal FLAG peptide linked to DACH1, DACH1 DS-domain alone (DS) or DACH1 DS-domain deleted (ΔDS) were previously described. Lentiviral DACH1 shRNA was from Open BioSystems. Transfection and infection were performed using standard protocols. GFP positive were selected by FACS. Cells were plated at a density of 1×10⁵ cells in a 24-well plate on the day prior to transfection with Superfect according to the manufacturer's protocol (Qiagen, Valencia, Calif.). A dose-response was determined in each experiment with 50 and 200 ng of expression vector and the promoter reporter plasmids (0.5 μg). Luciferase activity was normalized for transfection efficiency using B-galactosidase reporters as an internal control. The fold effect of expression vector was determined with comparison to the effect of the empty expression vector cassette and statistical analyses were performed using the t-test.

### RNA Isolation, RT-PCR and Quantitative Real-Time PCR

Total RNA was isolated from Met-1 cells infected with the DACH1 expression vector system, using Trizol (Sakamaki T, et al. Cyclin Dl determines mitochondrial function in vivo. Mol Cell Biol 2006; 26: 5449-69). SYBR Green based real-time PCR reactions were performed using QuantiTect SYBR Green PCR kit (Qiagen Inc, Valencia, Calif.) and Quantitect pre-validated Primer Assays for mouse and 18S rRNA as internal control following manufacturers recommendations on an ABI Prism 7900HT system (Applied Biosystems Inc., Foster City, Calif.). Oligonucleotides used for RT-PCR include those shown in Table 1. 18S rRNA oligos were used as control (Sakamaki T, et al. Cyclin Dl determines mitochondrial function in vivo. Mol Cell Biol 2006; 26: 5449-69).

TABLE 1

| SEQ ID NO | Primer | Sequence |
|---|---|---|
| SEQ ID | Nanog Forward | CAGAAAAACCAGTGGTTGAAGACTAG |
| NO: 01 | | |
| SEQ ID | Nanog Reverse | GCAATGGATGCTGGGATACTC |
| NO: 02 | | |
| SEQ ID | Oct4 Forward: | CTGTAGGGAGGGCTTCGGGCACTT |
| NO: 03 | | |
| SEQ ID | Oct4 Reverse | CTGAGGGCCAGGCAGGAGCACGAG |
| NO: 04 | | |
| SEQ ID | Sox2 Forward: | GGCAGCTACAGCATGATGCAGGAGC |
| NO: 05 | | |
| SEQ ID | Sox2 Reverse | CTGGTCATGGAGTTGTACTGCAGG |
| NO: 06 | | |
| SEQ ID | KLF4 Forward: | TGCCAGACCAGATGCAGTCAC |
| NO: 07 | | |
| SEQ ID | KLF4 Reverse | GTAGTGCCTGGTCAGTTCATC |
| NO: 08 | | |
| SEQ ID | c-Myc Forward: | TGAGCCCCTAGTGCTGCAT |
| NO: 09 | | |
| SEQ ID | c-Myc Reverse | AGCCCGACTCCGACCTCTT |
| NO: 10 | | |

Oligonucleotides for chromatin immune-precipitation (ChIP) were directed to murine SOX2.

Distant site:
Forward Primer (SEQ ID NO: 11):
5′-GCAGTGAGAGGGGTGGACTA-3′;
Reverse Primer (SEQ ID NO: 12):
5′-CTCCCCTCATCTACCCCAAC-3′.
Proximal site (sox2 binding site):
Forward Primer (SEQ ID NO: 13):
5′-CGCAGAAACAATGGCACACCAC-3′;
Reverse Primer (SEQ ID NO: 14):
5′-CCGTTTTCAGCAACAGGTCACG-3′.
Nanog Distant site:
Forward Primer (SEQ ID NO: 15):
5′-GGCAAACTTTGAACTTGGGATGTGGAAATA-3′;
Reverse Primer(SEQ ID NO: 16):
5′-CTCAGCCGTCTAAGCAATGGAAGAAGAAAT-3′.
Proximal site (oct4-sox2 binding site):
Forward Primer (SEQ ID NO: 17):
5′-GGATGTCTTTAGATCAGAGGATGCCC-3′;
Reverse Primer (SEQ ID NO: 18):
5′-CCACAGAAAGAGCAAGACACCAACC-3′.

### Microarray and Cluster Analysis

DNA-free total RNA isolated from Met-1 cells expressing GCP or DACH1 were used to probe Affymetrix Gene 1.0 arrays (Affymetrix, Santa Clara, Calif.). RNA quality was determined by gel electrophoresis. Probe synthesis and hybridization were performed as previously described (Li Z, et al. Alternate Cyclin D1 mRNA Splicing Modulates p27KIP1 Binding and Cell Migration. J Biol Chem 2008; 283: 7007-15). Analysis of the arrays was performed using the Gene Spring. Arrays were normalized using robust multiarray analysis (RMA), and P-value of 0.05 was applied as statistical criteria for differential expressed genes. These genes were then grouped using hierarchical clustering with “complete” agglomeration, and each cluster was further analyzed based upon the known function of the genes contained in the cluster. Expression profiles are displayed using Treeview (Eisen M B, et al. Cluster analysis and display of genome-wide expression patterns. Proc Natl Acad Sci USA 1998; 95: 14863-8). Classification and clustering for pathway level analysis using gene sets associated employed Analysis of Sample Set Enrichment Scores (ASSESS) available at http://people.genome.duke.edul assess (Edelman, et al. Analysis of sample set enrichment scores: assaying the enrichment of sets of genes for individual samples in genome-wide expression profiles. Bioinformatics 2006; 22: e 1 08-16).

ASSESS provides a measure of enrichment of each gene set in each sample. Gene set enrichment was dependent on a concordance of at least two samples within the replicates that was opposite between phenotypes.

### Immunohistochemistry, Chromatin Immune-Precipitation and ChIP-seq Analysis

Immunohistochemical analysis of human breast cancer was conducted using a polyclonal DACH1 antibody (Wu, K., et al. (2006) Mol Cell Biol 26, 7116-7129). Human breast cancer tissue arrays were from Biomax, US. Chromatin immune precipitation assays were conducted as previously described using antibodies directed to the Flag epitope of DACH1 protein. ChIP-seq was conducted as previously described (Hulit, J., et al. (2004) Mol Cell Biol 24, 7598-7611; and Wu, K., et al. (2007) Mol Biol Cell 18, 755-767).

### Migration and Invasion Assays

The inverse invasion assay was performed as described in Malliri A, et al. “The transcription factor AP-1 is required for EGF-induced activation of rho-like GTPases, cytoskeletal rearrangements, motility, and in vitro invasion of A431 cells.” J Cell Biol 1998; 143: 1087-99). Met-1 cells transfected with GFP control or DACH1 were allowed to attach to the underside (bottom) of the growth factor-depleted Matrigel-coated polycarbonate chambers (Transwell 8 μm pore size filters). The cells were then chemoattracted (10% FCS) across the filter and through the Matrigel above. Cells were fixed in 4% paraformaldehyde and GFP fluorescence was analyzed in z-sections (1 section every 4 μm) from the bottom of the filter using a confocal microscope (Bio-Rad MRC 1024). Three-dimensional reconstructions of the GFP-expressing cells into the Matrigel and then pixel quantification were done using the Volocity computer software (Improvision). Percentage of invading cells is the ratio of pixels above the filter (into Matrigel) to the total number of pixels above and below the filter. Met-1 cells were sorted CD24^{high} or CD24^{low} and seeded on an 8 μm pore size Transwell filter insert (Costar) coated with thin layer of matrigel for migration assay as previously described (Wu K, et al. Dachshund inhibits oncogene-induced breast cancer cellular migration and invasion through suppression of interleukin-8. Proc Natl Acad Sci USA 2008; 105: 6924-9).

### Nude Mice Study

1×10⁵ Met-1 cells expressing GFP control or DACH1 were implanted subcutaneously into 4- to 6-week-old athymic female nude mice purchased from NCI. The tumor growth was measured twice weekly by digital caliper for 7 weeks. Tumor weight was measured when mice were sacrificed on day 35 after cells implantation.

### Results

### DACH1 Expression is Reduced in Breast Cancer Cell Lines Enriched for Cancer Stem Cells

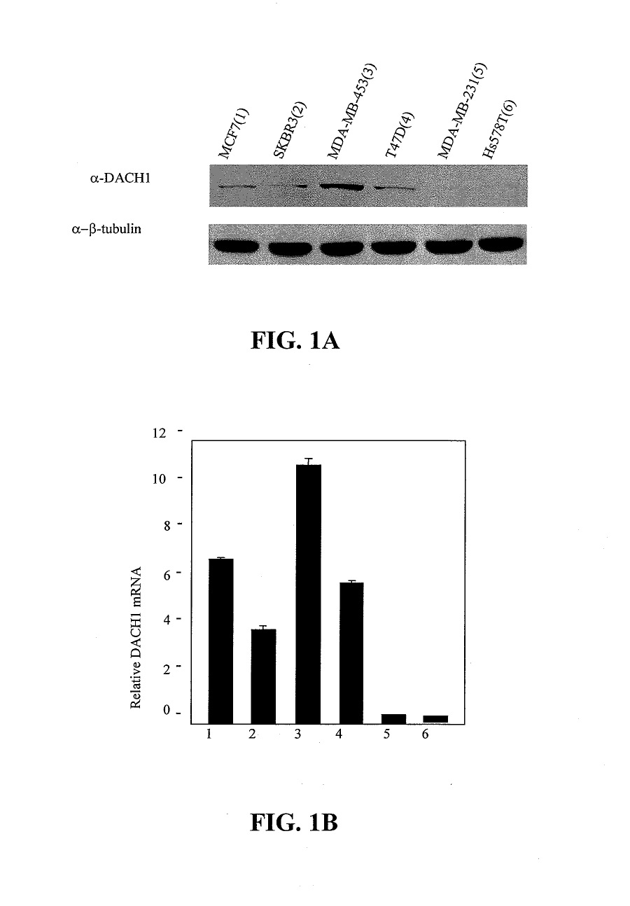

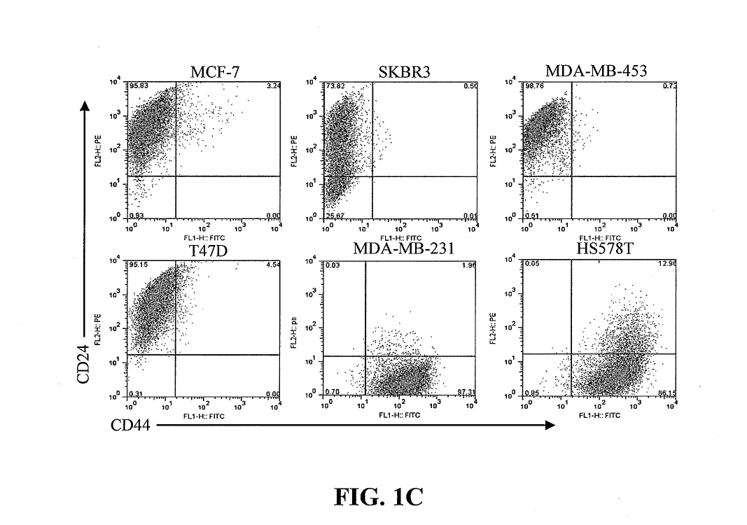

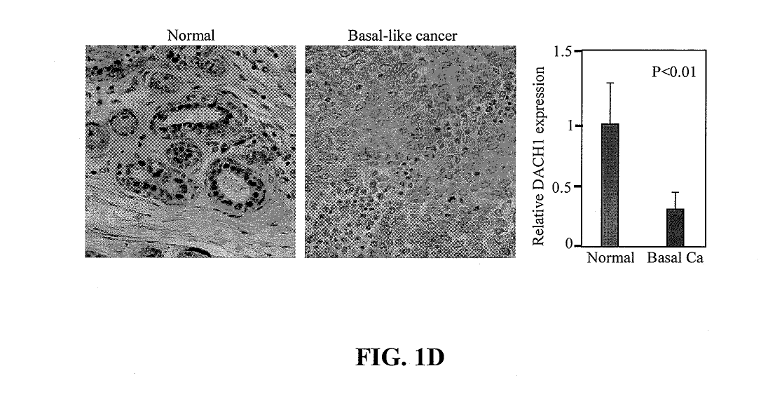

Loss of DACH1 expression correlates with poor prognosis in human breast cancer and DACH1 inhibits MCF7 cell proliferation in tissue culture. In order to characterize further the expression of DACH1 in breast cancer cell types. Western blot analysis was conducted using a previously characterized polyclonal antibody (Wu, K., et al. (2006) Mol Cell Biol 26, 7116-7129) Quantitation of relative abundance from multiple experiments demonstrated a reduction of DACH1 abundance in the MDA-MB231 and HS578T cells (FIG. 1A; FIG. 1B). Immunoepitope staining for the breast cancer stem cell markers CD44⁺/CD24⁻demonstrated a relative increase in the proportion of CD44⁺/CD24⁻ cells in the MDA-MB231 and HS578T cells (FIG. 1C). Distinct subtypes of human breast cancer include the basal-like, luminal (A and B), Her2⁺ and normal breast like carcinomas with distinct prognostic significance. Basal-like breast carcinomas are of high grade with a distinctive proclivity to metastasize and express genes associated with the maintenance of the stem cell phenotype (Ben-Porath, I., et al. (2008) Nat Genet 40, 499-507). Comparison between normal human breast epithelial cells, and basal-like vs non basal-like showed a significant reduction in mRNA expression and in DACH1 abundance in the basal-like tumors (FIG. 1D).

### DACH 1 Expression Inhibits the Proportion of Breast Cancer Cells Expressing Cancer Stem Cell Markers In Vivo

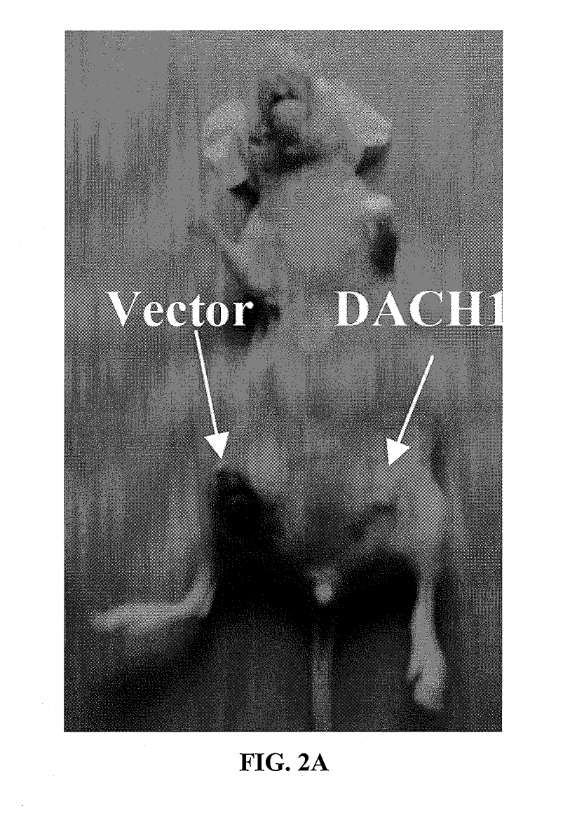

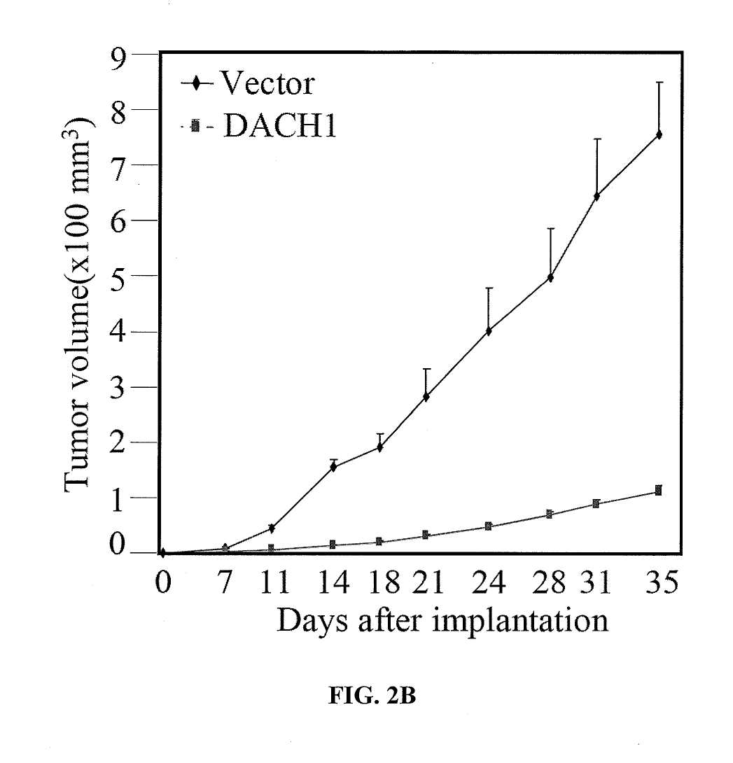

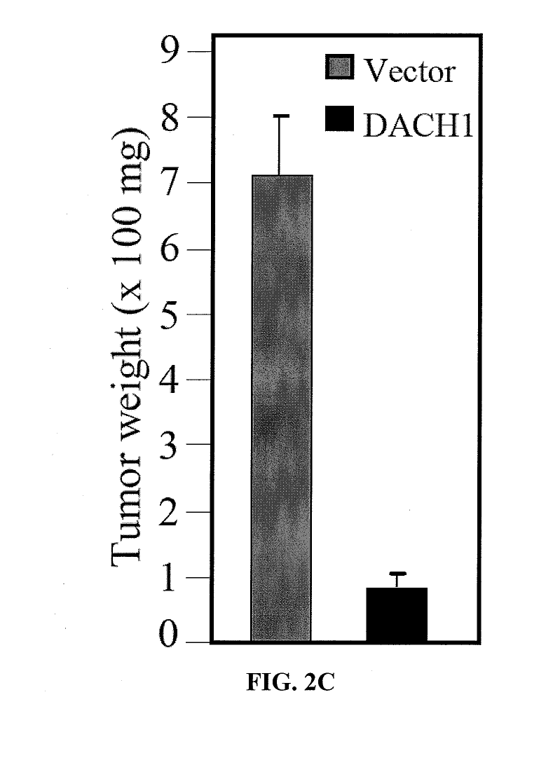

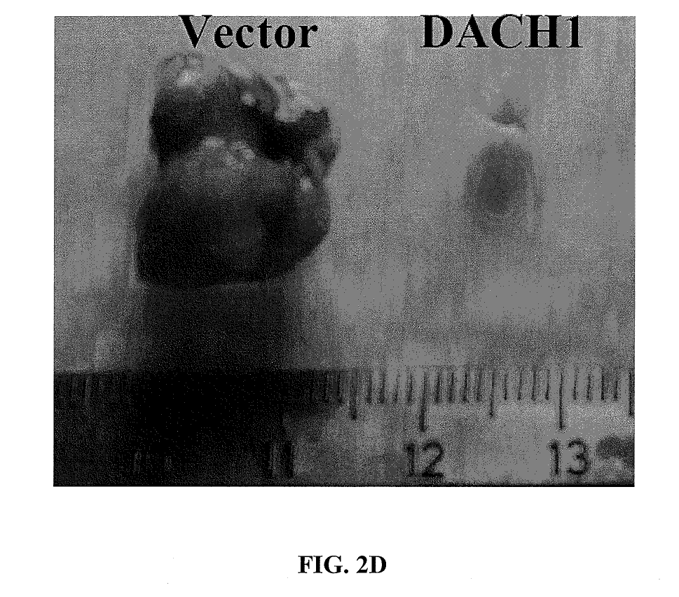

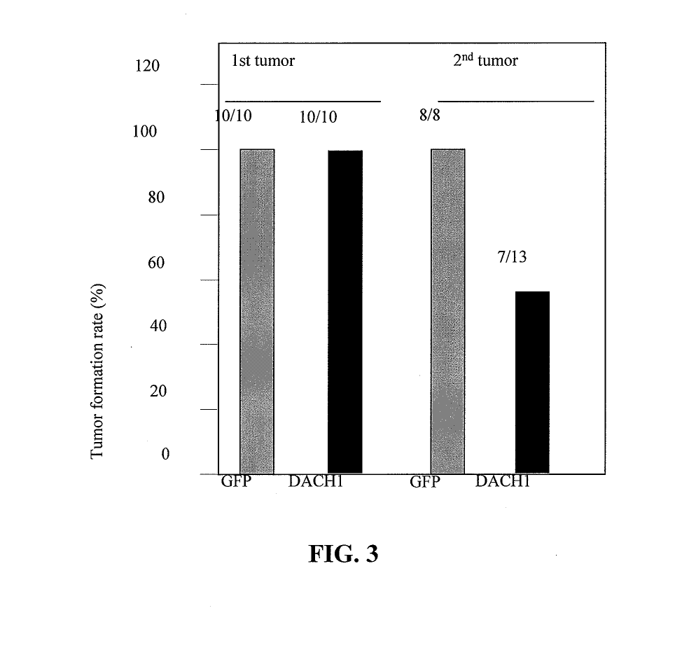

Highly metastatic breast cancer cell line Met-1 was transduced with an expression vector encoding DACH1 or a control vector. MET-1 cells were transduced with a DACH1 expression vector resulting in a ˜2-fold increase in DACH1 expression by Western blot analysis (data not shown). Immunohistochemistry demonstrated the presence of the DACH1-tagged Flag epitope throughout the cell population. The effect of DACH1 on mammary tumor growth in vivo was assessed by implantation in nude mice (FIG. 2A). DACH1 expression reduced the volume of tumors by ˜80%. Tumor weight was reduced by ˜90% (FIG. 2B). FIG. 2D shows a photograph of a tumor transfected with DACH1 and a control tumor transfected with vector only. Serial transplantation experiments demonstrated a ˜50% reduction in new tumor formation of DACH1 expressing Met 1 breast cancer cells (FIG. 3).

### DACH1 Inhibits Mammosphere Formation and the CD44⁺/CD24⁻ Phenotypes

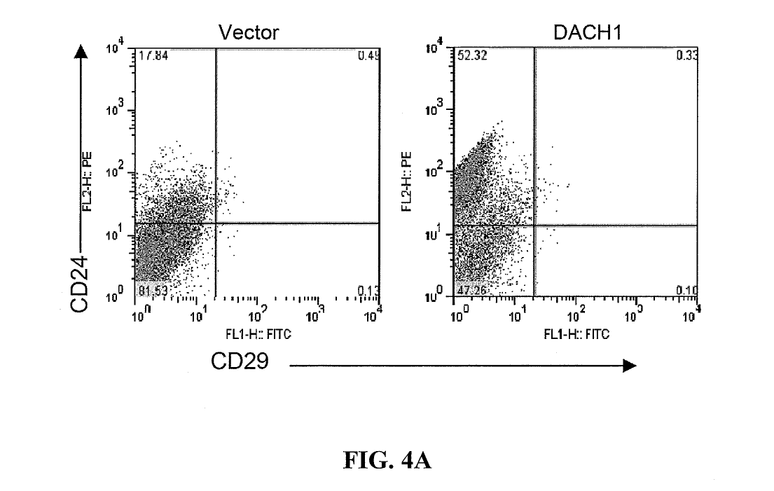

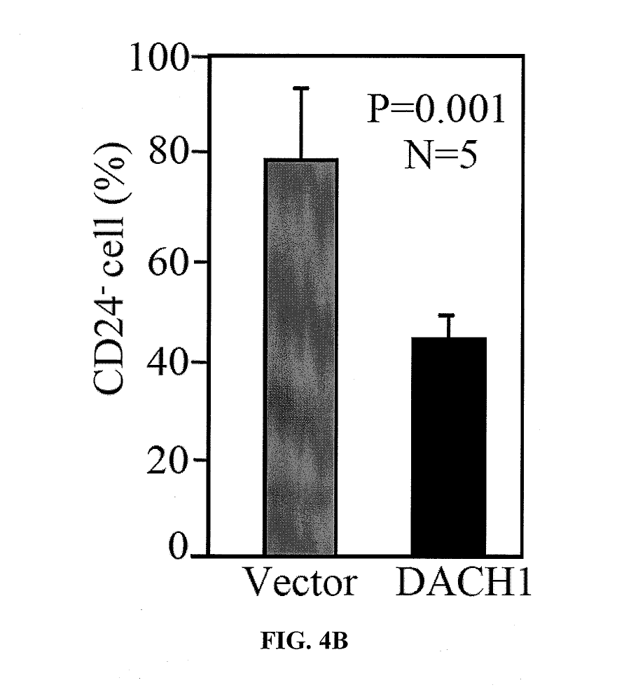

Cancer stem cells can be enriched by sorting for CD24^{-/low} cells. In order to determine whether DACH1 expression regulated the relative proportion of CD24^{-low} breast tumor cells in vivo Met-1 cells transduced with either a DACH1 expression vector or a control vector were implanted into nude mice. Tumors were grown for 3 weeks in mice and subsequently analyzed for CD24^{-low} cells. Induction of DACH1 expression reduced the proportion of CD24^{-/low} cells by −50% (FIGS. 4A and 4B). A small number of primary breast cancer cells, tumor initiating cell (TIC) or cancer stem cells form secondary tumors (Boyer L A, et al. Core transcriptional regulatory circuitry in human embryonic stem cells. Cell 2005; 122: 947-56). TICs form non-adherent mammospheres when cultured under specific conditions in the absence of serum.

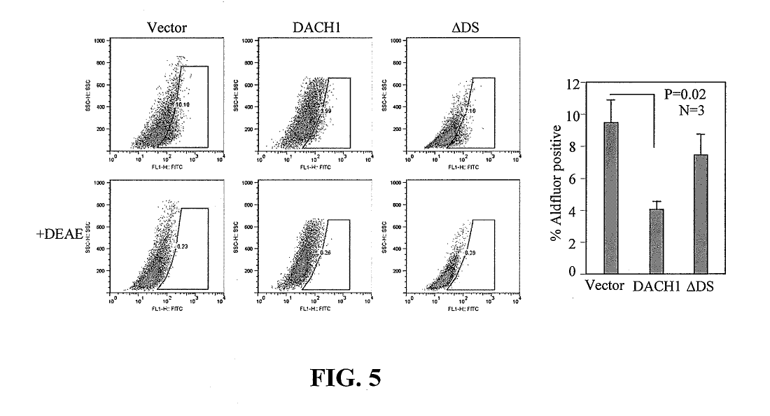

As a complementary assay of the BTIC phenotype Aldefluor staining was conducted as previously described (Charafe-Jauffret, E., et al. (2009) Cancer Res 69, 1302-1313). The stem cell marker aldehyde dehydrogenase (ALDH) is thought to regulate stem cell differentiation through metabolism of retinal to retinoic acid. The fluorescent aldefluor assay measures ALDH activity and has been used to isolate cancer stem cells from brain tumors, multiple myekma, acute myeloid leukemia and breast cancer. DACH1 expression reduced the proportion of Aldefluor-positive cells by ˜60% (FIG. 5). Expression of a DNA-binding defective mutant of DACH1 (ΔDS) was defective in reducing Aldefluor staining (FIG. 5).

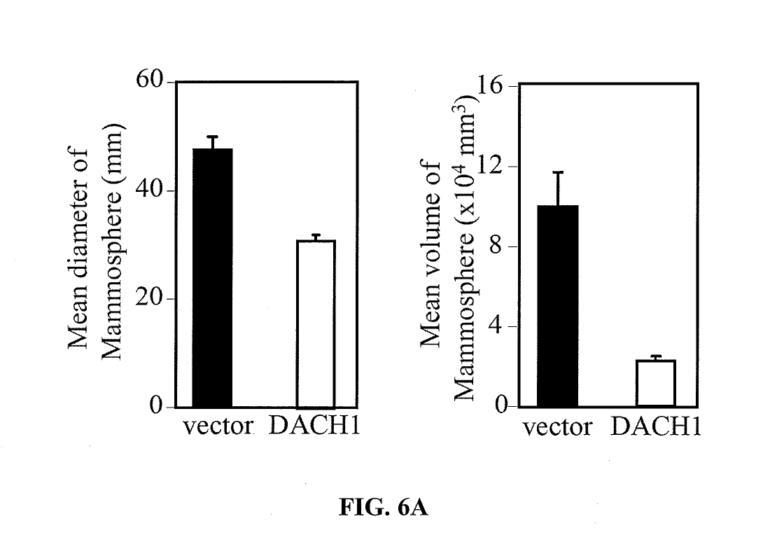

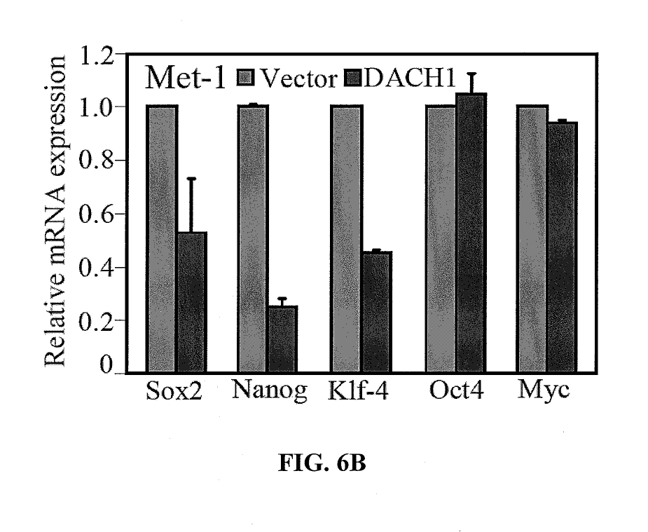

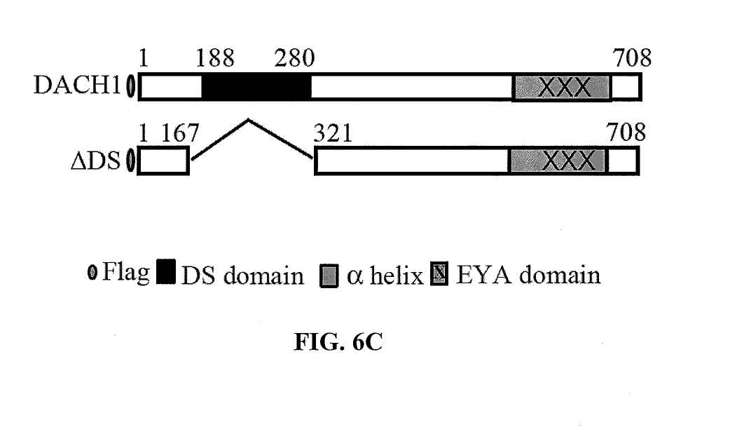

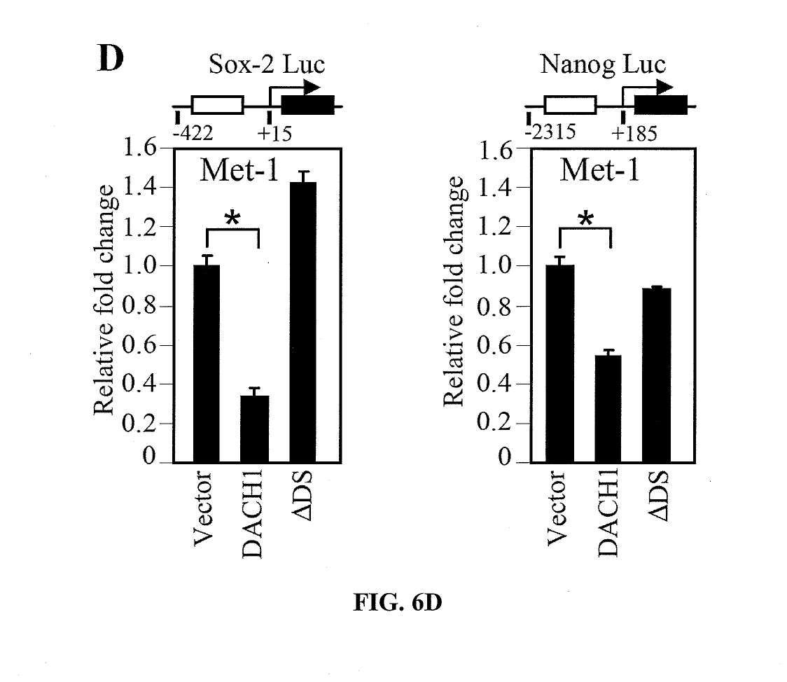

The cancer stem cell hypothesis suggests that many cancers are maintained in a hierarchal organization of cancer “stem cells” or tumor initiating cells rapidly dividing amplifying cells (early precursor cells) and differentiated tumor cells. Cancer stem cells are thought to contribute to tumor progression, therapy resistance and recurrence and can be enriched by cell sorting for CD44high/CD24−/low cells. A small number of primary breast cancer cells, tumor initiating cell (TIC) or cancer stem cells form secondary tumors. TICs form non-adherent mammospheres when cultured under specific conditions in the absence of serum. In order to examine further the role of DACH1 in TIC, mammosphere assays were conducted with the Met-1 mammary tumor cell lines. Induction of DACH1 reduced mammosphere number by >60% in cell lines (FIG. 6A).

Quantitation of the relative expression of the ES cell markers (Sox2, Oct4, Nanog, KLF and c-Myc) was conducted using mRNA from the MET-1 tumors expressing DACH1 or control (FIG. 6B). QT-PCR analysis demonstrated a reduction in the abundance of Sox2, Nanog and KLF4. Each of these genes promotes stem cell expansion. As DACH1 had reduced the expression of Sox2 and Nanog, studies were conducted to determine whether the Sox2 and Nanog genes were directly repressed by DACH1. DACH1 deletion constructs were created (FIG. 6C). The promoter of the Sox2 and the Nanog genes were directly repressed by DACH1 expression (FIG. 6D). Deletion of the DACH1 DS domain abrogated transcriptional repression DACH1 (FIG. 6D).

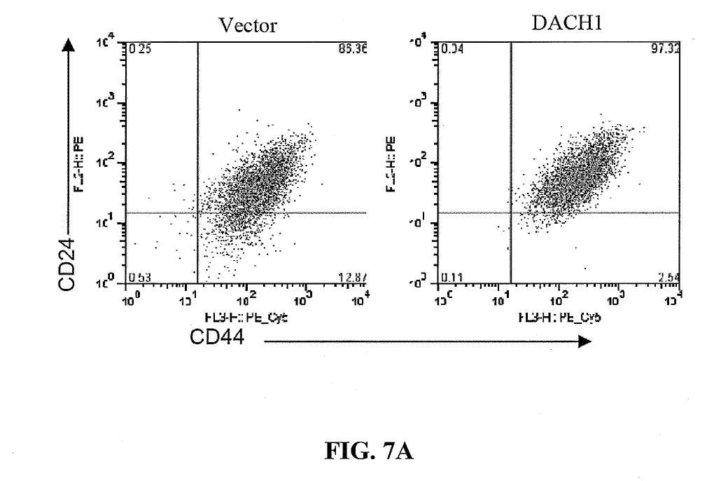

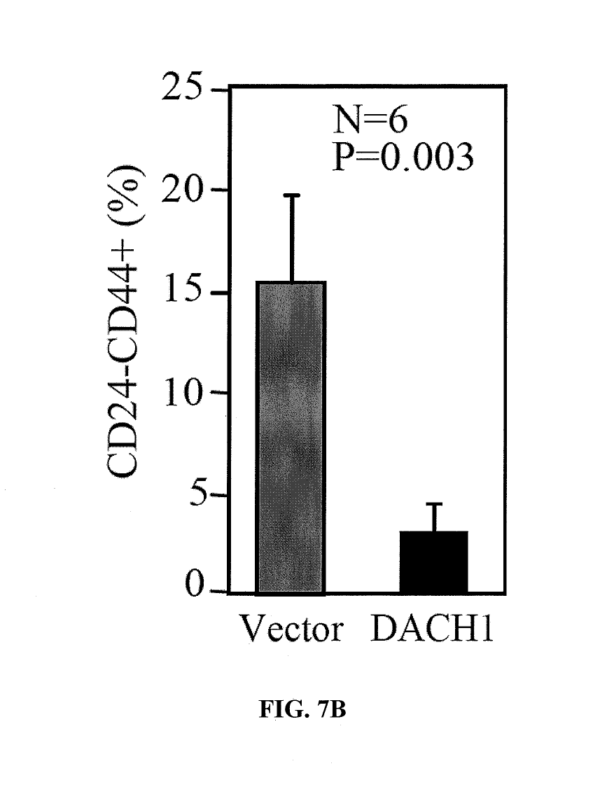

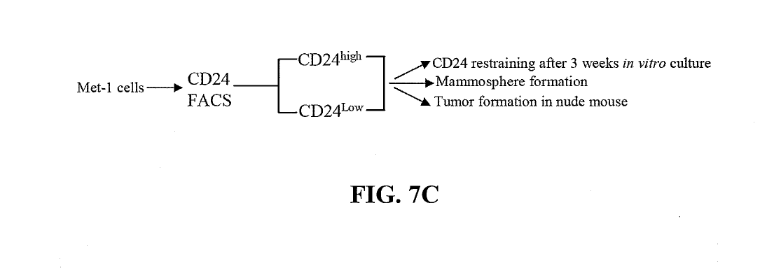

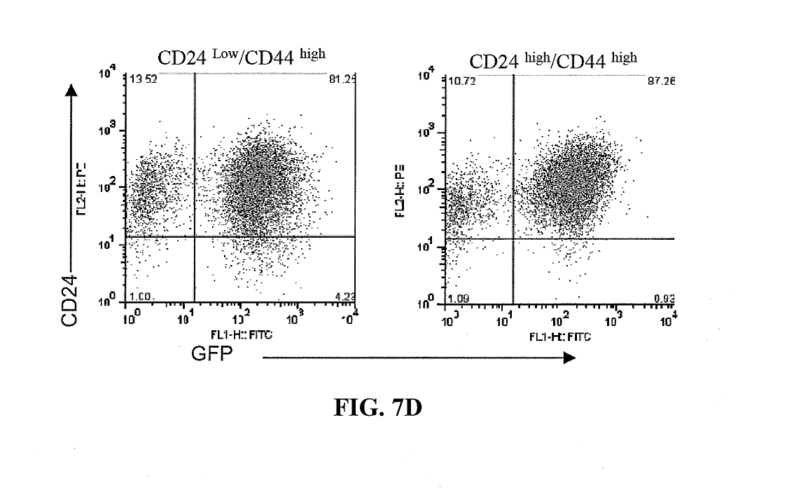

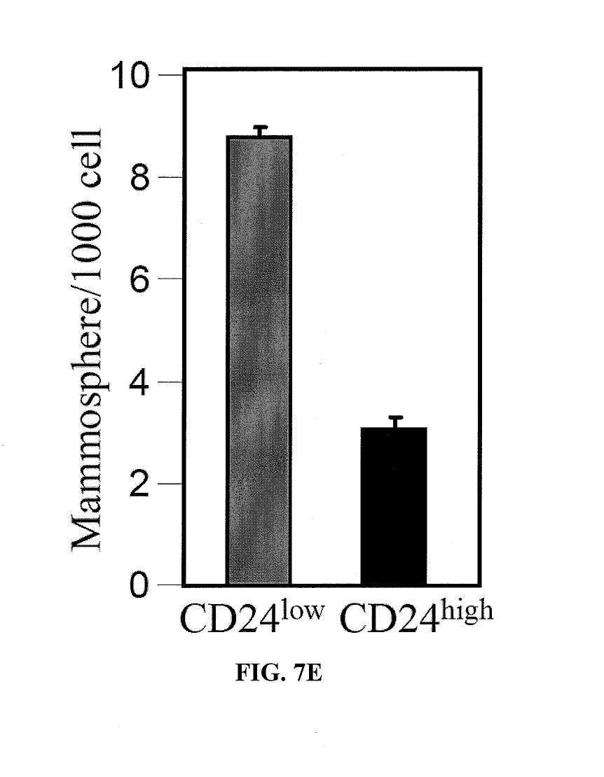

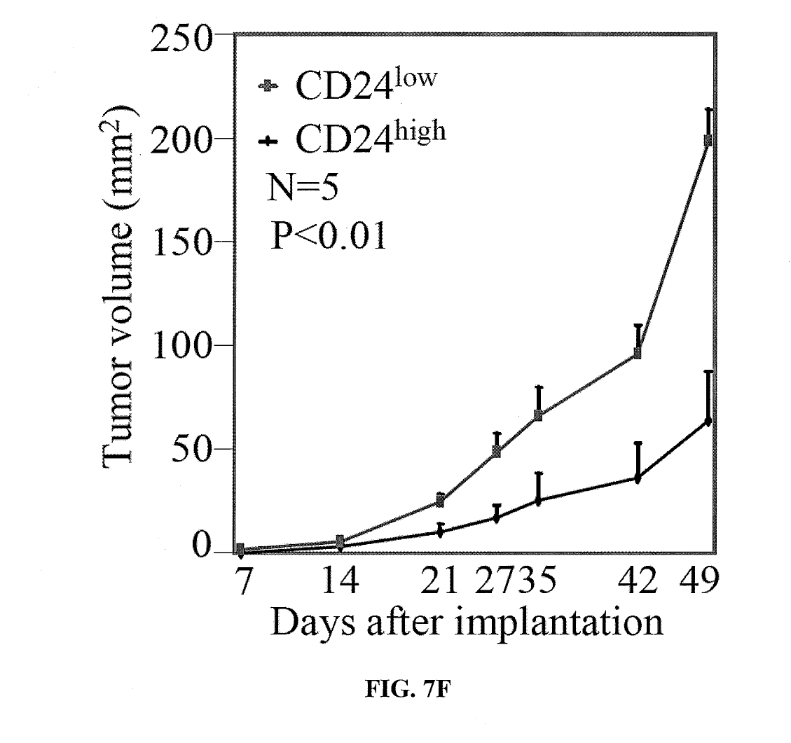

DACH1 expression in Met-1 cells reduced the relative proportion of CD44^{high}/CD24^{low} cells by ˜80% (FIG. 7A and FIG. 7B, 15% vs. 3%, n=6, P<0.003). In order to examine the biological significance of DACH1 mediated inhibition of the CD24 population, Met-1 cells were subjected to FACS analysis for the CD24^{high} vs. CD24^{low} populations. Multipotentiality of the CD24^{high} and CD24^{low} populations was determined by their ability to form CD24^{high} and CD24^{low} populations and to form mammospheres as a surrogate measure of stem cell expansion (FIG. 7C). CD24^{low}/CD44⁺ cells and CD24^{high}/CD44⁺ cells were separated by FACS analysis and grown in cultures for 3 weeks. Restaining by FACS demonstrated CD24^{low}/CD44^{high} gave rise to both CD24^{high}/CD44^{high} and CD24^{low}/CD44^{high} whereas CD24^{high}/CD44^{high} gave rise to only the parental CD24^{high}/CD44^{high} population (FIG. 7D). The CD24^{low} and CD24^{high} Met-1 cells were next examined for mammosphere formation. The CD24^{low} cells gave a 4-fold greater yield of mammospheres (FIG. 7E). These studies suggest the CD24^{low} and CD44^{high} cells maintain multipotentiality. To determine the tumor growth characteristics of these two distinct Met-1 cell populations, tumor implantation analysis was conducted. The CD24^{low}/CD44^{high} grew 4 times larger tumors that CD24^{high}/CD44^{high} Met-1 cells (FIG. 7F).

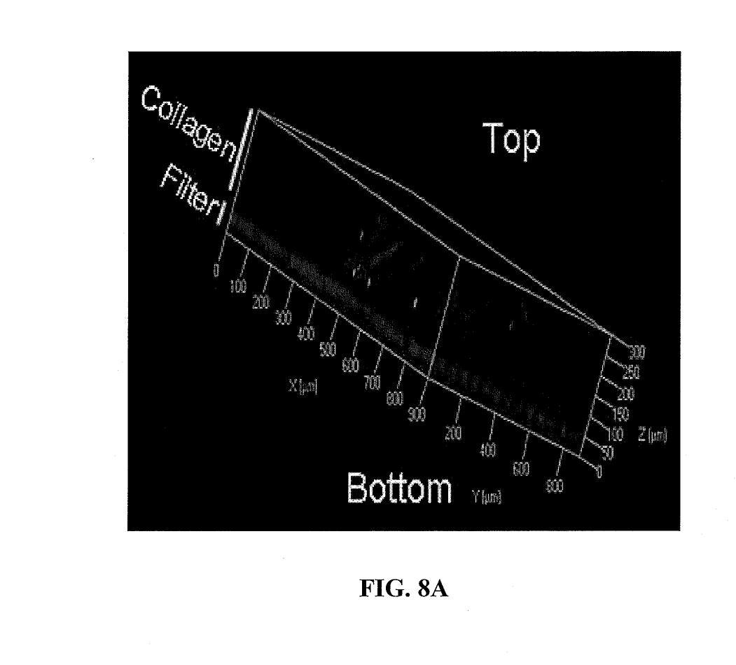

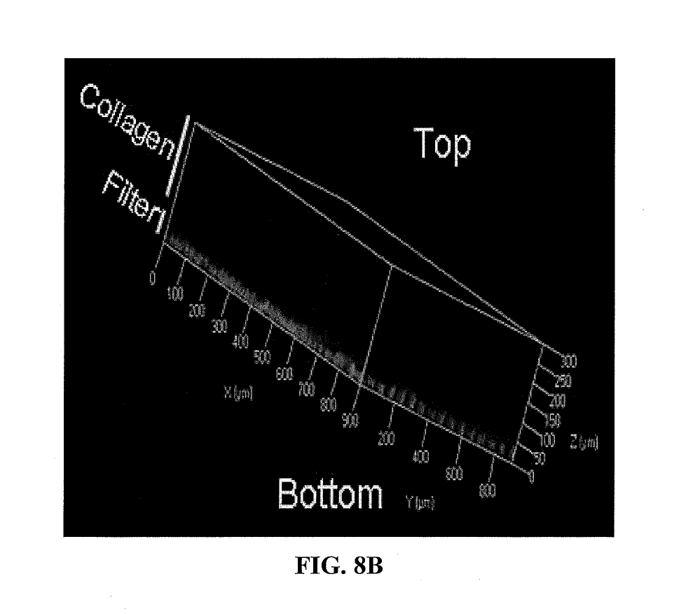

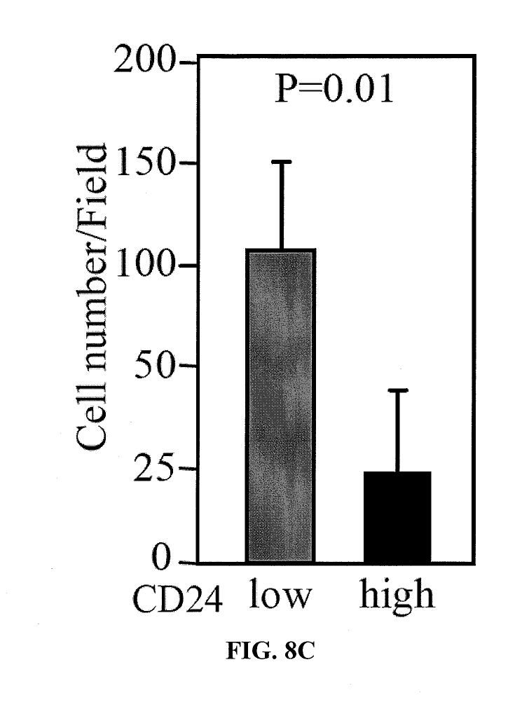

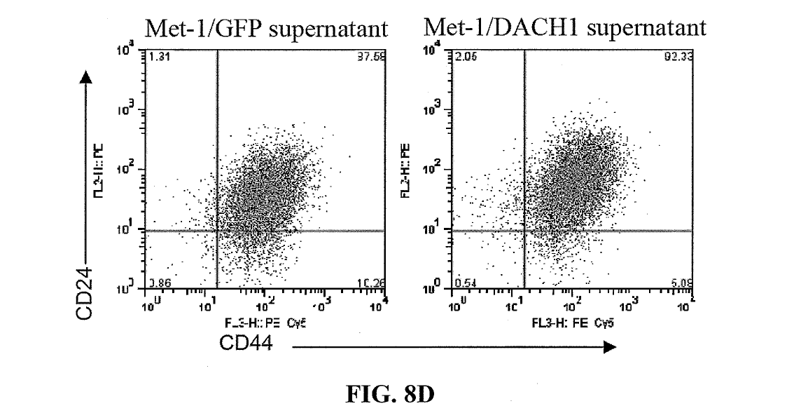

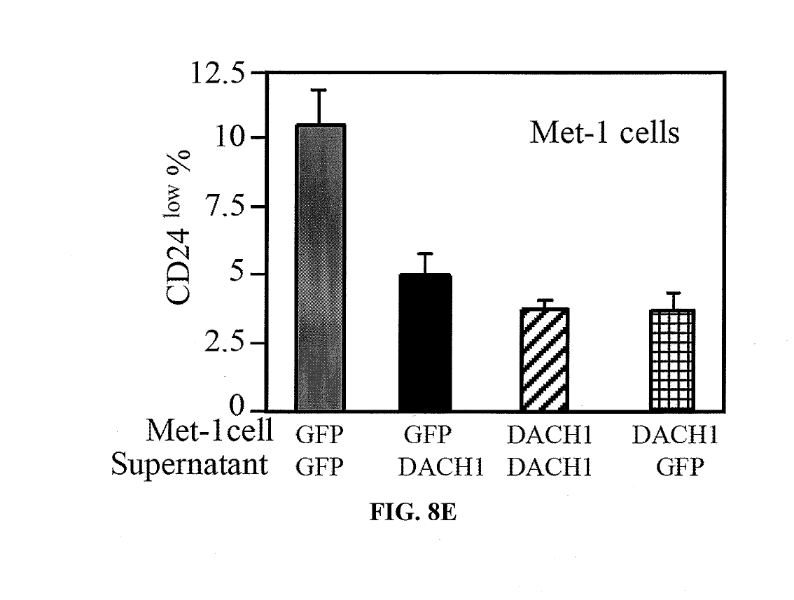

To examine further the regulation of invasiveness by DACH1, three-dimensional matrigel-matrix invasiveness assays were conducted (FIG. 8A and FIG. 8B). The proportion of invasive Met-1 cells was reduced ˜90% by DACH1 expression. CD24^{low}/CD44^{high} cells subpopulation of Met-1 cells demonstrated more migration than the CD24^{high}/CD44^{high} (FIG. 8C). In order to examine further the molecular mechanisms by which DACH1 regulates the proportion of CD44^{high}/CD24^{low} Met-1 cell we considered the possibility that DACH1 may regulate the production of a secreted factor. The conditioned medium of DACH1-transduced Met-1 cells was added to Met-1 cells, and FACS analysis was conducted after 7 days. The conditioned medium of DACH1 expressing cells was sufficient to reduce the proportion of CD44^{high}/CD24^{low} cells (FIG. 8D and FIG. 8E). The addition of DACH1 conditioned medium had no additional effect on the relative proportion of CD44^{high}/CD24^{low} cells in Met-1 cells expressing DACH1.

### Endogenous DACH1 Inhibits the Stem Cell Phenotype

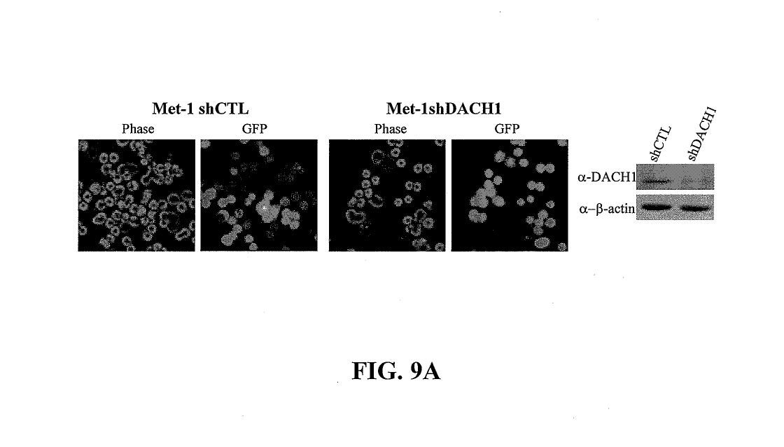

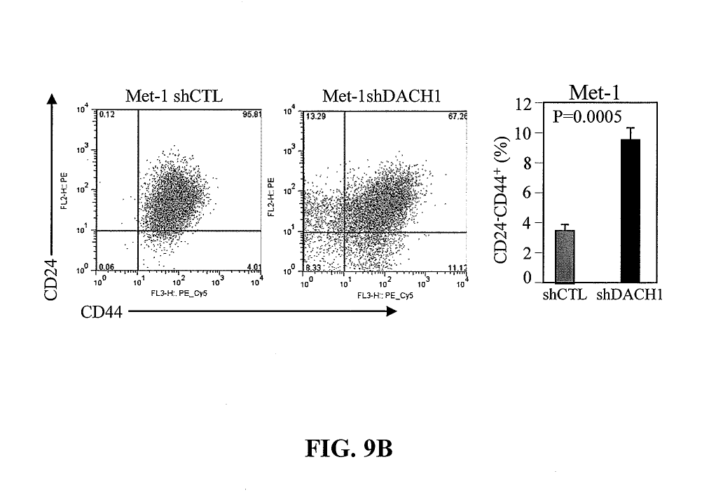

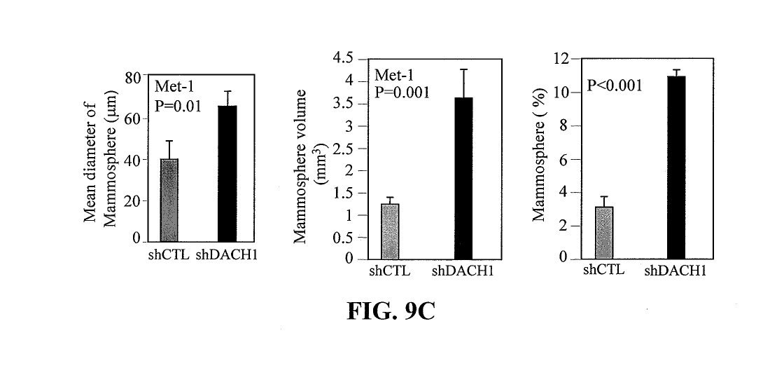

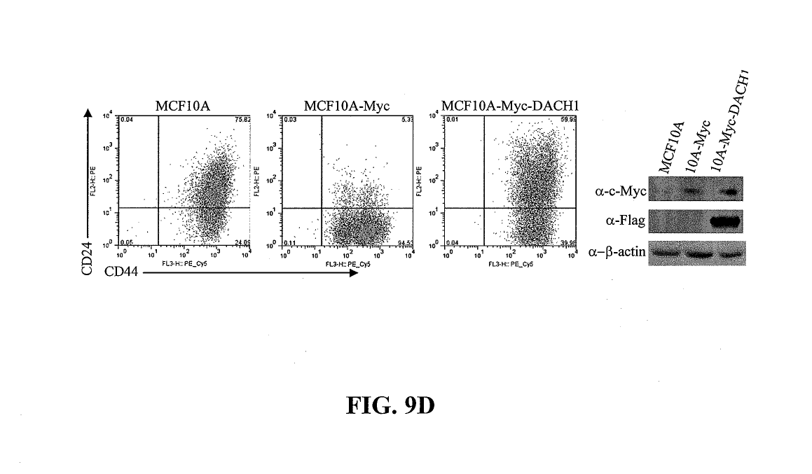

These studies suggested that a modest induction of DACH1 expression was sufficient to inhibit mammosphere formation and the relative proportion of cells with features of breast cancer stem cells. In order to determine whether endogenous DACH1 functioned to inhibit cancer stem cells a lentivirus encoding DACH1 shRNA linked via an IRES to GFP was used to transduce Met-1 cells (FIG. 9A). Comparison was made to the control vector. Reduction of DACH1 abundance with DACH1 shRNA in multiplicate experiments increased the proportion of CD44⁺/CD24⁻ cells ˜2.2-fold (FIG. 9B). The number of mammospheres reflects the relative proportion of progenitor cells, whereas the size of the mammosphere may also be affected in part by the proliferative capacity of the cells. Mammosphere volume was increased 3.5 fold by DACH1 ShRNA expression (FIG. 9C). The relative number of mammospheres was increased 350% by DACH1 ShRNA (FIG. 9C).

c-Myc transduction of the immortal human MCF10A cells induced cells with contact-independent growth properties; and increased the proportion of CD44⁺/CD24⁻ cells (FIG. 9D) from ˜24% to 95%. Transduction of MCF10-c-Myc cells with DACH1 inhibited the proportion of breast cancer stem cells from 95% to ˜40% (FIG. 9D). These findings suggest endogenous DACH1 is a key determinant of mammosphere number and therefore of BTIC.

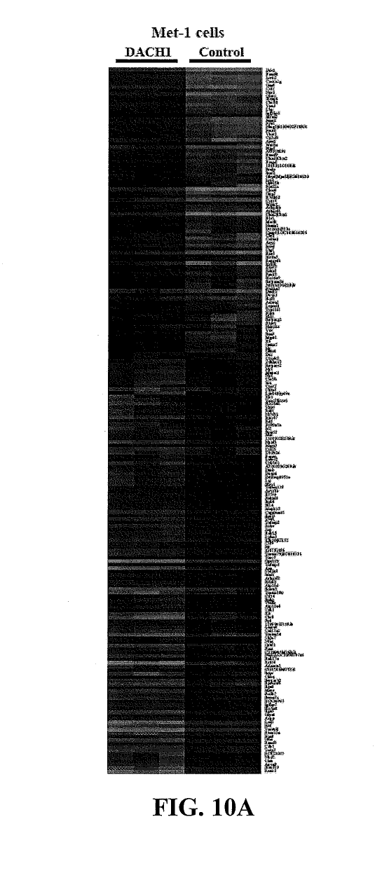

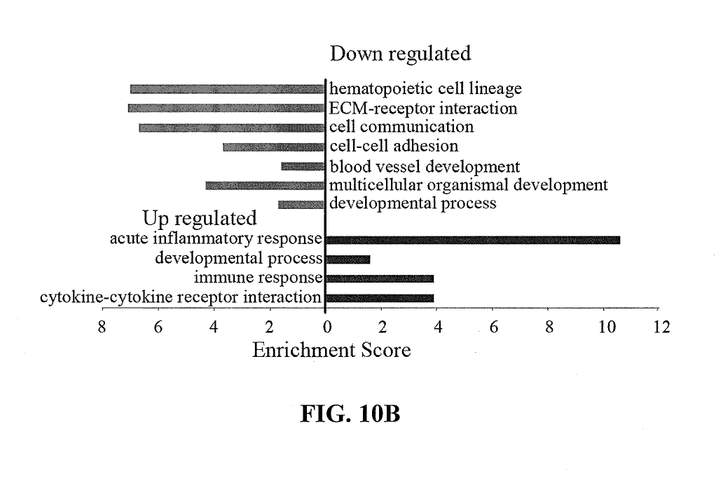

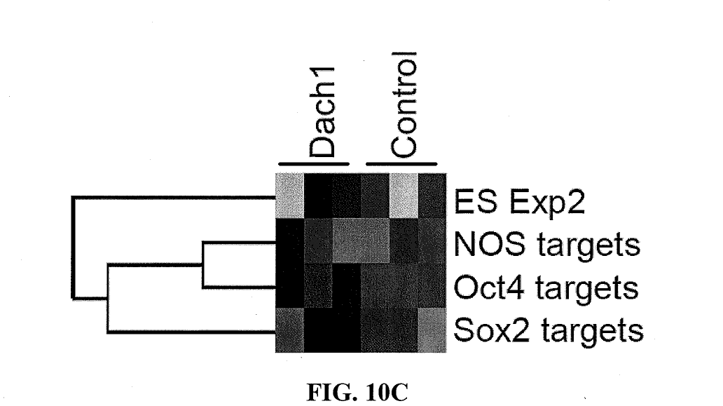

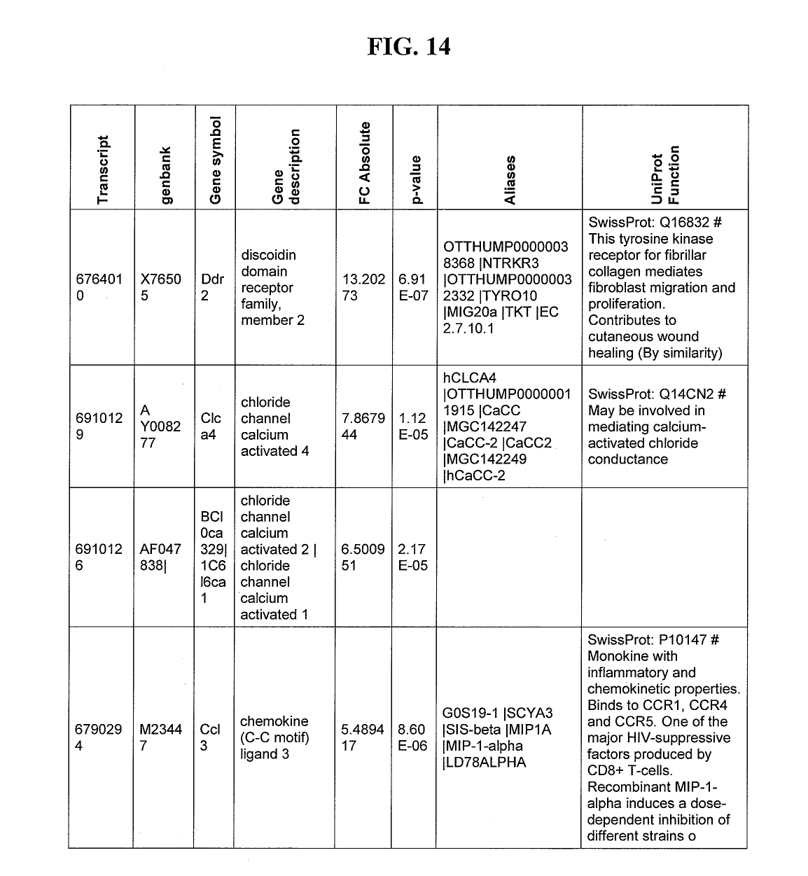

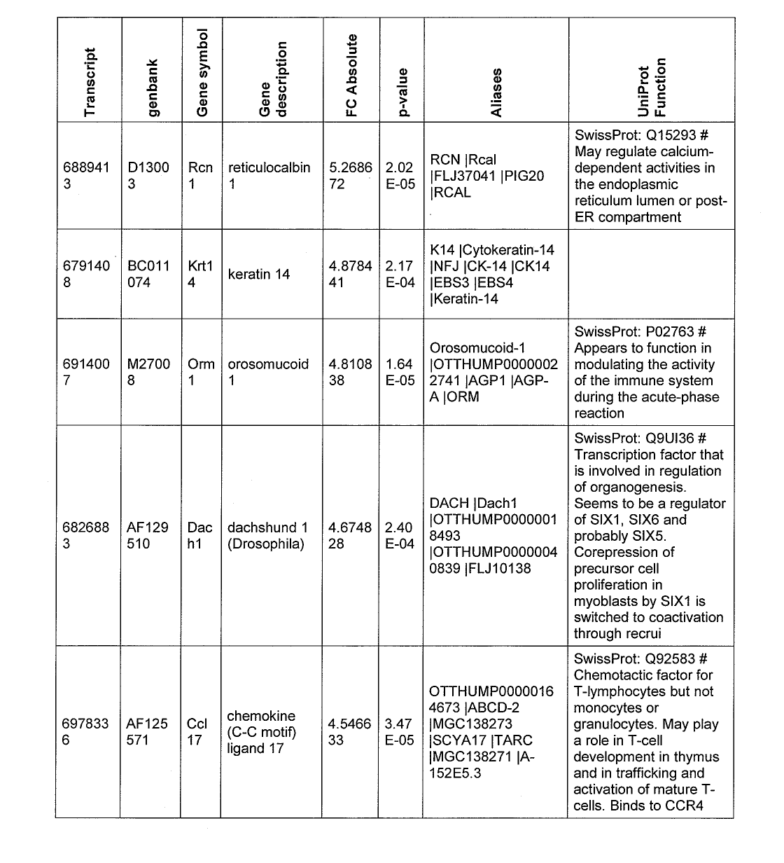

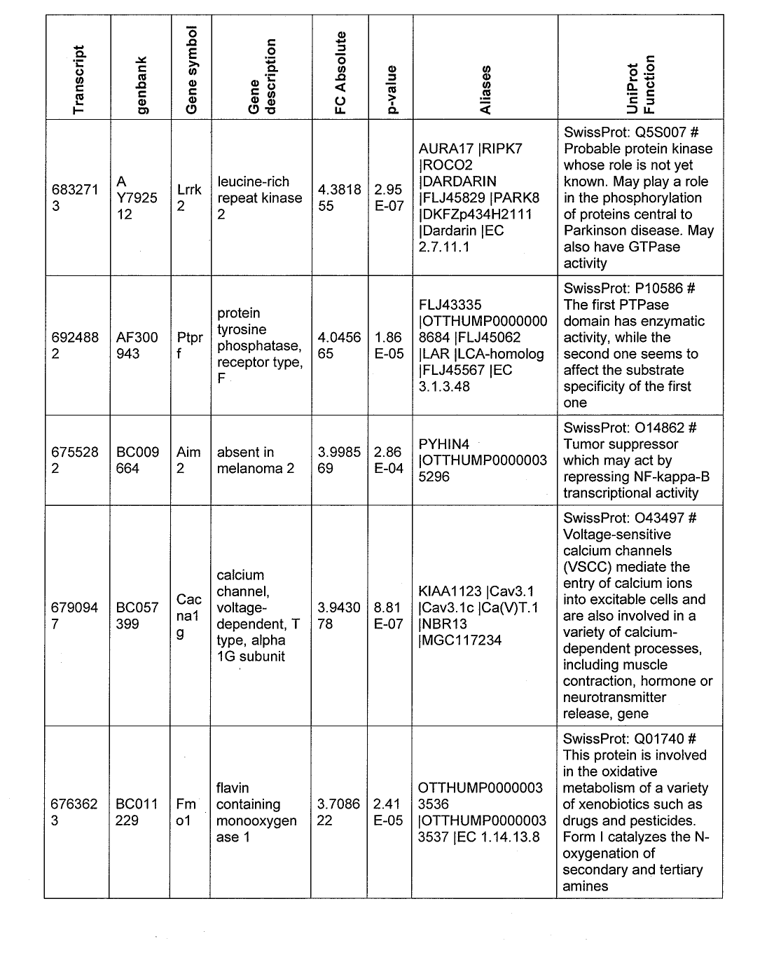

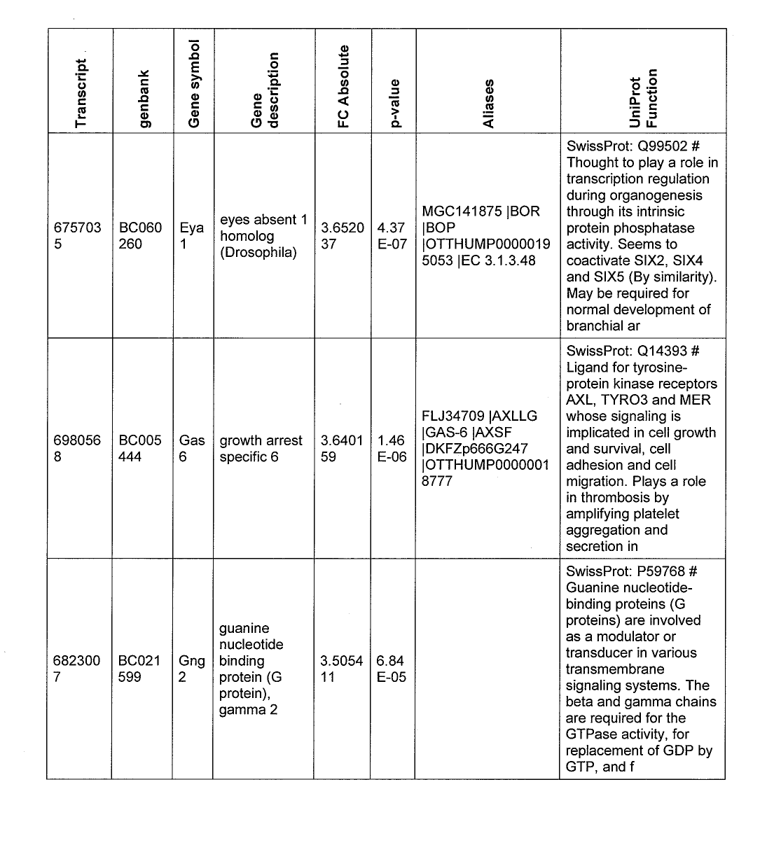

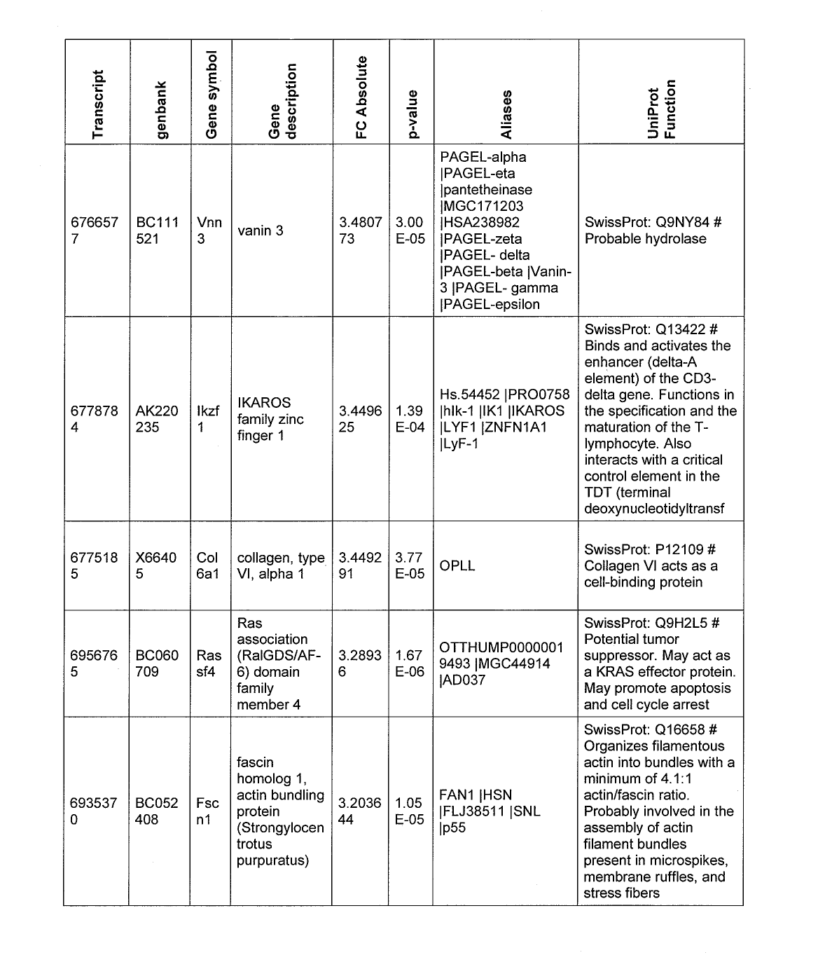

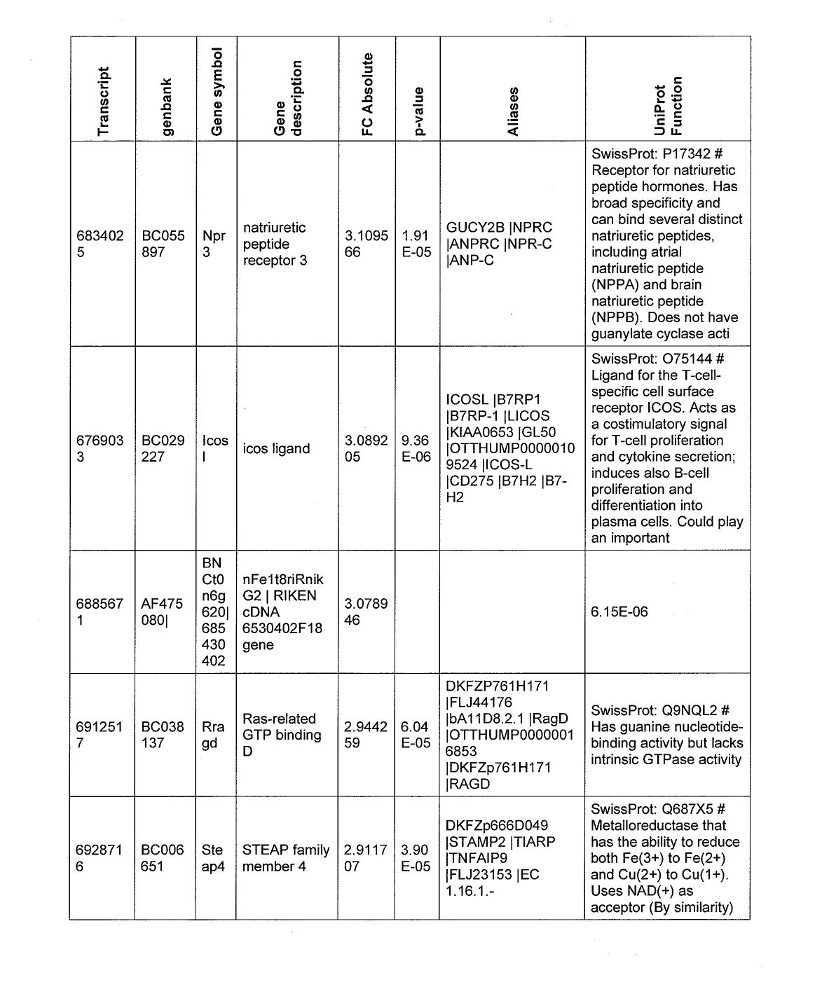

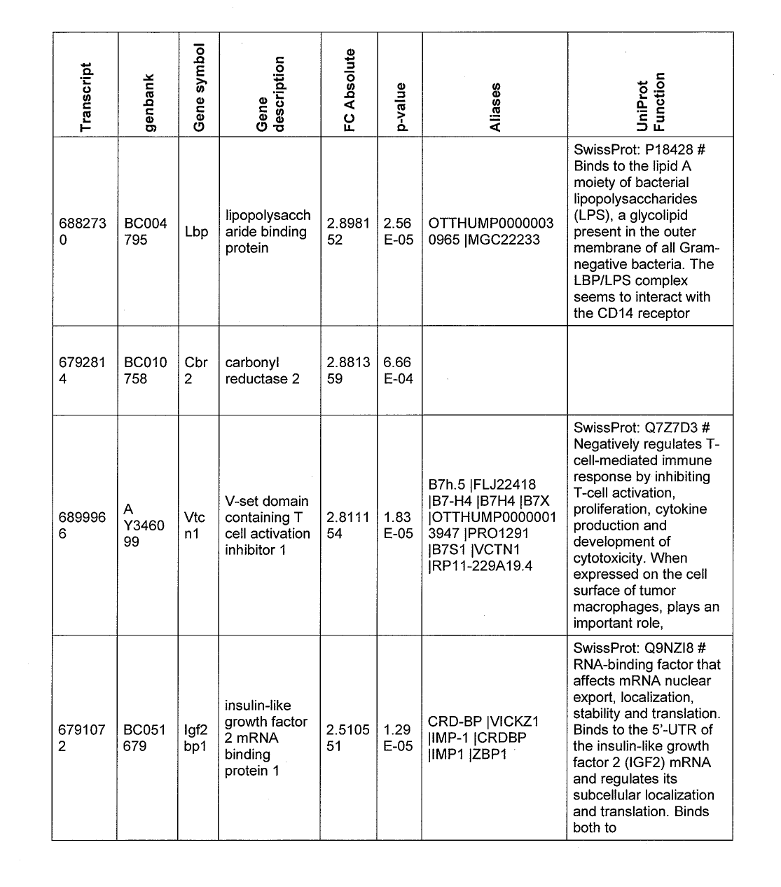

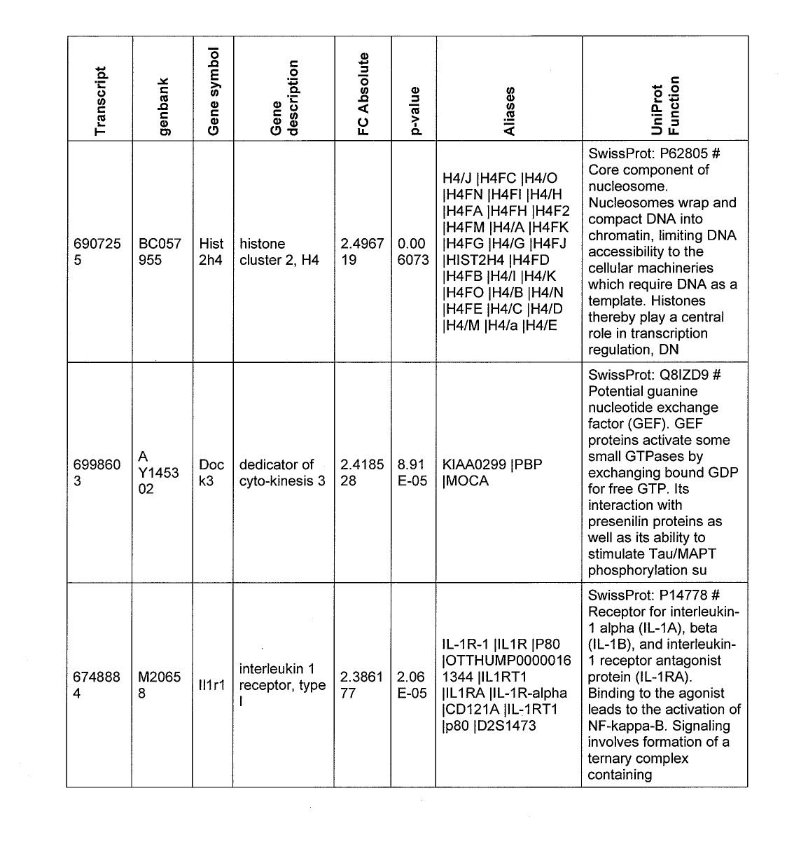

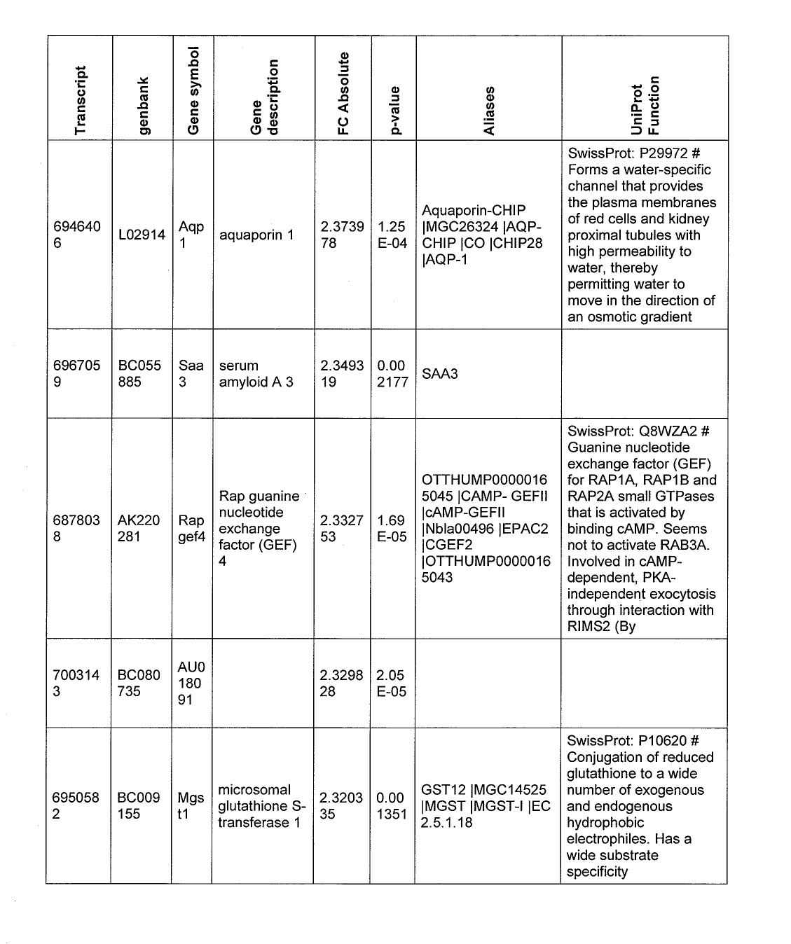

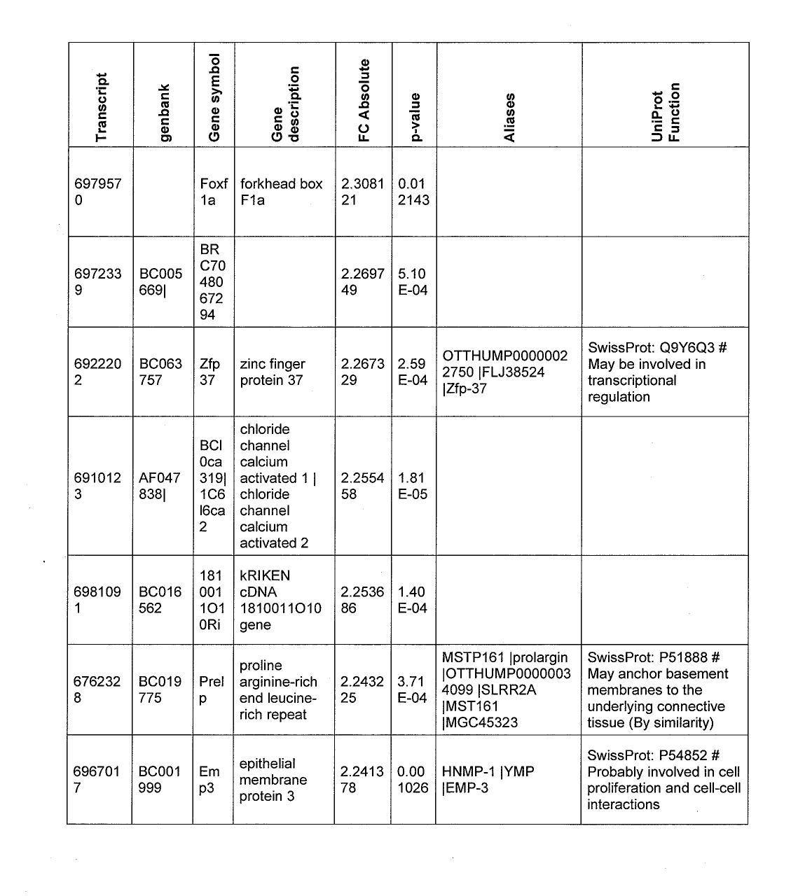

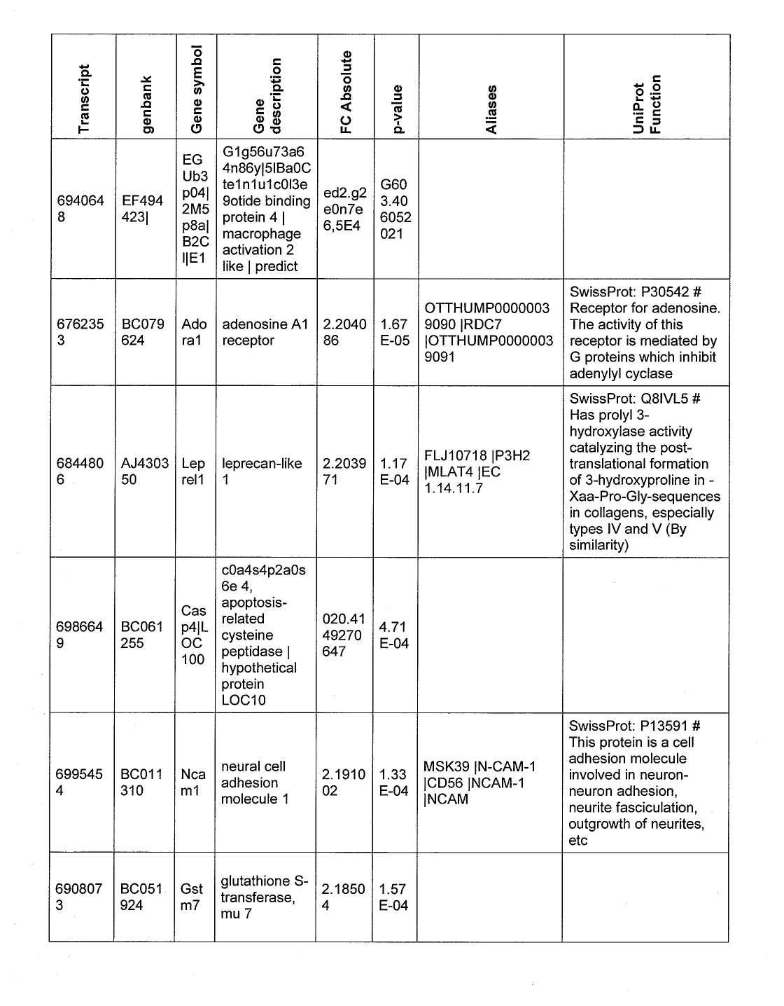

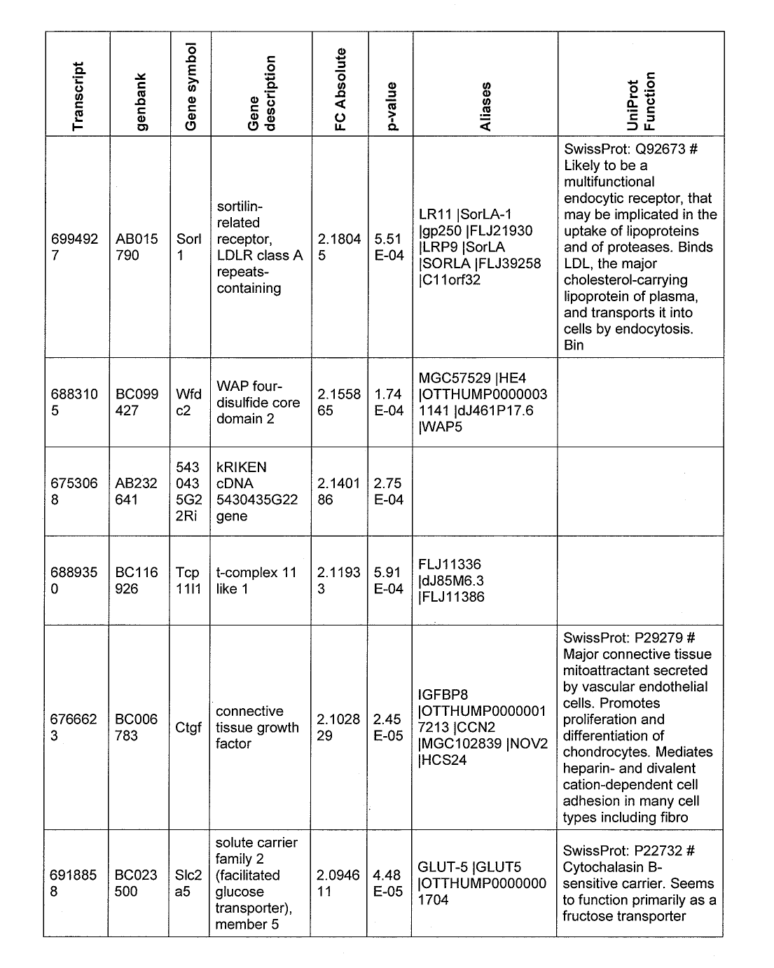

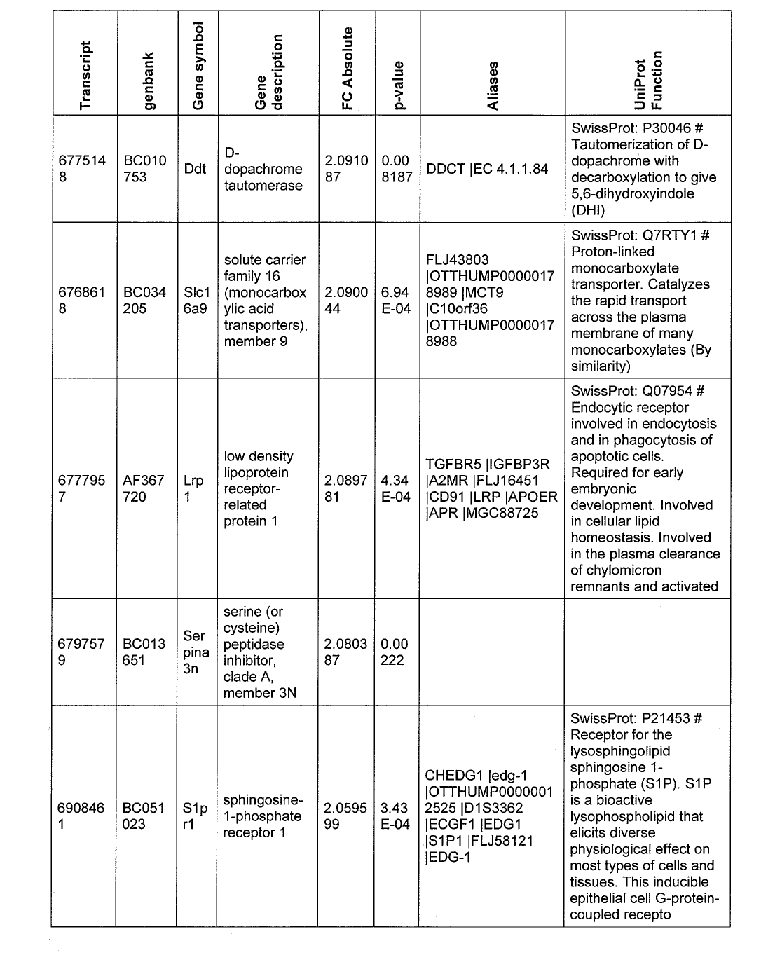

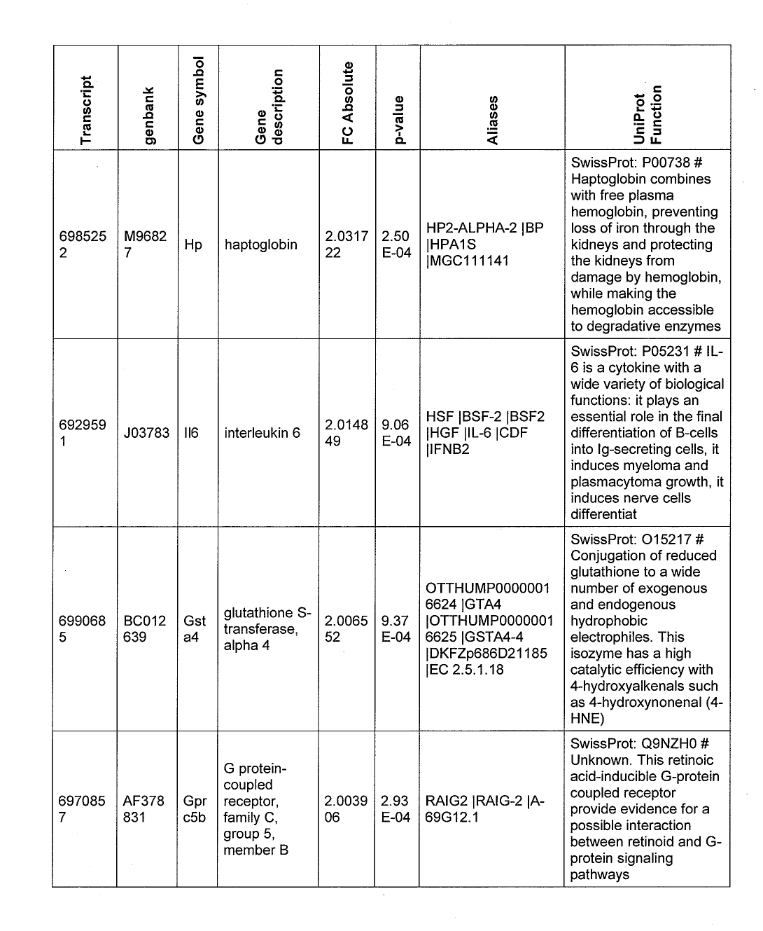

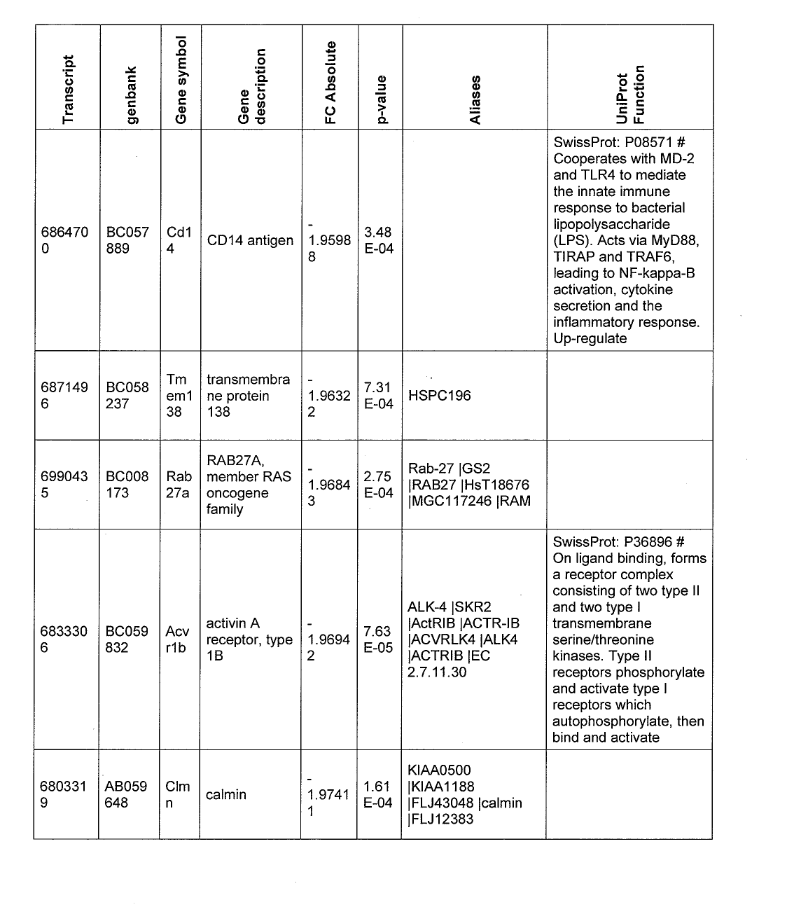

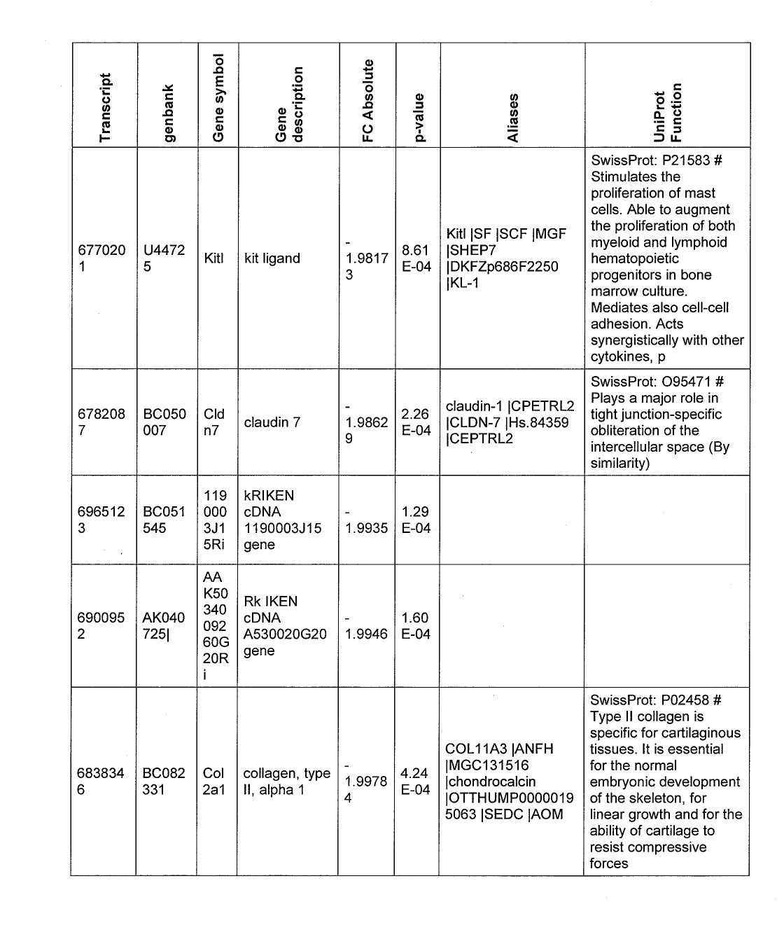

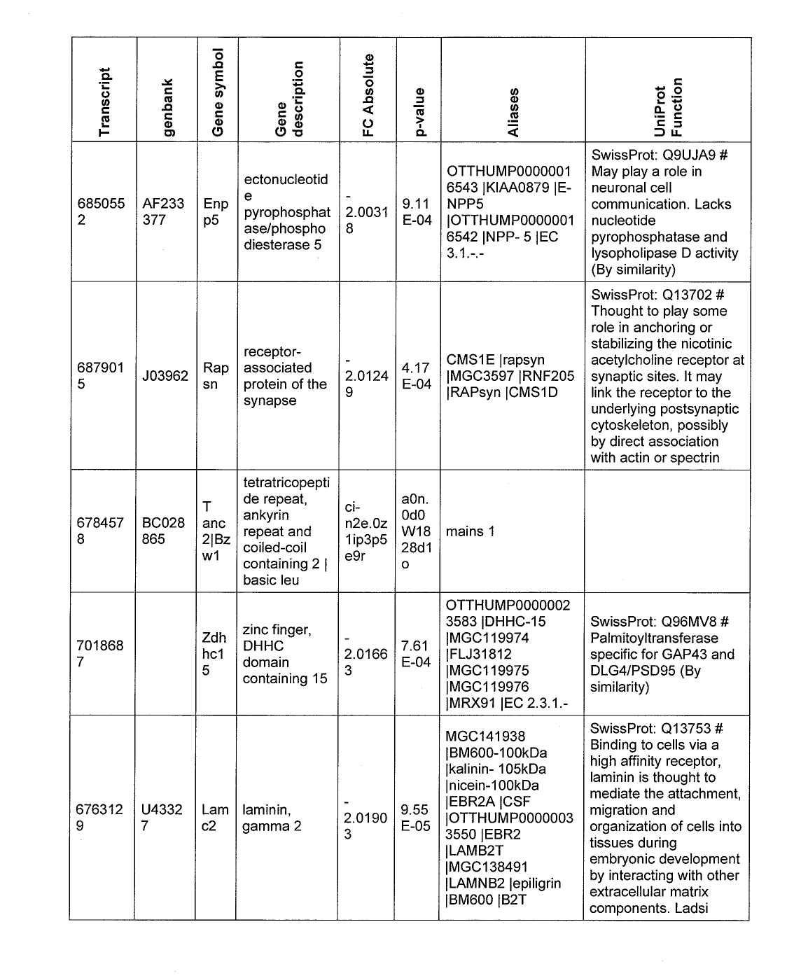

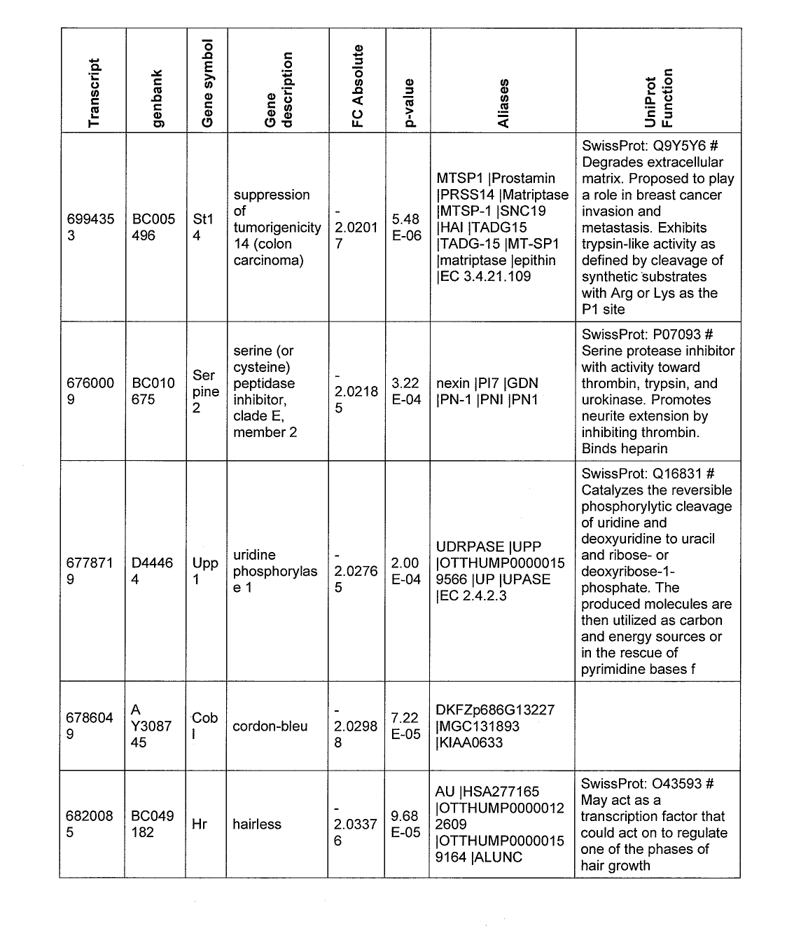

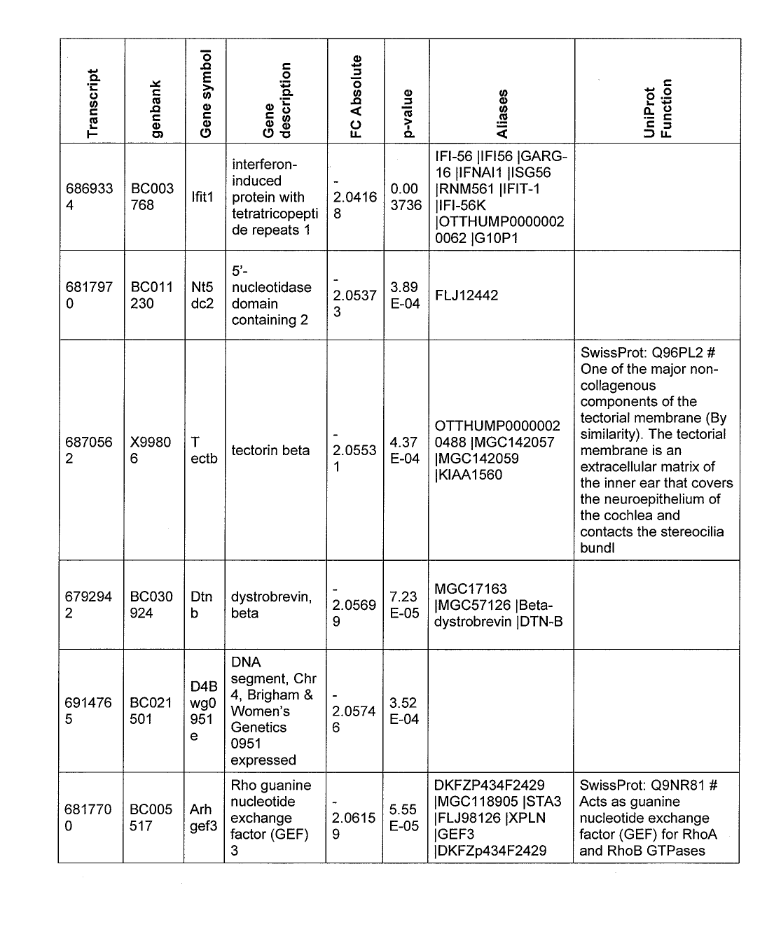

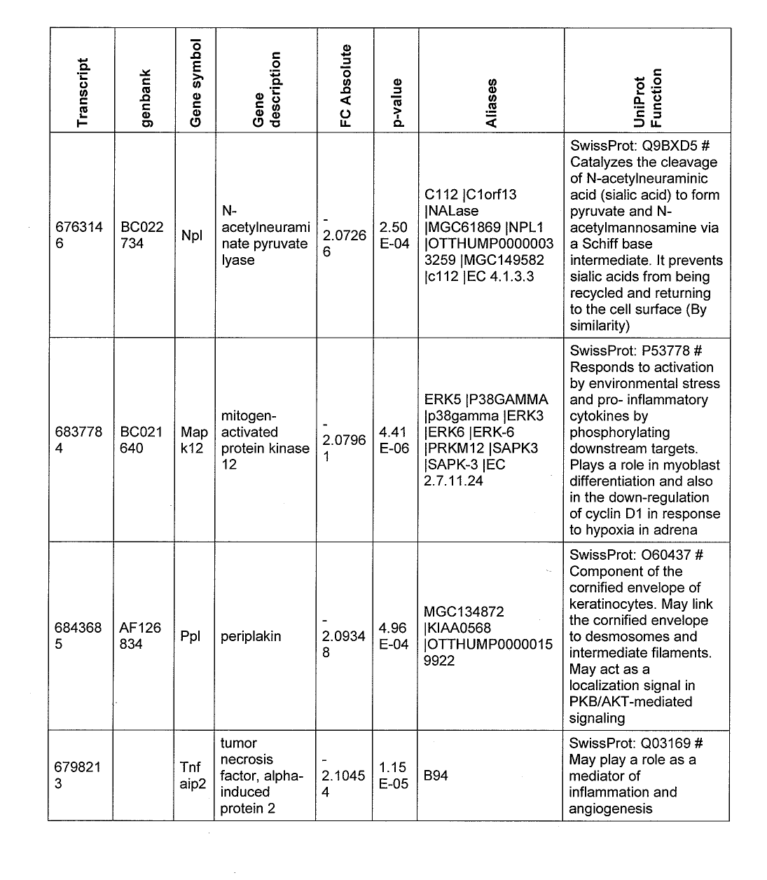

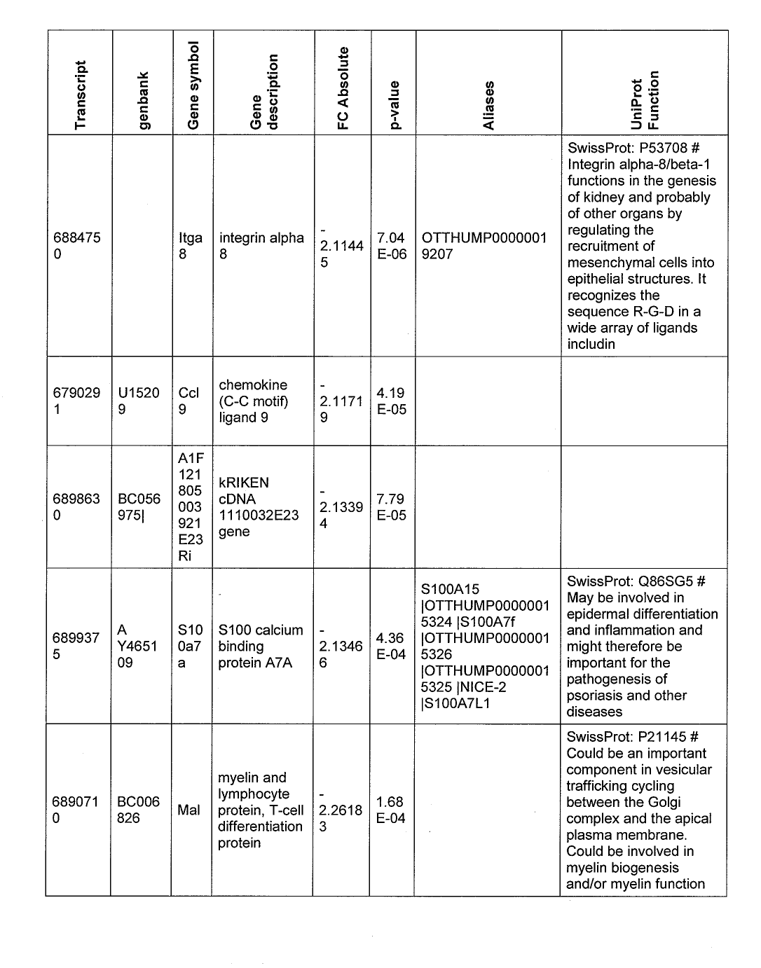

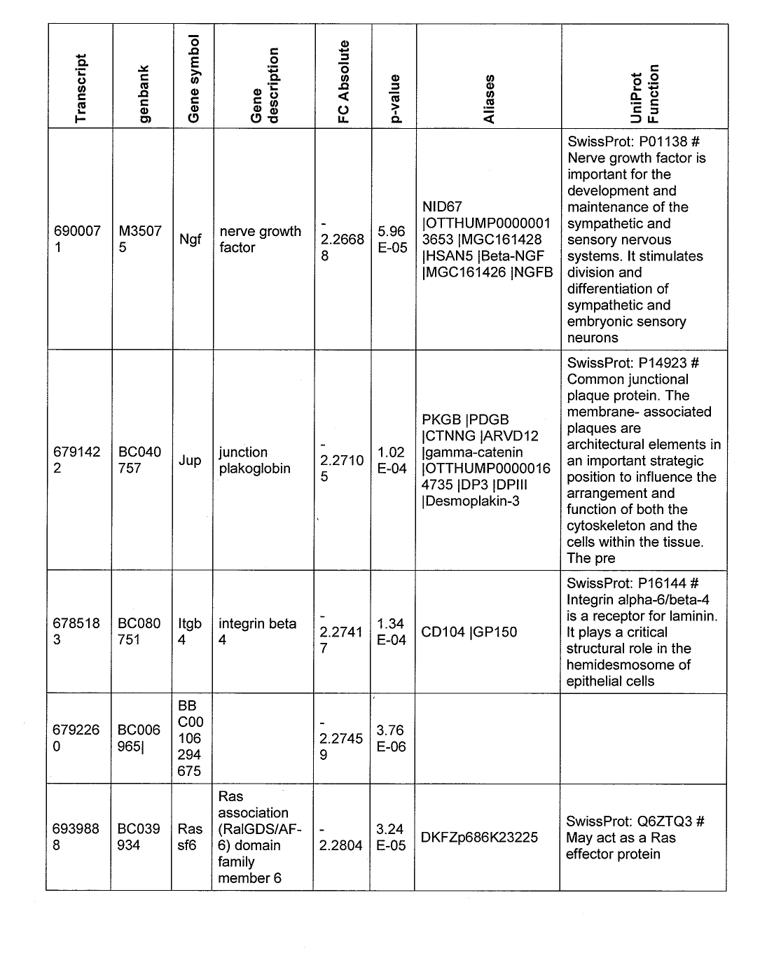

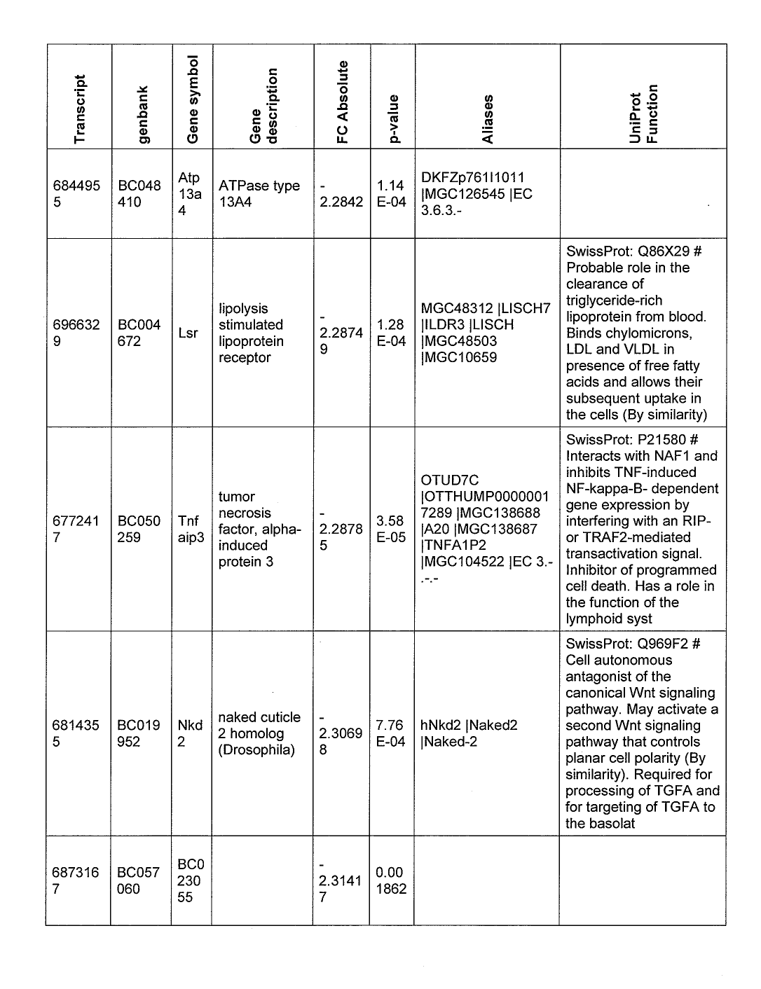

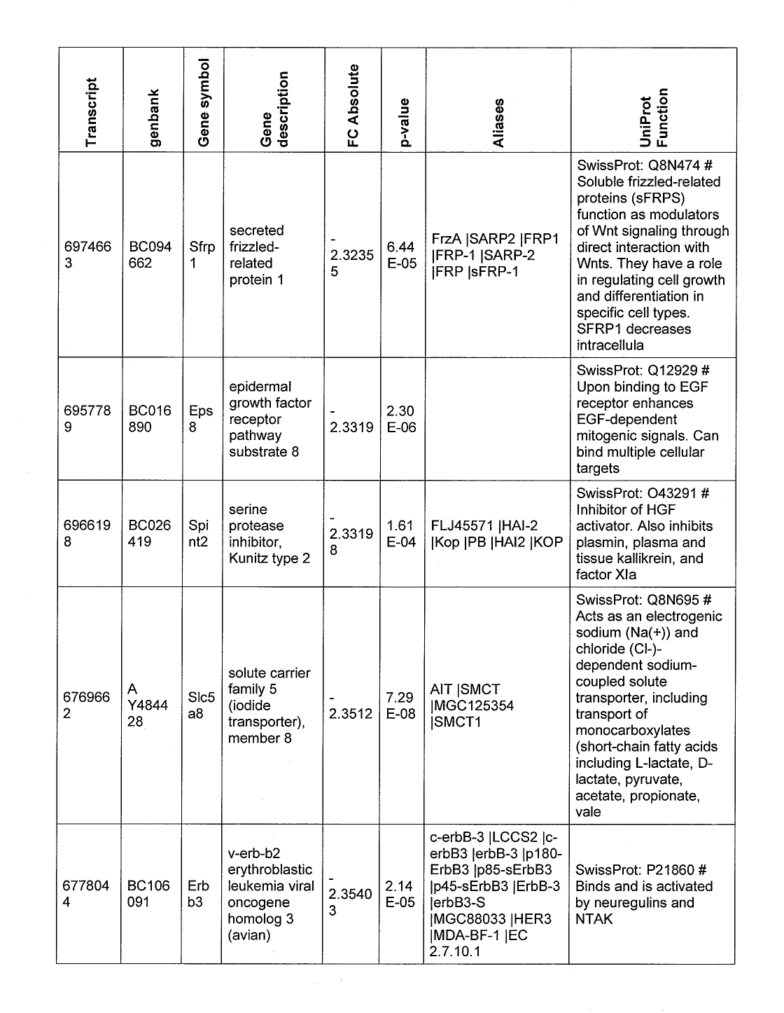

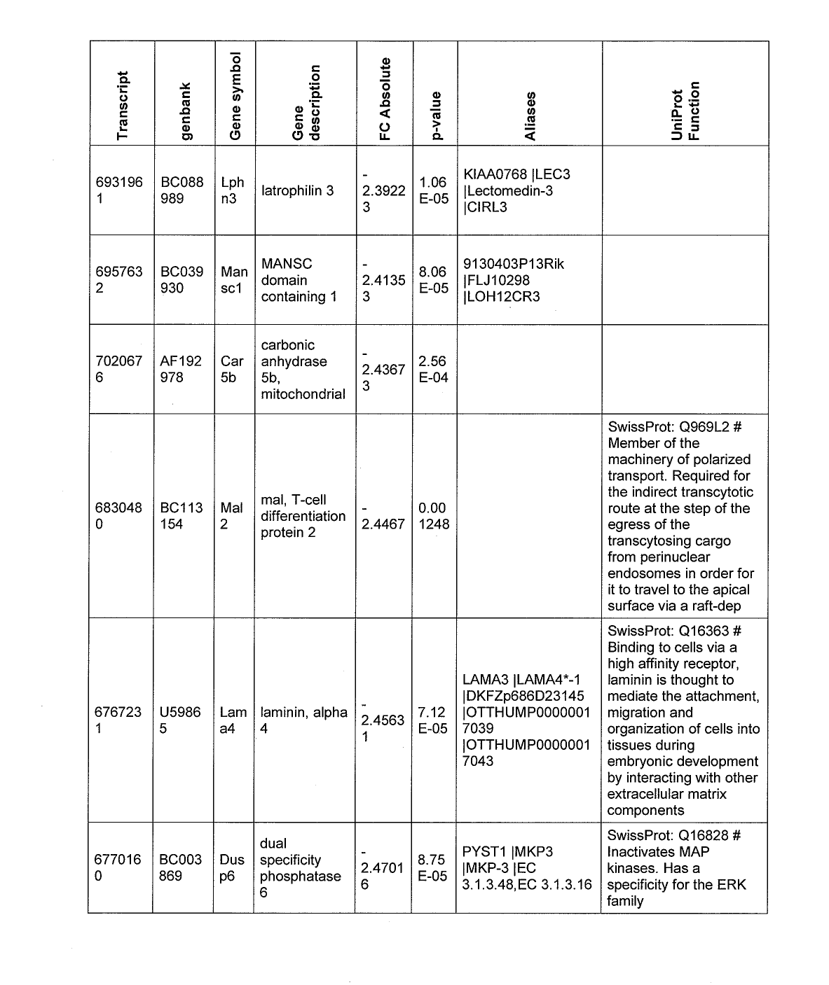

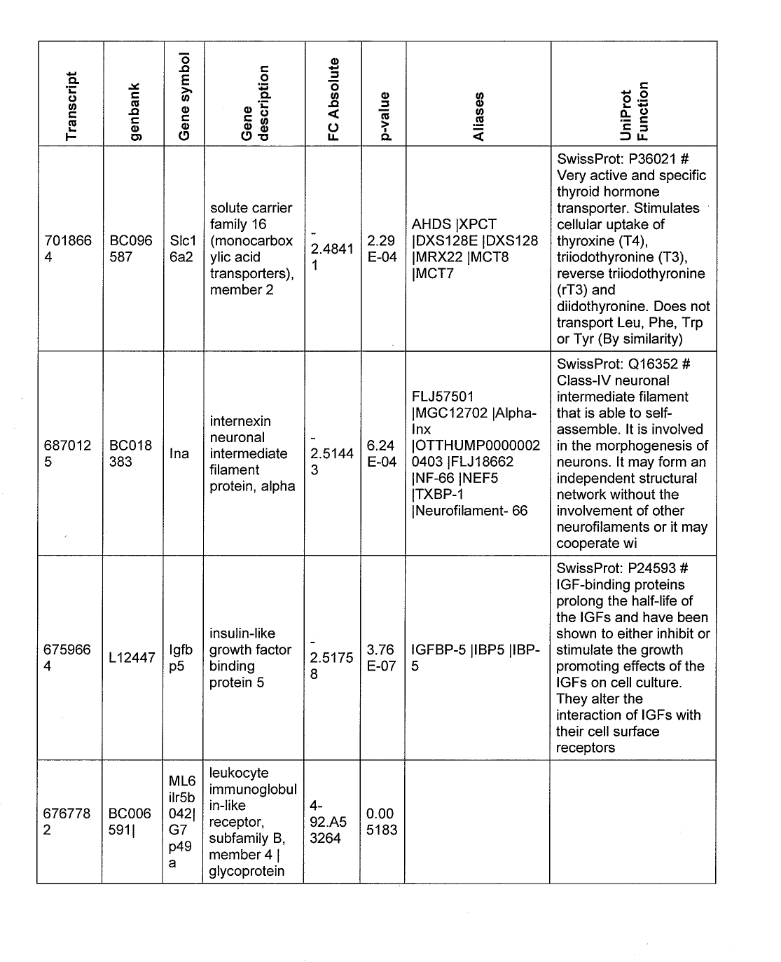

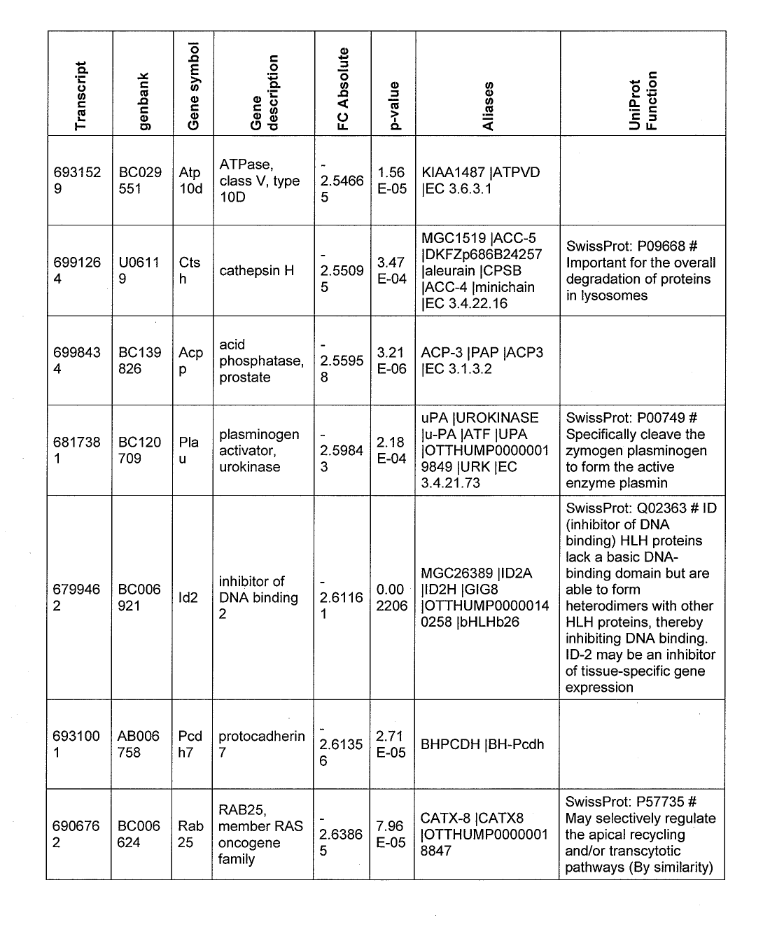

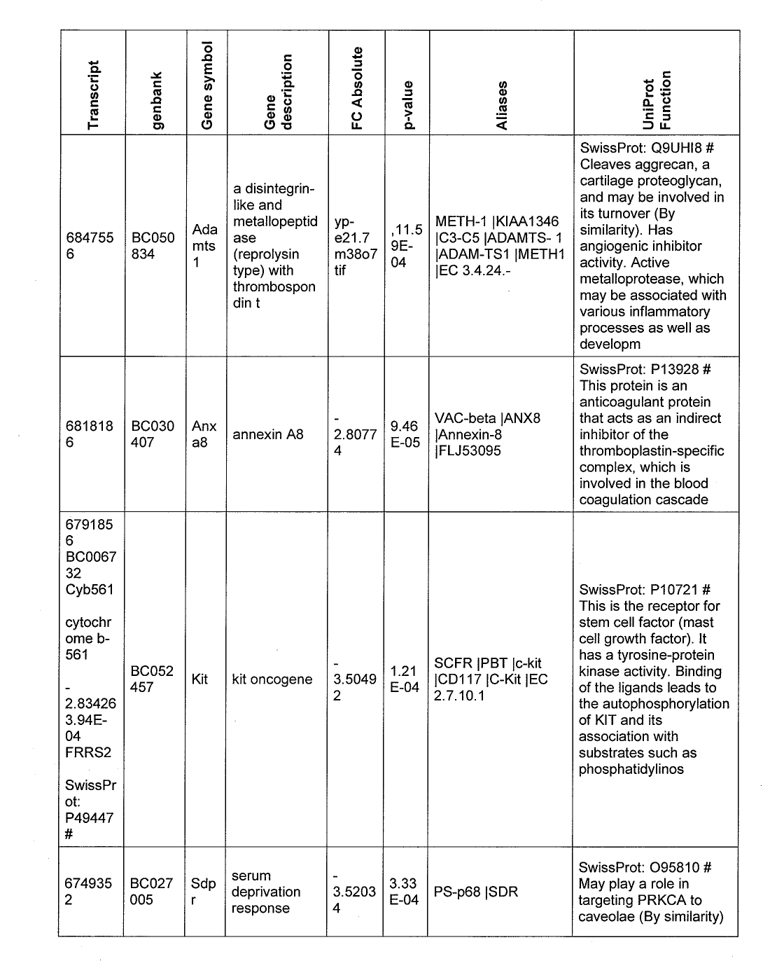

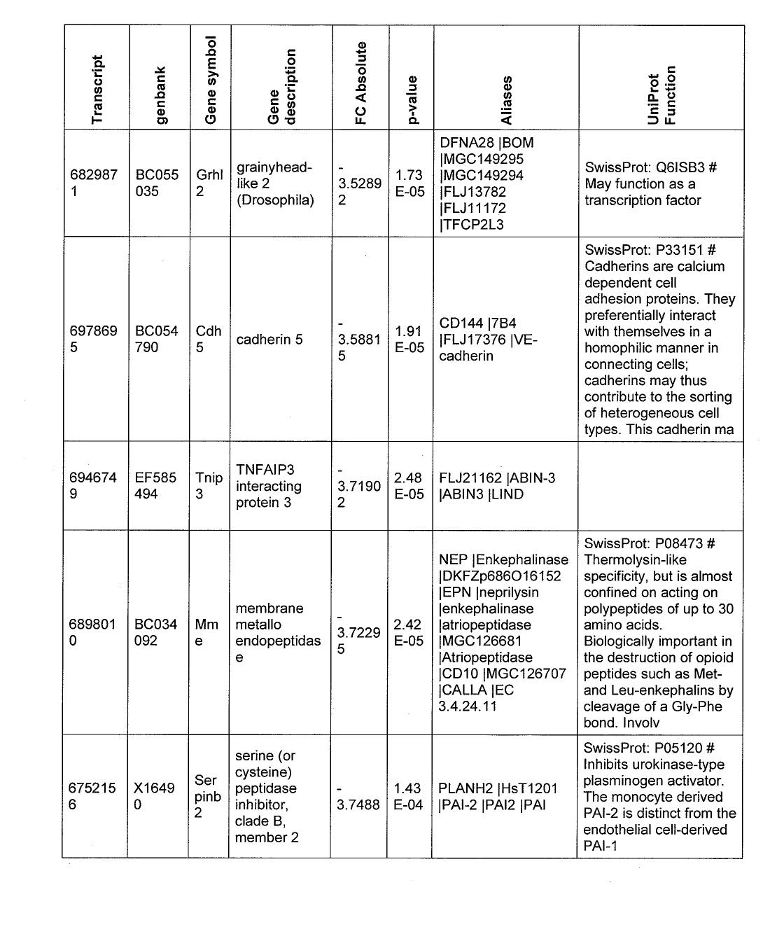

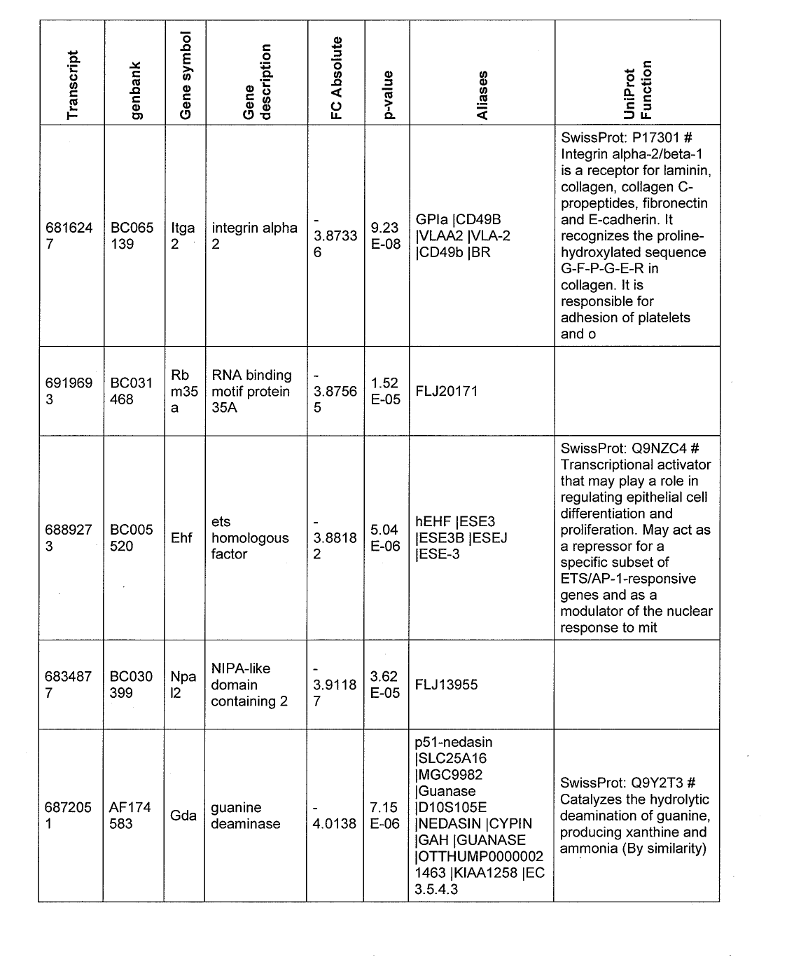

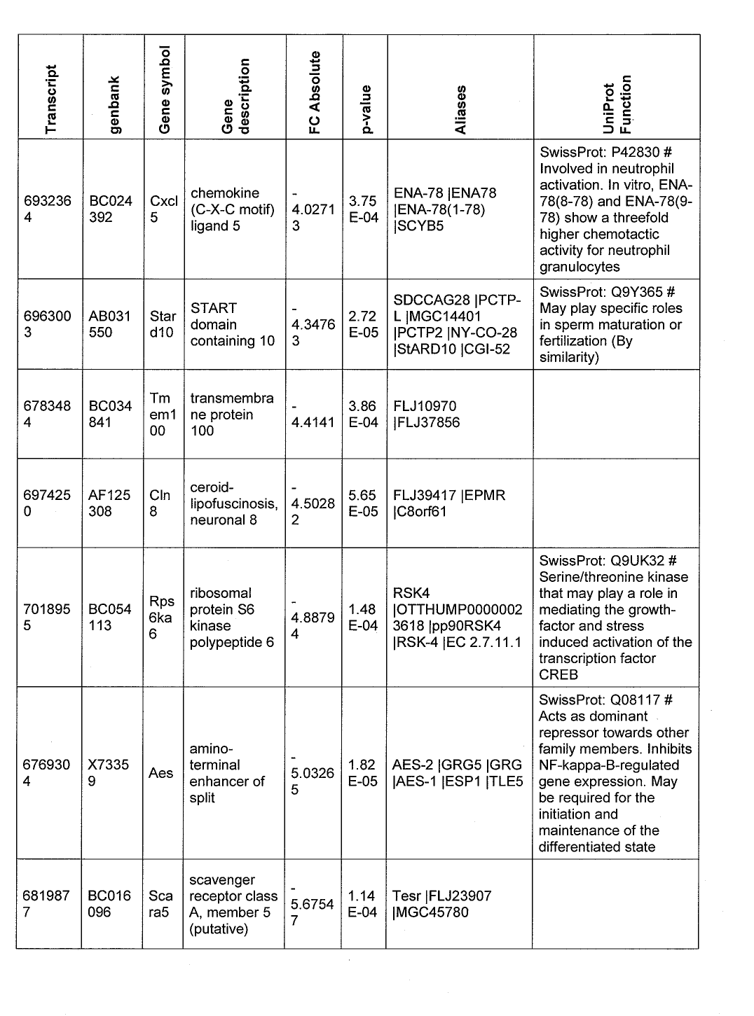

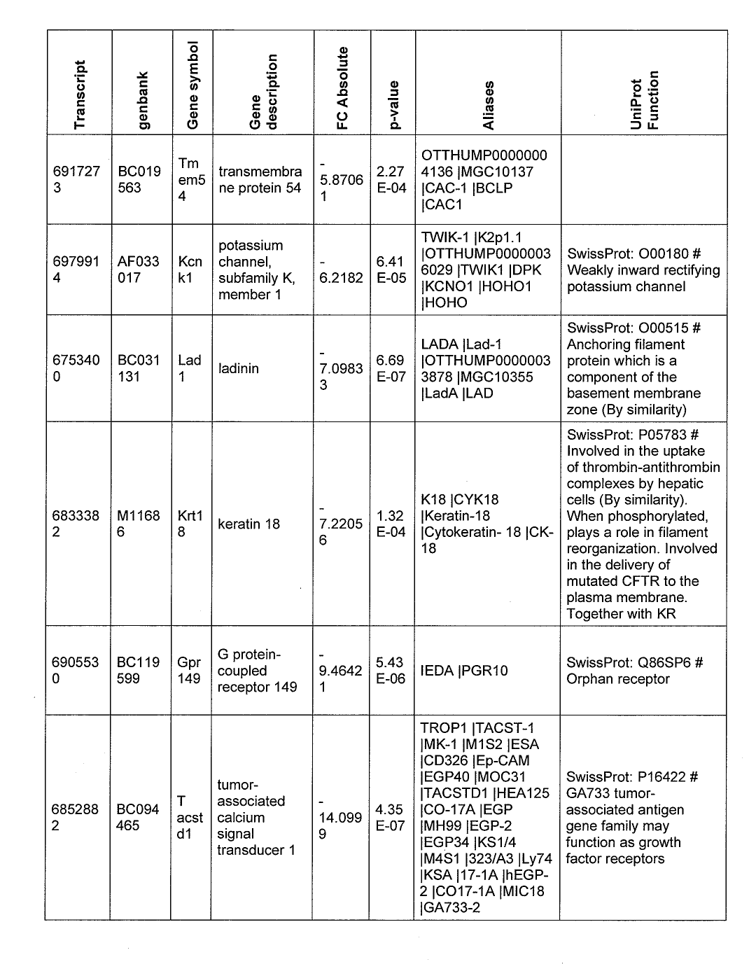

In order to examine further the mechanisms by which DACH1 inhibited cellular growth and angiogenesis, genome-wide expression studies were conducted of DACH1-transduced cells. Molecular pathway analysis was conducted with DAVID using Gene Ontology and KEGG pathway sets (see FIG. 14 and FIG. 10A). DACH1 repressed gene expression of signaling pathways governing hematopoietic cell lineage, cellular communication, blood vessel development, and multicellular organismal development. DACH1 induced an acute inflammation response and cytokine-cytokine receptor interaction (FIG. 10B). Several recent studies have suggested the molecular circuitry controlling stem cells may be active in certain tumors. Some of the key regulators of embryonic stem cell (ES) identity, Oct4, Sox2 and Nanog are expressed in specific tumors (Rodriguez-Pinilla S M, et al. Sox2: a possible driver of the basal-like phenotype in sporadic breast cancer. Mod Pathol 2007; 20: 474-81). An embryonic stem cell-like gene expression signature has been identified in poorly differentiated aggressive human tumors (Ben-Porath I, et al. An embryonic stem cell-like gene expression signature in poorly differentiated aggressive human tumors. Nat Genet 2008; 40: 499-507). Oct4, Sox2, Nanog are required for propagation of ES cells in culture. Comparison of DACH1 regulated genes in Met-1 cell to gene sets associated with ES cell identity via gene set enrichment analysis demonstrated DACH1 downregulates expression of Sox2, Oct4 gene targets, NOS targets (genes common to Nanog, Oct4 and Sox2) and a gene set overexpressed in hES cell lines (FIG. 10C).

Increased expression of the Sox2, Oct4, Nanog, and EKLF4 gene is associated with the stem cell phenotype. Whether expression of DACH1 could inhibit expression of genes associated with the cancer stem cell phenotype was examined. Quantitation of the relative expression of the ES cell markers (Sox2, Oct4, Nanog, KLF4 and c-Myc) was conducted using mRNA from the Met-1 cells expressing DACH1 or control (data not shown). QT-PCR analysis demonstrated a reduction in the abundance of Sox2, Nanog and KLF4. Each of these genes promotes stem cell expansion.

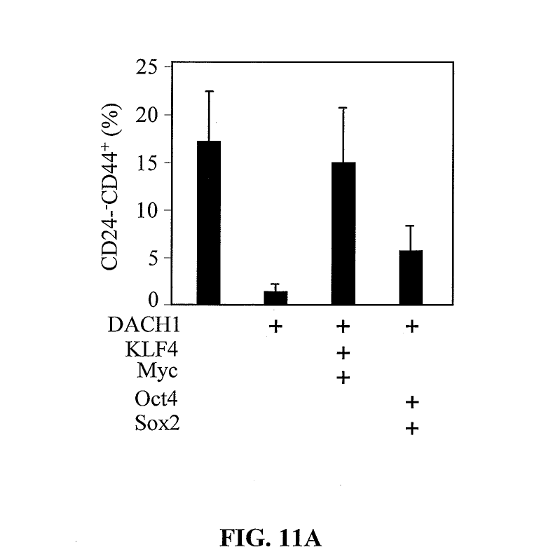

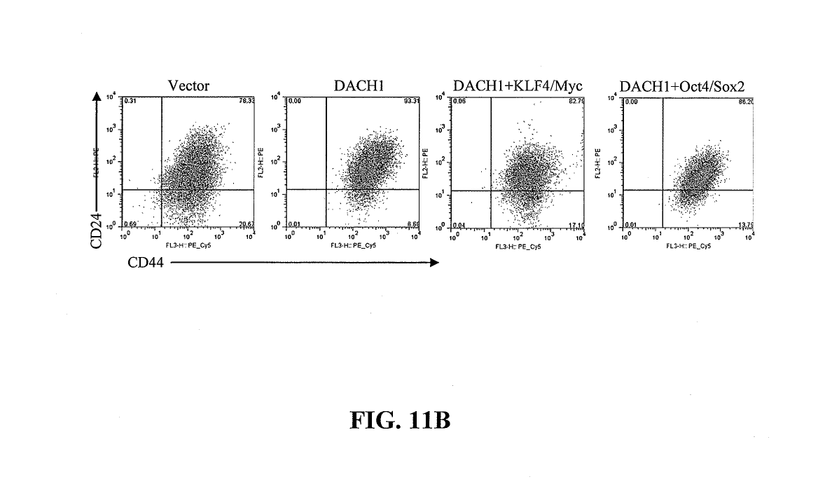

In order to determine the functional significance of KLF4 and Sox2 repression by DACH1, DACH1 transduced Met-1 cells were transfected with expression vectors encoding KLF4 or Sox2. A FACS analysis was conducted to examine the relative proportion of CD24⁻/CD44⁺ cells. DACH1 reduced the proportion of CD24⁻/CD44⁺ by ˜80%. Re-expression of KLF4/Myc or Sox2/Oct4 partially reversed the phenotype (FIG. 11A; FIG. 11B).

### DACH1 Binds Promoters of Genes Governing Progenitor Cell Expansion in ChIP and ChIP-Seq

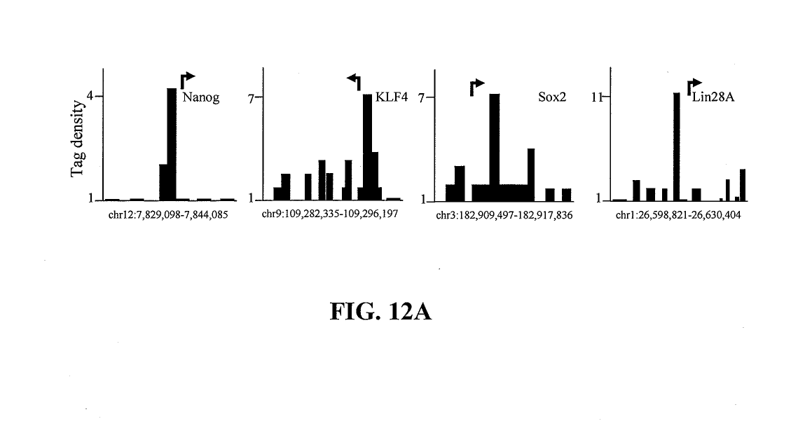

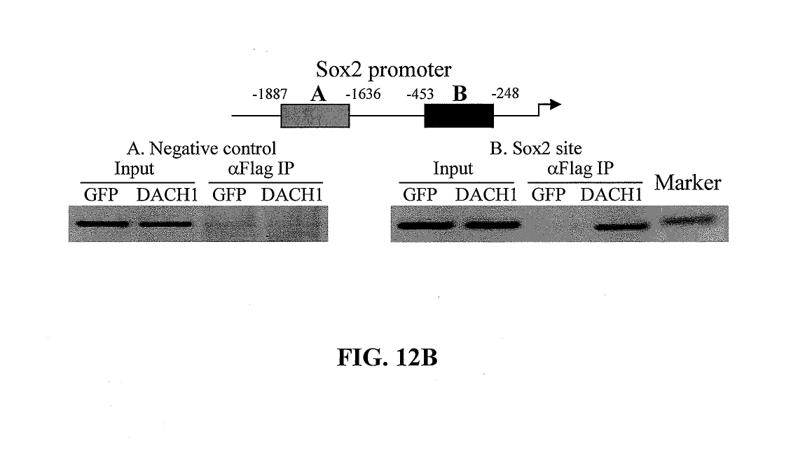

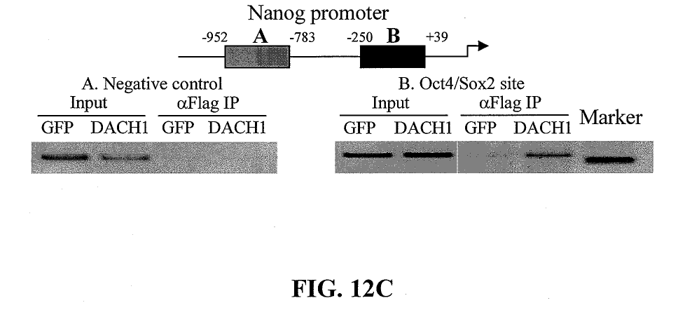

ChIP-Seq analysis was conducted of MDA-MB-231 cells expressing DACH1 in order to determine whether DACH1 bound the promoters of stem cell regulatory genes. DACH1 occupancy was identified at the Sox2, Nanog, KLF4 and Lin28 promoters (FIG. 12A). Sox2, KLF4 and Lin28 are known to play an important role in the maintenance of stem cell pluripotency. In order to examine further DACH1 physical association with the promoters of the Sox2 and Nanog genes in the context of local chromatin immunoprecipitation assays were conducted. Comparison was made using Met-1 cells expressing Flag tagged DACH1 or control vector. ChIP of the Sox2 promoter was conducted using oligonucleotides directed to either the distal or the proximal promoter. ChIP for DACH1 at the distal promoter failed to identify chromatin associated DACH1, however oligonucleotides directed to the proximal promoter including the Sox2 binding site demonstrated the recruitment of DACH1 (FIG. 12B). Similarly, the ChIP analysis of the Nanog promoter identified DACH1 recruitment to the proximal but not distal promoter region (FIG. 12C).

As DACH1 had reduced the expression of Sox2 and Nanog, studies were conducted to determine whether the Sox2 and Nanog genes were directly repressed by DACH 1. The promoter of the Sox2 and the Nanog genes were directly repressed by DACH1 expression (data not shown). Deletion of the DACH1 DS domain abrogated transcriptional repression DACH1 (data not shown).

### Methods of Treatment

The current studies provide several lines of evidence that DACH1 inhibits breast tumor stem cell expansion. DACH1 reduced the expression of the breast cancer stem cell markers (CD44^{high}/CD24^{low}) within the tumors. DACH1 also reduced the relative proportion of CD44^{high}/CD24^{low} Met-1 cells in tissue culture. DACH1 reduced the number and size of mammospheres. Only a small number of primary breast cancer cells known as breast tumor initiating cells (BTIC) give rise to secondary tumors and the growth of BTIC as non-adherent mammospheres serves as a useful surrogate of BTIC. shRNA to DACH1 reduced endogenous DACH1, and increased the number of mammospheres and the proportion of CD44⁺/CD24⁻ cells. DACH1 abundance was reduced in cell lines with features of breast cancer stem cells, and in the basal phenotype of human breast cancer. The expression profile of genes regulated by DACH, and the genes known to regulate embryonic stem (ES) cell features showed significant overlap. Expression of the key regulators of ES cell function Sox and Nanog were repressed by DACH1 in Met-1 cells and DACH repressed the Sox and Nanog gene promoters. The current studies are consistent with a model which DACH1 reduces the proportion of breast cancer stem cells.

DACH1 is known to regulate gene transcription indirectly through binding to DNA binding transcription factors (c-jun, Smad, SIX) (Wu K, et al. DACH 1 is a cell fate determination factor that inhibits Cyclin Dl and breast tumor growth. Mol Cell Biol 2006; 26: 7116-29; Li X, et al. Tissue-specific regulation of retinal and pituitary precursor cell proliferation. Science 2002; 297: 1180-3; Wu K, et al. DACH1 inhibits transforming growth factor-beta signaling through binding Smad4. J Biol Chem 2003; 278: 51673-84; and Wu K, et al. The Cell Fate Determination Factor DACH1 Inhibits c-Jun Induced Contact-Independent Growth. Mol Biol Cell 2007: 755-67). Herein, the transcriptional repression of the Sox and Nanog genes by DACH1 required a domain that is highly conserved from Drosophila to humans. The Dachshund box N (DS domain) shares significant amino acids with the Ski/Sno family. The DS domain is required for transcriptional repression of a subset of target genes and is required for HDAC1 recruitment by DACH1 in the context of local chromatin using ChIP assays. The DNA binding domain of DACH1 was required for repression of the BTIC phenotype assessed using Aldefluor staining. DACH 1 was recruited in the context of local chromatin to the proximal promoters of the Sox2 and Nanog promoters. Collectively these studies suggest DACH1 represses expression of Sox2, Nanog and KLF4. These findings are consistent with DACH1 expression reduced in the basal breast cancer phenotype and that the basal phenotype is known to overexpress Sox2 and that the basal phenotype displays features of breast cancer stem cells. Sox2 maintains stem cell properties and Sox2 downregulation correlates with loss of pluripotency and the induction of differentiation. Reexpression of KLF4/c-Myc or Sox2/Oct4 partially reversed the inhibition of the BTIC phenotype.

DACH1 reduced the proportion of CD44^{high}/CD24^{low} cells. Cancer stem cells can be enriched by sorting for CD44^{high}/CD24^{low} cells and characterized by their multipotentiality and their ability to self-renew (Al-Hajj M. Cancer stem cells and oncology therapeutics. Curr Opin Oncol 2007; 19: 61-4). The population of CD44^{high}/CD24^{low} was enriched in their capacity to produce mammospheres. The population of CD44^{high}/CD24^{low} cells was multipotential giving rise to both CD44^{high}/CD24^{low} and CD44^{high}/CD24^{high} populations after serial passage in tissue culture. These studies show DACH1 represses the proportion of breast cancer cells with multipotentiality characteristic of cancer stem cells.

Dac may have a role in progenitor cell function. In Drosophila, dac is expressed in progenitor of stem cells that give rise to several distinct organ cellular populations including muscle, neurons and gonadal germ cells. Dac is expressed in neural progenitors (neuroblasts) of the mushroom body, a brain structure present in most arthropods. These neuroblasts divide in a stem cell mode and produce lineages of 10-20 neurons. Dac is thought to play a role in specifying the structural fate of Kenyon cell axons and mushroom body neuropile are drastically abolished in the pupa of dac null mutants (Noveen A, et al. Early development of the Drosophila mushroom body: the roles of eyeless and dachshund. Development 2000; 127: 3475-88). In the mammalian cells, DACH1 is expressed in the developing eye, ear, limb and mammary epithelium. Although Dach1 gene deletion in the mouse is perinatal lethal, expression studies in the murine embryo suggest an important role for Dach1 in cell-fate determination. Thus Dac is expressed in embryonic progenitor cells, and expression is lost upon terminal differentiation. DACH1 expression is reduced in tumors (breast, prostate, uterus) (Wu K, et al. DACH 1 is a cell fate determination factor that inhibits Cyclin D1 and breast tumor growth. Mol Cell Biol 2006; 26: 7116-29; Wu K, et al. The cell fate determination factor dachshund inhibits androgen receptor signaling and prostate cancer cellular growth. Cancer Res 2009; 69: 3347-55) correlating with poor prognosis. DACH1 re-expression in breast cancer cells reduced the proportion of cells with features of cancer stem cells. In the studies described herein, DACH1 reduced cellular invasiveness and reduced the proportion of CD44^{high}/CD24^{low} cells. Analysis of the mechanism by which DACH1 regulates the proportion of BTIC, demonstrated the role for a secreted factor. The conditioned medium from DACH1 transduced Met-1 cells recapitulated the effect of DACH1 transduction of Met-1 cells to reduce the proportion of CD44⁺/CD24^{-low} cells.

### Example

### Methylation of DACH1 Promoter

DACH1 expression is regulated by epigenetic modification. DNA methylation is a common mechanism leading to tumor suppressor inactivation. By using a combination methylation enzyme digestion and PCR amplification, it has been shown that DACH1 promoter is methylated in breast cancer cell lines (data not shown). Treatment of different breast cancer cell lines with 5-aza-2′-deoxycytidine induced DACH1 mRNA expression (data not shown).

All references cited herein are incorporated herein by reference in their entirety. To the extent publications and patents or patent applications incorporated by reference contradict the disclosure contained in the specification, the specification is intended to supersede and/or take precedence over any such contradictory material.

To the extent publications and patents or patent applications incorporated by reference herein contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein.

Terms and phrases used in this application, and variations thereof, unless otherwise expressly stated, should be construed as open ended as opposed to limiting. As examples of the foregoing, the term ‘including’ should be read to mean ‘including, without limitation’ or the like; the term ‘comprising’ as used herein is synonymous with ‘including,’ ‘containing,’ or ‘characterized by,’ and is inclusive or open-ended and does not exclude additional, unrecited elements or method steps; the term ‘example’ is used to provide exemplary instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ desired,' or ‘desirable,’ and words of similar meaning should not be understood as implying that certain features are critical, essential, or even important to the structure or function of the invention, but instead as merely intended to highlight alternative or additional features that may or may not be utilized in a particular embodiment of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise. In addition, as used in this application, the articles ‘a’ and ‘an’ should be construed as referring to one or more than one (i.e., to at least one) of the grammatical objects of the article. By way of example, ‘an element’ means one element or more than one element.

The presence in some instances of broadening words and phrases such as ‘one or more’, ‘at least’, ‘but not limited to’, or other like phrases shall not be read to mean that the narrower case is intended or required in instances where such broadening phrases may be absent.

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific embodiments and examples described herein, but rather to also cover all modification and alternatives coming with the true scope and spirit of the invention.

1-76. (canceled)

77. A method for treating a subject with cancer, comprising:
determining an expression level of a nucleic acid encoding DACH1 or DACH1 protein in a sample obtained from a subject in need of treatment for cancer;
selecting a treatment for the subject based on the determined expression level; and
administering the selected treatment to the subject.

78. The method of claim 77, further comprising comparing the expression level of the nucleic acid encoding DACH1 or DACH1 protein in the sample obtained from the subject to that of at least one of target cells with markers of cancer stem cells or one or more nucleic acids encoding a gene selected from the group consisting of Sox2 and Klf4.

79. The method of claim 78, wherein the markers of cancer stem cells are stem cell surface markers.

80. The method of claim 77, further comprising:
measuring an expression level of one or more nucleic acids encoding a gene selected from the group consisting of Sox2, Klf4, and Nanog or one or more proteins selected from the group consisting of SOX2, KLF4, and NANOG in a sample obtained from the subject; and
comparing the expression level of the nucleic acid encoding DACH1 or DACH1 protein and the expression level of the one or more nucleic acids encoding a gene selected from the group consisting of Sox2, Klf4, and Nanog or the one or more proteins selected from the group consisting of SOX2, KLF4, and NANOG in the sample to an expression level of a nucleic acid encoding DACH1 or DACH1 protein and an expression level of the one or more nucleic acids encoding a gene selected from the group consisting of Sox2, Klf4, and Nanog or the one or more proteins selected from the group consisting of SOX2, KLF4, and NANOG in normal tissue or cancerous tissue with a known metastatic potential.

81. The method of claim 80, wherein said comparing expression levels comprises a decreased expression level of a nucleic acid encoding DACH1 or DACH1 protein and an increased expression level of the nucleic acid encoding a gene selected from the group consisting of Sox2, Klf4, and Nanog or one or more proteins selected from the group consisting of SOX2, KLF4, and NANOG, and said selecting comprises selecting a treatment to increase the expression level of DACH1 in a tumor cell of the subject.

82. The method of claim 81, wherein the decrease in expression level is statistically significant, and wherein the increase in expression level is statistically significant.

83. The method of claim 77, wherein administering the selected treatment to the subject comprises administering a DNA-methyltransferase inhibitor to the subject.

84. The method of claim 83, wherein the DNA-methyltransferase inhibitor comprises a compound selected from the group consisting of 5-azacytidine and 5-aza-2′-deoxycytidine.

85. The method of claim 77, wherein administering the selected treatment to the subject comprises administering to the subject an anti-IL8 therapy.

86. The method of claim 85, wherein the anti-IL8 therapy comprises an immunoneutralizing antibody.

87. The method of claim 77, wherein the sample comprises the phenotype estrogen receptor negative, progesterone receptor negative and ERB2 negative.

88. The method of claim 77, wherein determining the expression level a nucleic acid encoding DACH1 comprises measuring the level of DACH1 mRNA in a cell of said sample.

89. The method of claim 77, wherein determining an expression level of DACH1 protein in said sample comprises an immunoblot analysis.

90. The method of claim 77, wherein administering the selected treatment to the subject comprises administering an isolated nucleic acid encoding DACH1 or a DACH1 protein to a subject in need thereof.

91. The method of claim 77, wherein administering the selected treatment to the subject comprises administering a tissue specific promoter, whereby transcription in specific cells is effected so as to reduce toxicity in non-targeted tissues, wherein the tissue specific promoter is selected from the group consisting of adipose differentiation related protein promoter, whey acidic protein promoter, β casein promoter, lactalbumin promoter, β-lactoglobulin promoter, prostate specific antigen, probasin, prostatic acid phosphatase, prostate-specific glandular kallikrein, CFTR, human cytokeratin IS (K 18), pulmonary surfactant protein A, pulmonary surfactant protein B, pulmonary surfactant protein C, pulmonary surfactant protein CC-10, and pulmonary surfactant protein Pi.

92. The method of claim 77, wherein the cancer is selected from breast cancer, kidney cancer, lung cancer, brain cancer, endometrial cancer, ovarian cancer, pancreatic cancer, and prostate cancer.

93. The method of claim 92, wherein the cancer comprises the phenotype estrogen receptor negative, progesterone receptor negative and ERB2 negative.

94. The method of claim 92, whereby a reduction in an expression level of one or more genes selected from the group consisting of Sox2, Nanog, and Klf4 is obtained.

95. The method of claim 92, whereby a reduction is obtained in an expression level of one or more genes associated with a signaling pathway selected from the group consisting of hematopoietic cell lineage, cellular communication, blood vessel development, and multicellular organismal development.

96. The method of claim 92, whereby a decrease is obtained in an expression level of one or more genes associated with an acute inflammation response and cytokine-cytokine receptor interactions.

97. The method of claim 92, wherein the cancer comprises a solid tumor.

98. The method of claim 92, wherein a reduction in a proportion of a cancer stem cell marker in the solid tumor is obtained.

99. The method of claim 92, wherein a reduction in a proportion of CD24^{-low} cells in the solid tumor is obtained.

100. The method of claim 92, wherein a decreased expression level of a nucleic acid encoding DACH1 or DACH1 protein is indicative of the metastatic potential of the tumor.


SEQUENCE LISTING (18 sequences)

SEQ ID NO: 1 - DNA, 26 bp, Artificial Sequence
  Nanog forward olignucleotide
cagaaaaaccagtggttgaagactag

SEQ ID NO: 2 - DNA, 21 bp, Artificial Sequence
  Nanog reverse olignucleotide
gcaatggatgctgggatactc

SEQ ID NO: 3 - DNA, 24 bp, Artificial Sequence
  Oct4 forward olignucleotide
ctgtagggagggcttcgggcactt

SEQ ID NO: 4 - DNA, 24 bp, Artificial Sequence
  Oct4 reverse olignucleotide
ctgagggccaggcaggagcacgag

SEQ ID NO: 5 - DNA, 25 bp, Artificial Sequence
  Sox2 forward olignucleotide
ggcagctacagcatgatgcaggagc

SEQ ID NO: 6 - DNA, 24 bp, Artificial Sequence
  Sox2 reverse olignucleotide
ctggtcatggagttgtactgcagg

SEQ ID NO: 7 - DNA, 21 bp, Artificial Sequence
  KLF4 forward olignucleotide
tgccagaccagatgcagtcac

SEQ ID NO: 8 - DNA, 21 bp, Artificial Sequence
  KLF4 reverse olignucleotide
gtagtgcctggtcagttcatc

SEQ ID NO: 9 - DNA, 19 bp, Artificial Sequence
  c-Myc forward olignucleotide
tgagcccctagtgctgcat

SEQ ID NO: 10 - DNA, 19 bp, Artificial Sequence
  c-Myc reverse olignucleotide
agcccgactccgacctctt

SEQ ID NO: 11 - DNA, 20 bp, Artificial Sequence
  Forward oligonucleotide
gcagtgagaggggtggacta

SEQ ID NO: 12 - DNA, 20 bp, Artificial Sequence
  Reverse olignucleotide
ctcccctcatctaccccaac

SEQ ID NO: 13 - DNA, 22 bp, Artificial Sequence
  Forward oligonucleotide
cgcagaaacaatggcacaccac

SEQ ID NO: 14 - DNA, 22 bp, Artificial Sequence
  Reverse oligonucleotide
ccgttttcagcaacaggtcacg

SEQ ID NO: 15 - DNA, 30 bp, Artificial Sequence
  Forward oligonucleoide
ggcaaactttgaacttgggatgtggaaata

SEQ ID NO: 16 - DNA, 30 bp, Artificial Sequence
  Reverse oligonucleotide
ctcagccgtctaagcaatggaagaagaaat

SEQ ID NO: 17 - DNA, 26 bp, Artificial Sequence
  Forward oligonucleoide
ggatgtctttagatcagaggatgccc

SEQ ID NO: 18 - DNA, 25 bp, Artificial Sequence
  Reverse oligonucleotide
ccacagaaagagcaagacaccaacc